(19) United States
(12) Patent Application Publication
(10) Pub. No.: US 2025/0073268 A1
(43) Pub. Date: Mar. 6, 2025

(54) CHIMERIC CD40 POLYPEPTIDES AND METHODS OF USE IN IMMUNOTHERAPY

(71) Applicant: Providence Health & Services - Oregon, Portland, OR (US)

(72) Inventors: Eric Tran, Portland, OR (US); Myungkyu Jang, Portland, OR (US); Timothy Erickson, Tampa, FL (US)

(73) Assignee: Providence Health & Services - Oregon, Portland, OR (US)

(21) Appl. No.: 18/726,716

(22) PCT Filed: Jan. 4, 2023
(86) PCT No.: PCT/US2023/060079

§ 371 Date: Jul. 3, 2024

Related U.S. Application Data
(60) Provisional application No. 63/296,623, filed on Jan. 5, 2022.

(51) Int. Cl. A61K 35/17; A61K 39/00; C07K 14/705; C07K 14/715; C07K 14/72; C12N 5/0783

(52) U.S. Cl. CPC A61K 35/17; A61K 40/11; A61K 40/30; C07K 14/70521; C07K 14/70578; C07K 14/70596; C07K 14/7153; C07K 14/7155; C07K 14/72; C12N 5/0636; C07K 2319/03; C12N 2510/00

(57) ABSTRACT

Modified T cells comprising ectopic CD40 proteins (such as chimeric CD40 proteins including a CD40 extracellular domain and a heterologous intracellular domain) are provided. Also provided are compositions, including pharmaceutical formulations, comprising the modified T cells, and methods for increasing T cell-mediated tumor cell-specific cytotoxicity using the same. Methods of treating a subject with cancer including administering to the subject the modified T cells, thereby activating an innate immune response and/or an adaptive immune response in the subject are also provided.

### CROSS REFERENCE TO RELATED APPLICATIONS

This application claims the benefit of U.S. Provisional Application No. 63/296,623, filed Jan. 5, 2022, which is incorporated by reference in its entirety.

### FIELD

This disclosure generally relates to compositions and methods of immunotherapy, particularly compositions and methods utilizing modified T-cells expressing CD40 proteins, such as chimeric CD40 polypeptides.

### BACKGROUND

CD40 is a member of the tumor necrosis factor (TNF) receptor superfamily and is expressed by antigen-presenting cells (APCs) such as B cells, dendritic cells, monocytes, and macrophages. CD40 is not normally expressed by T cells, but can be transiently upregulated on T cells upon activation and, when engaged by its ligand CD40L, can promote T-cell responses.

Although immunotherapy can effectively treat some patients with cancer, most patients still do not respond to therapy. Agonistic antibodies targeting CD40 are being tested as an immunotherapy for cancer patients based on their ability to activate APCs and promote antitumor immunity. Agonistic CD40 therapy that activates APCs appears to have biological activity in patients with cancer, but clinical responses are low.

### SUMMARY

Disclosed herein are modified T cells that include or express an ectopic CD40 protein. In some embodiments, the modified T cell includes a polynucleotide including a nucleotide sequence encoding an ectopic CD40 protein operably linked to a heterologous promoter. In some examples, the ectopic protein includes a polypeptide with at least 95% sequence identity to the amino acid sequence of SEQ ID NO: 1.

In other examples, modified T cells that include or express a chimeric CD40 protein including a CD40 extracellular domain and a heterologous intracellular domain, such as modified T cells including a nucleic acid encoding the chimeric CD40 polypeptide operably linked to a promoter. In some examples, the heterologous intracellular domain (or portion thereof) is from a member of the CD28 family (such as CD28 or ICOS), CD2, CD4, CD8, a cytokine receptor (such as CSF2RB, GMCSFRa, IL3Ra, IL2Rb, IL2Rg (common gamma chain), IL9R, IL7Ra, or TSLPR), a member of the TNFR family (such as 4-1BB, OX40, CD27, CD30, GITR, or TNFR2), a TNF ligand (such as RANKL, LIGHT, FasL, 41BBL, CD30L, CD27L, OX40L, TWEAK, BAFF, TRAIL, EDA, or GITRL), a member of the TLR family (such as TLR1, TLR2, TLR3, TLR4, TLR5, TLR6, TLR7, TLR8, TLR9, or TLR10), a member of the receptor tyrosine kinases (RTK) family (such as IGF1R, EGFR, TRKA, TRKB, TRKC, MUSK, MET, AXL, TIE2, EPHA (EPHA1, EPHA3, EPHA7, or EPHA8), EPHB (EPHB1, EPHB2, EPHB3, or EPHB6), DDR1, ROS1, LTK, or ALK), a member of the integrins family (such as CD11A or CD18 (beta-2 integrin)), or a member of the human killer immunoglobulin-like receptor (KIR) family (such as NKG2D, CD94, NKP46, NKP30, NKP44, NKP80, KIR2DS1, CLEC2D, or KIR3DS1). In some embodiments, the transmembrane domain is a CD40 transmembrane domain. In other embodiments, the transmembrane domain is a heterologous transmembrane domain, for example, a transmembrane domain from the same protein as the heterologous intracellular domain. In some examples, the CD40 extracellular domain has at least 95%, at least 97%, at least 98%, or at least 99% sequence identity to the amino acid sequence of SEQ ID NO: 2.

In embodiments, the CD40 protein is a human CD40 protein. In additional embodiments, the heterologous promoter operably linked to the nucleotide sequence encoding the ectopic CD40 protein or chimeric CD40 polypeptide is a constitutive promoter or an inducible promoter.

In some embodiments, the nucleotide sequence encoding the ectopic CD40 protein or chimeric CD40 polypeptide operably linked to a heterologous promoter is included in a vector, such as an expression vector. In some examples, the vector is a viral vector, such as a lentivirus vector or a gamma-retrovirus vector.

In additional embodiments, the modified T cell is a human T cell. In some examples, the modified T cell is produced from a T cell isolated from a blood sample. In some examples, the modified T cell is a tumor-reactive T cell. The tumor-reactive T cell may be isolated from a tumor sample (e.g., a tumor-infiltrating lymphocyte) or from a blood sample.

In some embodiments, the modified T cell may also include a heterologous T cell receptor (TCR), a chimeric antigen receptor (CAR), or both. In particular examples, the heterologous TCR is reactive to a neoantigen (such as a neoantigen expressed by a tumor from a subject). In one example, the TCR is reactive to a mutant KRAS protein (e.g., KRASG12D).

Also provided are compositions including the disclosed modified T cells and a pharmaceutically acceptable carrier.

Further disclosed are methods of preparing the disclosed modified T cells. In some embodiments, the methods include transducing T cells (such as tumor-reactive T cells) with a nucleic acid encoding a CD40 protein or chimeric CD40 polypeptide and culturing the transduced T cells to produce the modified T cells. In some embodiments, the methods further include transducing the T cells with a nucleic acid encoding a heterologous T cell receptor, a CAR, or both. In some examples, the T cells are from a subject with cancer.

Provided herein are methods of treating a subject with cancer with the disclosed modified T cells. In some embodiments, the methods include administering an effective amount of the modified T cells or a composition including the modified T cells to a subject with cancer. In some examples, the subject has colorectal or pancreatic cancer. In some examples, the methods further include administering to the subject a CD40 agonist (such as CD40L or an agonistic anti-CD40 antibody). In some examples, the modified T cells are autologous to the subject. The methods may further include administering one or more additional therapies to the subject (for example, one or more of surgery, radiation, a chemotherapeutic agent, a checkpoint inhibitor, or a combination of two or more thereof).

Chimeric CD40 proteins are also provided herein. In some embodiments, the chimeric CD40 protein includes an extracellular CD40 domain, a transmembrane domain, and one or more heterologous intracellular domains. In some examples, the heterologous intracellular domain (or portion thereof) is from a member of the CD28 family (such as CD28 or ICOS), CD2, CD4, CD8, a cytokine receptor (such as CSF2RB, GMCSFRa, IL3Ra, IL2Rb, IL2Rg (common gamma chain), IL9R, IL7Ra, or TSLPR), a member of the TNFR family (such as 4-1BB, OX40, CD27, CD30, GITR, or TNFR2), a TNF ligand (such as RANKL, LIGHT, FasL, 41BBL, CD30L, CD27L, OX40L, TWEAK, BAFF, TRAIL, EDA, or GITRL), a member of the TLR family (such as TLR1, TLR2, TLR3, TLR4, TLR5, TLR6, TLR7, TLR8, TLR9, or TLR10), a member of the receptor tyrosine kinase (RTK) family (such as IGF1R, EGFR, TRKA, TRKB, TRKC, MUSK, MET, AXL, TIE2, EPHA (EPHA1, EPHA3, EPHA7, or EPHA8), EPHB (EPHB1, EPHB2, EPHB3, or EPHB6), DDR1, ROS1, LTK, or ALK), a member of the integrins family (such as CD11A or CD18 (beta-2 integrin)), or a member of the KIR family (such as NKG2D, CD94, NKP46, NKP30, NKP44, NKP80, KIR2DS1, CLEC2D, or KIR3DS1). In some embodiments, the transmembrane domain is a CD40 transmembrane domain. In other embodiments, the transmembrane domain is a heterologous transmembrane domain, for example, a transmembrane domain from the same protein as the heterologous intracellular domain. Nucleic acids encoding chimeric CD40 proteins are also provided.

The foregoing and other features of the disclosure will become more apparent from the following detailed description, which proceeds with reference to the accompanying figures.

### BRIEF DESCRIPTION OF THE DRAWINGS

FIG. 1 shows ectopic co-expression of wild-type CD40 and a neoantigen-reactive T-cell receptor (TCR) in human T cells. Peripheral blood mononuclear cells were stimulated with OKT3 (50 ng/mL) and IL-2 (300 IU/ml) for two days and then transduced with a retroviral vector encoding a TCR recognizing an HLA-C*08:02-restricted KRAS-G12D neoantigen (left) or a retroviral vector encoding the same TCR as well as the wild-type CD40 receptor (right). Flow cytometry was used to measure TCR transduction efficiency using a fluorochrome-conjugated anti-mouse TCRb constant region antibody (mTCRB) and CD40 expression was measured using a fluorochrome conjugated anti-human CD40 antibody. The KRAS-G12D-neoantigen-reactive TCR contains mouse TCR constant regions to promote pairing of the introduced TCR as well as to facilitate detection of the transduced T cells.

FIGS. 2A-2D show that ectopic expression of CD40 enhances cytokine production and proliferation of neoantigen-reactive T cells against a cancer cell line. T cells transduced to express either the HLA-C*08:02-restricted KRAS-G12D reactive TCR alone (TCR) or the same TCR and CD40 (TCR+CD40) were cocultured for 5 hours with the KRAS-G12D-positive and HLA-C*08:02-positive HPAC pancreatic cancer cell line in the presence or absence of an agonistic antibody against CD40 (10 μg/ml, CDX-1140). Intracellular cytokine staining was performed and flow cytometric analysis was used to detect the presence of IFN-g (FIG. 2A), TNF (FIG. 2B), and the degranulation marker CD107a (FIG. 2C). The T cells described above were cocultured with the HPAC cell line for 3 days with EdU pulsing on the final day, and cell proliferation was measured by intracellular flow cytometry detection of EdU (FIG. 2D). Data were gated on CD8+mTCRb+ (transduced) T cells.

FIGS. 3A and 3B show that ectopic expression of CD40 enhanced in vitro tumor cell killing by neoantigen-reactive T cells. T cells transduced to express either the HLA-C*08:02-restricted KRAS-G12D reactive TCR alone (TCR) or the same TCR and CD40 (TCR+CD40) were cocultured for 3 days with the KRAS-G12D-positive and HLA-C*08:02-positive HPAC pancreatic cancer cell line stably expressing GFP in the absence (FIG. 3A) or presence (FIG. 3B) of an agonistic antibody against CD40 (10 μg/ml, CDX-1140). Live cell imaging and GFP fluorescence quantitation was measured over the 3 days using a cell imager. ns, not significant by paired t-test.

FIGS. 4A and 4B illustrate that ectopic expression of CD40-CD28 chimeric receptors enhances cytokine production of neoantigen-reactive T cells against a cancer cell line. T cells transduced to express either the HLA-C*08:02-restricted KRAS-G12D reactive TCR alone (TCR) or the same TCR and CD40-CD28 chimeric receptors (expressing either the transmembrane domain, tm, derived from CD40 or CD28) were cocultured for 5 hours with the KRAS-G12D-positive MDA-Panc48 pancreatic cancer cell line that was transduced to express HLA-C*08:02, in the presence or absence of an agonistic antibody against CD40 (10 μg/ml, CDX-1140). Intracellular cytokine staining was performed and flow cytometric analysis was used to detect the presence of IFN-g (FIG. 4A) or TNF (FIG. 4B). Data were gated on CD8+mTCRb+ (transduced) T cells.

FIGS. 5A-5C illustrate that stimulation of T cells expressing chimeric CD40-cytokine receptors with agonistic CD40 antibodies can activate STAT5 and enhance proliferation of neoantigen-reactive T cells against a cancer cell line. T cells transduced to express either the HLA-C*08:02-restricted KRAS-G12D reactive TCR alone (TCR) or the same TCR co-transduced to express CD40-GMSCFRa (GM) and CD40-CSF2Rb (Bc), or CD40-IL2Rb (IL2b) and CD40-common gamma chain (gc) chimeric receptors (expressing either the transmembrane domain, tm, derived from CD40 or the corresponding cytokine receptors) were stimulated for 30 minutes with (bottom) or without (top) agonistic antibody against CD40 (10 μg/ml, CDX-1140) and phospho-STAT5 was measured by intracellular flow cytometry (FIG. 5A). T cells described above were cocultured for 3 days with the KRAS-G12D-positive and HLA-C*08:02 positive HPAC cell line, in the presence or absence of an agonistic antibody against CD40 (10 μg/ml, CDX-1140) with EdU pulsed on the final day. EdU was detected by intracellular staining and flow cytometry (FIG. 5B) and (FIG. 5C). Data were gated on CD8+mTCRb+ (transduced) T cells.

FIGS. 6A-6C show that ectopic expression of CD40 chimeric receptors enhanced in vitro tumor cell killing by neoantigen-reactive T cells. FIG. 6A shows enhanced cell death of KRAS-G12D-positive and HLA-C*08:02-positive HPAC pancreatic cancer cell line in the presence of T cells co-transduced to express the HLA-C*08:02-restricted KRAS-G12D reactive TCR and CD40-CSF2Rb (Bc) (expressing the transmembrane domain, tm, derived from CSF2Rb) and CD40-GMCSFRa (GM) (expressing the transmembrane domain, tm, derived from GMCSFRa) in the presence of an agonistic antibody against CD40. FIG. 6B shows enhanced cell death of KRAS-G12D-positive and HLA-C*08:02-positive HPAC pancreatic cancer cell line in the presence of T cells co-transduced to express the HLA-C*08:02-restricted KRAS-G12D reactive TCR and CD40-CD28_YMFM chimeric receptors (expressing the transmembrane domain, tm, derived from CD28) in the presence of an agonistic antibody against CD40. FIG. 6C shows enhanced cell death of the KRAS-G12D-positive and HLA-C*08:02-positive MDA-Panc48 pancreatic adenocarcinoma cell line in the presence of T cells co-transduced to express the HLA-C*08:02-restricted KRAS-G12D reactive TCR and CD40-IGF1R (expressing the transmembrane domain, tm, derived from IGF1R) chimeric receptor in the presence of an agonistic antibody against CD40.

FIGS. 7A and 7B show that ectopic expression of CD40 chimeric receptors enhanced in vitro tumor cell killing by neoantigen-reactive T cells in the absence of anti-CD40 antibody. T cells transduced to express the HLA-C*08:02-restricted KRAS-G12D reactive TCR and CD40-4-1BB (expressing the transmembrane domain, tm, derived from 4-1BB) (FIG. 7A) or CD40-TLR4 (expressing the transmembrane domain, tm, derived from TLR4) (FIG. 7B) were cocultured for 5 days with KRAS-G12D-positive and HLA-C*08:02-positive HPAC pancreatic cancer cell line stably expressing GFP in the absence or presence of an agonistic antibody against CD40.

FIGS. 8A-8C show IFN-g (FIG. 8A), TNF-α (FIG. 8B), and CD107a (FIG. 8C) production by neoantigen-reactive T cells transduced to express the HLA-C*08:02-restricted KRAS-G12D reactive TCR and CD40-IGF1R (expressing the transmembrane domain, tm, derived from IGF1R) in the presence of KRAS-G12D-positive and HLA-C*08:02-positive HPAC pancreatic cancer cell line in the presence or absence of an agonistic antibody against CD40.

### SEQUENCE LISTING

Any nucleic acid and amino acid sequences listed herein or in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is an exemplary amino acid sequence of a human CD40 protein:

MVRLPLQCVLWGCLLTAVHPEPPTACREKQYLINSQCCSLCQPGQKLVSDCTEFTETECLPCGESEFL
DTWNRETHCHQHKYCDPNLGLRVQQKGTSETDTICTCEEGWHCTSEACESCVLHRSCSPGFGVKQIAT
GVSDTICEPCPVGFFSNVSSAFEKCHPWTSCETKDLVVQQAGTNKTDVVCGPQDRLRALVVIPIIFGI
LFAILLVLVFIKKVAKKPTNKAPHPKQEPQEINFPDDLPGSNTAAPVQETLHGCQPVTQEDGKESRIS
VQERQ

SEQ ID NO: 2 is an exemplary amino acid sequence of a human CD40 extracellular domain:

MVRLPLQCVLWGCLLTAVHPEPPTACREKQYLINSQCCSLCQPGQKLVSDCTEFTETECLPCGESEFL
DTWNRETHCHQHKYCDPNLGLRVQQKGTSETDTICTCEEGWHCTSEACESCVLHRSCSPGFGVKQIAT
GVSDTICEPCPVGFFSNVSSAFEKCHPWTSCETKDLVVQQAGTNKTDVVCGPQDRLR

SEQ ID NO: 3 is an exemplary amino acid sequence of human CD40 protein transmembrane domain:

ALVVIPIIFGILFAILLVLVFI

SEQ ID NO: 4 is an exemplary amino acid sequence of an intracellular signaling domain of human CD40 protein:

KKVAKKPTNKAPHPKQEPQEINFPDDLPGSNTAAPVQETLHGCQPVTQEDGKESRISVQERQ

SEQ ID NO: 5 is an exemplary amino acid sequence of an intracellular signaling domain of human CD28:

RSKRSRLLHSDYMNMTPRRPGPTRKHYQPYAPPRDFAAYRS

SEQ ID NO: 6 is an exemplary amino acid sequence of an intracellular signaling domain of human 4-1BB:

KRGRKKLLYIFKQPFMRPVQTTQEEDGCSCRFPEEEEGGCEL

SEQ ID NO: 7 is an exemplary amino acid sequence of an intracellular signaling domain of human OX40:

ALYLLRRDQRLPPDAHKPPGGGSFRTPIQEEQADAHSTLAKI

SEQ ID NO: 8 is an exemplary amino acid sequence of an intracellular signaling domain of human CD27:

QRRKYRSNKGESPVEPAEPCHYSCPREEEGSTIPIQEDYRKPEPACSP

SEQ ID NO: 9 is an exemplary amino acid sequence of an intracellular signaling domain of human CD30:

CHRRACRKRIRQKLHLCYPVQTSQPKLELVDSRPRRSSTQLRSGASVTEPVAEERGLMSQPLMETCHS
VGAAYLESLPLQDASPAGGPSSPRDLPEPRVSTEHTNNKIEKIYIMKADTVIVGTVKAELPEGRGLAG
PAEPELEEELEADHTPHYPEQETEPPLGSCSDVMLSVEEEGKEDPLPTAASGK

SEQ ID NO: 10 is an exemplary amino acid sequence of an intracellular signaling domain of human GITR:

QLGLHIWQLRSQCMWPRETQLLLEVPPSTEDARSCQFPEEERGERSAEEKGRLGDLWV

SEQ ID NO: 11 is an exemplary amino acid sequence of an intracellular signaling domain of human TNFR2:

KKKPLCLQREAKVPHLPADKARGTQGPEQQHLLITAPSSSSSSLESSASALDRRAPTRNQPQAPGVEA
SGAGEARASTGSSDSSPGGHGTQVNVTCIVNVCSSSDHSSQCSSQASSTMGDTDSSPSESPKDEQVPF
SKEECAFRSQLETPETLLGSTEEKPLPLGVPDAGMKS

SEQ ID NO: 12 is an exemplary amino acid sequence of an intracellular signaling domain of human RANKL:

MRRASRDYTKYLRGSEEMGGGPGAPHEGPLHAPPPPAPHQPPAASRS

SEQ ID NO: 13 is an exemplary amino acid sequence of an intracellular signaling domain of

MEESVVRPSVFVVDGQTDTPFTRLGRSHRRQSCSVAR

SEQ ID NO: 14 is the amino acid sequence of an exemplary CD40-CD28tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRFWVLVVVG
GVLACYSLLVTVAFIIFWVRSKRSRLLHSDYMNMTPRRPGPTRKHYQPYAPPRDFAAYRS

SEQ ID NO: 15 is an exemplary codon-optimized nucleic acid sequence of a CD40-CD28tm chimera:

ATGGTGCGGCTGCCTCTGCAGTGCGTGCTGTGGGGATGTCTGCTGACCGCAGTGCACCCAGAGCCAC
CAACAGCCTGTAGAGAGAAGCAGTACCTGATCAATTCTCAGTGCTGTAGCCTGTGCCAGCCAGGCCA
GAAGCTGGTGTCCGATTGTACCGAGTTCACCGAGACAGAGTGCCTGCCCTGTGGCGAGTCTGAGTTT
CTGGATACCTGGAACCGGGAGACACACTGCCACCAGCACAAGTATTGTGACCCTAATCTGGGCCTGA
GAGTGCAGCAGAAGGGCACCTCCGAGACCGACACAATCTGCACATGTGAGGAGGGATGGCACTGCAC
CAGCGAGGCATGCGAGTCCTGCGTGCTGCACAGATCCTGTTCTCCAGGCTTCGGCGTGAAGCAGATC
GCCACCGGCGTGTCTGATACAATCTGCGAGCCATGTCCCGTGGGCTTCTTTAGCAACGTGTCCTCTG
CCTTTGAGAAGTGCCACCCCTGGACCAGCTGTGAGACAAAGGACCTGGTGGTGCAGCAGGCCGGCAC
CAATAAGACAGATGTGGTGTGCGGACCACAGGACAGGCTGAGGTTCTGGGTGCTGGTGGTGGTGGGA
GGCGTGCTGGCCTGTTACTCTCTGCTGGTGACCGTGGCCTTCATCATCTTTTGGGTGAGGTCCAAGC
GGTCTAGACTGCTGCACAGCGATTATATGAACATGACCCCACGGAGACCTGGCCCAACACGCAAGCA
CTACCAGCCCTATGCCCCTCCAAGGGACTTTGCCGCCTACCGCAGC

SEQ ID NO: 16 is the amino acid sequence of an exemplary CD40tm-CD28 chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRALVVIPII
FGILFAILLVLVFIRSKRSRLLHSDYMNMTPRRPGPTRKHYQPYAPPRDFAAYRS

SEQ ID NO: 17 is an exemplary codon-optimized nucleic acid sequence of a CD40tm-CD28 chimera:

ATGGTGCGGCTGCCTCTGCAGTGCGTGCTGTGGGGATGTCTGCTGACCGCAGTGCACCCAGAGCCAC
CAACAGCCTGTAGAGAGAAGCAGTACCTGATCAATTCTCAGTGCTGTAGCCTGTGCCAGCCAGGCCA
GAAGCTGGTGTCCGATTGTACCGAGTTCACCGAGACAGAGTGCCTGCCCTGTGGCGAGTCTGAGTTT
CTGGATACCTGGAACCGGGAGACACACTGCCACCAGCACAAGTATTGTGACCCTAATCTGGGCCTGA
GAGTGCAGCAGAAGGGCACCTCCGAGACCGACACAATCTGCACATGTGAGGAGGGATGGCACTGCAC
CAGCGAGGCATGCGAGTCCTGCGTGCTGCACAGATCCTGTTCTCCAGGCTTCGGCGTGAAGCAGATC
GCCACCGGCGTGTCTGATACAATCTGCGAGCCATGTCCCGTGGGCTTCTTTAGCAACGTGTCCTCTG
CCTTTGAGAAGTGCCACCCCTGGACCAGCTGTGAGACAAAGGACCTGGTGGTGCAGCAGGCCGGCAC
CAATAAGACAGATGTGGTGTGCGGACCACAGGACAGGCTGAGGGCCCTGGTGGTCATCCCCATCATC
TTCGGCATCCTGTTTGCCATCCTGCTGGTGCTGGTGTTCATCAGGTCCAAGCGGTCTAGACTGCTGC
ACAGCGATTATATGAACATGACCCCACGGAGACCTGGCCCAACACGCAAGCACTACCAGCCCTATGC
CCCTCCAAGGGACTTTGCCGCCTACCGCAGC

SEQ ID NO: 18 is the amino acid sequence of an exemplary CD40tm-GMCSFRA chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRALVVIPII
FGILFAILLVLVFIKRFLRIQRLFPPVPQIKDKLNDNHEVEDEIIWEEFTPEEGKGYREEVLTVKEI
T

SEQ ID NO: 19 is an exemplary codon-optimized nucleic acid sequence of a CD40tm-GMCSFRA chimera:

ATGGTGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAGCCCTGGTGGTCATCCCAATCATC
TTCGGCATCCTGTTTGCCATCCTGCTGGTGCTGGTGTTCATCAAGCGGTTCCTGAGAATCCAGAGGC
TGTTTCCCCCTGTGCCACAGATCAAGGACAAGCTGAACGATAATCACGAGGTGGAGGACGAGATCAT
CTGGGAGGAGTTCACCCCAGAGGAGGGC

SEQ ID NO: 20 is the amino acid sequence of an exemplary CD40-GMCSFRAtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRNLGSVYIY
VLLIVGTLVCGIVLGFLFKRFLRIQRLFPPVPQIKDKLNDNHEVEDEIIWEEFTPEEGKGYREEVLT
VKEIT

SEQ ID NO: 21 is an exemplary codon-optimized nucleic acid sequence of a CD40-GMCSFRAtm chimera:

ATGGTGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAAACCTGGGCAGCGTGTACATCTAT
GTGCTGCTGATCGTGGGCACCCTGGTGTGCGGAATCGTGCTGGGCTTCCTGTTTAAGCGGTTCCTGA
GAATCCAGAGGCTGTTTCCCCCTGTGCCACAGATCAAGGACAAGCTGAACGATAATCACGAGGTGGA
GGACGAGATCATCTGGGAGGAGTTCACCCCAGAGGAGGGCAAGGGCTACCGGGAGGAGGTGCTGACC
GTGAAGGAGATCACA

SEQ ID NO: 22 is the amino acid sequence of an exemplary CD40tm-CSF2RB chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRALVVIPII
FGILFAILLVLVFIRFCGIYGYRLRRKWEEKIPNPSKSHLFQNGSAELWPPGSMSAFTSGSPPHQGP
WGSRFPELEGVFPVGFGDSEVSPLTIEDPKHVCDPPSGPDTTPAASDLPTEQPPSPQPGPPAASHTP
EKQASSFDFNGPYLGPPHSRSLPDILGQPEPPQEGGSQKSPPPGSLEYLCLPAGGQVQLVPLAQAMG
PGQAVEVERRPSQGAAGSPSLESGGGPAPPALGPRVGGQDQKDSPVAIPMSSGDTEDPGVASGYVSS
ADLVFTPNSGASSVSLVPSLGLPSDQTPSLCPGLASGPPGAPGPVKSGFEGYVELPPIEGRSPRSPR
NNPVPPEAKSPVLNPGERPADVSPTSPQPEGLLVLQQVGDYCFLPGLGPGPLSLRSKPSSPGPGPEI
KNLDQAFQVKKPPGQAVPQVPVIQLFKALKQQDYLSLPPWEVNKPGEVC

SEQ ID NO: 23 is an exemplary codon-optimized nucleic acid sequence of a CD40tm-CSF2RB chimera:

ATGGTGAGGCTGCCACTGCAGTGCGTGCTGTGGGGATGTCTGCTGACCGCAGTGCACCCAGAGCCAC
CAACAGCCTGTCGCGAGAAGCAGTACCTGATCAATTCTCAGTGCTGTAGCCTGTGCCAGCCCGGCCA
GAAGCTGGTGTCCGATTGTACCGAGTTCACCGAGACAGAGTGCCTGCCTTGTGGCGAGTCTGAGTTT
CTGGATACCTGGAACCGGGAGACACACTGCCACCAGCACAAGTATTGTGACCCCAATCTGGGCCTGA
GAGTGCAGCAGAAGGGCACCTCCGAGACCGACACAATCTGCACATGTGAGGAGGGATGGCACTGCAC
CAGCGAGGCATGCGAGTCCTGCGTGCTGCACCGCTCCTGTTCTCCCGGCTTCGGCGTGAAGCAGATC
GCCACCGGCGTGTCTGATACAATCTGCGAGCCCTGTCCTGTGGGCTTCTTTAGCAACGTGTCCTCTG
CCTTTGAGAAGTGCCACCCTTGGACCAGCTGTGAGACAAAGGACCTGGTGGTGCAGCAGGCCGGCAC
CAATAAGACAGATGTGGTGTGCGGACCTCAGGACAGGCTGAGAGCCCTGGTGGTCATCCCAATCATC
TTCGGCATCCTGTTTGCCATCCTGCTGGTGCTGGTGTTCATCCGCTTCTGCGGCATCTACGGCTATC
GGCTGCGGAGAAAGTGGGAGGAGAAGATCCCAAACCCCTCCAAGTCTCACCTGTTTCAGAATGGCTC
TGCCGAGCTGTGGCCCCCTGGCAGCATGTCCGCCTTCACCTCTGGCAGCCCACCACACCAGGGACCC
TGGGGCTCCAGATTTCCTGAGCTGGAGGGCGTGTTCCCAGTGGGATTTGGCGATTCTGAGGTGAGCC
CACTGACAATCGAGGACCCCAAGCACGTGTGCGATCCTCCATCCGGACCTGACACCACACCAGCAGC
CTCTGACCTGCCAACCGAGCAGCCACCTAGCCCTCAGCCAGGCCCACCAGCAGCCTCCCACACACCA
GAGAAGCAGGCCAGCAGCTTCGACTTCAACGGACCTTACCTGGGACCTCCACACTCCCGCTCTCTGC
CTGACATCCTGGGACAGCCAGAGCCACCTCAGGAGGGAGGCTCCCAGAAGTCTCCACCCCCTGGCAG
CCTGGAGTATCTGTGCCTGCCAGCAGGAGGACAGGTGCAGCTGGTGCCCCTGGCCCAGGCTATGGGC
CCCGGCCAGGCCGTGGAGGTGGAGAGGCGCCCTTCTCAGGGCGCCGCCGGCAGCCCATCCCTGGAGA
GCGGAGGAGGACCAGCACCACCCGCCCTGGGCCCCAGAGTGGGCGGCCAGGACCAGAAGGATTCCCC
CGTGGCCATCCCTATGTCTAGCGGCGACACCGAGGACCCCGGCGTGGCCAGCGGCTACGTGTCCTCT
GCCGATCTGGTGTTCACACCAAACTCCGGAGCCAGCTCCGTGAGCCTGGTGCCTTCCCTGGGACTGC
CATCTGACCAGACCCCCAGCCTGTGCCCCGGCCTGGCCAGCGGACCTCCAGGCGCCCCCGGCCCTGT
GAAGTCTGGCTTTGAGGGCTATGTGGAGCTGCCACCTATCGAGGGCCGGAGCCCAAGATCCCCTAGG
AACAATCCCGTGCCACCAGAGGCAAAGAGCCCTGTGCTGAATCCAGGCGAGAGACCAGCAGACGTGA
GCCCTACAAGCCCACAGCCAGAGGGACTGCTGGTGCTGCAGCAAGTGGGCGACTACTGCTTCCTGCC
AGGCCTGGGCCCTGGCCCACTGTCCCTGAGGTCTAAGCCCTCTAGCCCCGGCCCTGGCCCAGAGATC
AAGAACCTGGATCAGGCCTTCCAGGTGAAGAAGCCTCCAGGCCAGGCCGTGCCACAGGTGCCCGTGA
TCCAGCTGTTTAAGGCCCTGAAGCAGCAGGACTATCTGAGCCTGCCCCCTTGGGAAGTGAATAAGCC
TGGCGAGGTGTGC

SEQ ID NO: 24 is the amino acid sequence of an exemplary CD40-CSF2RBtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRVLALIVIF
LTIAVLLALRFCGIYGYRLRRKWEEKIPNPSKSHLFQNGSAELWPPGSMSAFTSGSPPHQGPWGSRF
PELEGVFPVGFGDSEVSPLTIEDPKHVCDPPSGPDTTPAASDLPTEQPPSPQPGPPAASHTPEKQAS
SFDFNGPYLGPPHSRSLPDILGQPEPPQEGGSQKSPPPGSLEYLCLPAGGQVQLVPLAQAMGPGQAV
EVERRPSQGAAGSPSLESGGGPAPPALGPRVGGQDQKDSPVAIPMSSGDTEDPGVASGYVSSADLVF
TPNSGASSVSLVPSLGLPSDQTPSLCPGLASGPPGAPGPVKSGFEGYVELPPIEGRSPRSPRNNPVP
PEAKSPVLNPGERPADVSPTSPQPEGLLVLQQVGDYCFLPGLGPGPLSLRSKPSSPGPGPEIKNLDQ
AFQVKKPPGQAVPQVPVIQLFKALKQQDYLSLPPWEVNKPGEVC

SEQ ID NO: 25 is an exemplary codon-optimized nucleic acid sequence of a CD40-CSF2RBtm chimera:

ATGGTGAGGCTGCCACTGCAGTGCGTGCTGTGGGGATGTCTGCTGACCGCAGTGCACCCAGAGCCAC
CAACAGCCTGTCGCGAGAAGCAGTACCTGATCAATTCTCAGTGCTGTAGCCTGTGCCAGCCCGGCCA
GAAGCTGGTGTCCGATTGTACCGAGTTCACCGAGACAGAGTGCCTGCCTTGTGGCGAGTCTGAGTTT
CTGGATACCTGGAACCGGGAGACACACTGCCACCAGCACAAGTATTGTGACCCCAATCTGGGCCTGA
GAGTGCAGCAGAAGGGCACCTCCGAGACCGACACAATCTGCACATGTGAGGAGGGATGGCACTGCAC
CAGCGAGGCATGCGAGTCCTGCGTGCTGCACCGCTCCTGTTCTCCCGGCTTCGGCGTGAAGCAGATC
GCCACCGGCGTGTCTGATACAATCTGCGAGCCCTGTCCTGTGGGCTTCTTTAGCAACGTGTCCTCTG
CCTTTGAGAAGTGCCACCCTTGGACCAGCTGTGAGACAAAGGACCTGGTGGTGCAGCAGGCCGGCAC
CAATAAGACAGATGTGGTGTGCGGACCTCAGGACAGGCTGAGAGTGCTGGCCCTGATCGTGATCTTC
CTGACCATCGCCGTGCTGCTGGCCCTGCGCTTCTGCGGCATCTACGGCTATCGGCTGCGGAGAAAGT
GGGAGGAGAAGATCCCAAACCCCTCCAAGTCTCACCTGTTTCAGAATGGCTCTGCCGAGCTGTGGCC
CCCTGGCAGCATGTCCGCCTTCACCTCTGGCAGCCCACCACACCAGGGACCCTGGGGCTCCAGATTT
CCTGAGCTGGAGGGCGTGTTCCCAGTGGGATTTGGCGATTCTGAGGTGAGCCCACTGACAATCGAGG
ACCCCAAGCACGTGTGCGATCCTCCATCCGGACCTGACACCACACCAGCAGCCTCTGACCTGCCAAC
CGAGCAGCCACCTAGCCCTCAGCCAGGCCCACCAGCAGCCTCCCACACACCAGAGAAGCAGGCCAGC
AGCTTCGACTTCAACGGACCTTACCTGGGACCTCCACACTCCCGCTCTCTGCCTGACATCCTGGGAC
AGCCAGAGCCACCTCAGGAGGGAGGCTCCCAGAAGTCTCCACCCCCTGGCAGCCTGGAGTATCTGTG
CCTGCCAGCAGGAGGACAGGTGCAGCTGGTGCCCCTGGCCCAGGCTATGGGCCCCGGCCAGGCCGTG
GAGGTGGAGAGGCGCCCTTCTCAGGGCGCCGCCGGCAGCCCATCCCTGGAGAGCGGAGGAGGACCAG
CACCACCCGCCCTGGGCCCCAGAGTGGGCGGCCAGGACCAGAAGGATTCCCCCGTGGCCATCCCTAT
GTCTAGCGGCGACACCGAGGACCCCGGCGTGGCCAGCGGCTACGTGTCCTCTGCCGATCTGGTGTTC
ACACCAAACTCCGGAGCCAGCTCCGTGAGCCTGGTGCCTTCCCTGGGACTGCCATCTGACCAGACCC
CCAGCCTGTGCCCCGGCCTGGCCAGCGGACCTCCAGGCGCCCCCGGCCCTGTGAAGTCTGGCTTTGA
GGGCTATGTGGAGCTGCCACCTATCGAGGGCCGGAGCCCAAGATCCCCTAGGAACAATCCCGTGCCA
CCAGAGGCAAAGAGCCCTGTGCTGAATCCAGGCGAGAGACCAGCAGACGTGAGCCCTACAAGCCCAC
AGCCAGAGGGACTGCTGGTGCTGCAGCAAGTGGGCGACTACTGCTTCCTGCCAGGCCTGGGCCCTGG
CCCACTGTCCCTGAGGTCTAAGCCCTCTAGCCCCGGCCCTGGCCCAGAGATCAAGAACCTGGATCAG
GCCTTCCAGGTGAAGAAGCCTCCAGGCCAGGCCGTGCCACAGGTGCCCGTGATCCAGCTGTTTAAGG
CCCTGAAGCAGCAGGACTATCTGAGCCTGCCCCCTTGGGAAGTGAATAAGCCTGGCGAGGTGTGC

SEQ ID NO: 26 is the amino acid sequence of an exemplary CD40tm-IL2RB chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRALVVIPII
FGILFAILLVLVFINCRNTGPWLKKVLKCNTPDPSKFFSQLSSEHGGDVQKWLSSPFPSSSFSPGGL
APEISPLEVLERDKVTQLLLQQDKVPEPASLSSNHSLTSCFTNQGYFFFHLPDALEIEACQVYFTYD
PYSEEDPDEGVAGAPTGSSPQPLQPLSGEDDAYCTFPSRDDLLLFSPSLLGGPSPPSTAPGGSGAGE
ERMPPSLQERVPRDWDPQPLGPPTPGVPDLVDFQPPPELVLREAGEEVPDAGPREGVSFPWSRPPGQ
GEFRALNARLPLNTDAYLSLQELQGQDPTHL

SEQ ID NO: 27 is an exemplary codon-optimized nucleic acid sequence of a CD40tm-IL2RB chimera:

ATGGTGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAGCCCTGGTGGTCATCCCAATCATC
TTCGGCATCCTGTTTGCCATCCTGCTGGTGCTGGTGTTCATCAACTGCCGGAATACCGGCCCTTGGC
TGAAGAAGGTGCTGAAGTGTAACACACCAGACCCCTCTAAGTTCTTTAGCCAGCTGAGCAGCGAGCA
CGGAGGCGATGTGCAGAAGTGGCTGTCTAGCCCTTTTCCATCCTCTAGCTTCAGCCCAGGCGGCCTG
GCCCCAGAGATCTCCCCCCTGGAGGTGCTGGAGAGGGACAAGGTGACCCAGCTGCTGCTGCAGCAGG
ATAAGGTGCCCGAGCCTGCCTCCCTGTCCTCTAACCACTCCCTGACCTCTTGCTTTACAAATCAGGG
CTATTTCTTTTTCCACCTGCCTGACGCCCTGGAGATCGAGGCCTGTCAGGTGTACTTCACCTATGAT
CCCTACAGCGAGGAGGACCCTGATGAGGGAGTGGCAGGCGCCCCTACAGGCAGCTCCCCACAGCCAC
TGCAGCCACTGTCCGGAGAGGACGATGCCTACTGCACCTTTCCCTCTAGGGACGATCTGCTGCTGTT
CTCTCCAAGCCTGCTGGGAGGACCAAGCCCACCTTCCACAGCCCCAGGCGGCTCTGGCGCCGGCGAG
GAGCGCATGCCACCCAGCCTGCAGGAGCGGGTGCCCAGAGACTGGGACCCCCAGCCACTGGGACCTC
CAACACCCGGCGTGCCTGACCTGGTGGATTTTCAGCCCCCTCCAGAGCTGGTGCTGAGAGAGGCAGG
AGAGGAGGTGCCTGACGCAGGACCACGGGAGGGCGTGTCCTTTCCCTGGAGCCGGCCCCCCGGCCAG
GGAGAGTTCAGGGCCCTGAACGCCCGCCTGCCCCTGAATACCGACGCCTATCTGTCCCTGCAGGAGC
TGCAGGGACAGGACCCCACACACCTG

SEQ ID NO: 28 is the amino acid sequence of an exemplary CD40-IL2RBtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRIPWLGHLL
VGLSGAFGFIILVYLLINCRNTGPWLKKVLKCNTPDPSKFFSQLSSEHGGDVQKWLSSPFPSSSFSP
GGLAPEISPLEVLERDKVTQLLLQQDKVPEPASLSSNHSLTSCFTNQGYFFFHLPDALEIEACQVYF
TYDPYSEEDPDEGVAGAPTGSSPQPLQPLSGEDDAYCTFPSRDDLLLFSPSLLGGPSPPSTAPGGSG
AGEERMPPSLQERVPRDWDPQPLGPPTPGVPDLVDFQPPPELVLREAGEEVPDAGPREGVSFPWSRP
PGQGEFRALNARLPLNTDAYLSLQELQGQDPTHL

SEQ ID NO: 29 is an exemplary codon-optimized nucleic acid sequence of a CD40-IL2RBtm chimera:

ATGGTGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATCCCCTGGCTGGGACACCTGCTG
GTGGGACTGAGCGGAGCCTTCGGCTTTATCATCCTGGTGTACCTGCTGATCAACTGCCGGAATACCG
GCCCTTGGCTGAAGAAGGTGCTGAAGTGTAACACACCAGACCCCTCTAAGTTCTTTAGCCAGCTGAG
CAGCGAGCACGGAGGCGATGTGCAGAAGTGGCTGTCTAGCCCTTTTCCATCCTCTAGCTTCAGCCCA
GGCGGCCTGGCCCCAGAGATCTCCCCCCTGGAGGTGCTGGAGAGGGACAAGGTGACCCAGCTGCTGC
TGCAGCAGGATAAGGTGCCCGAGCCTGCCTCCCTGTCCTCTAACCACTCCCTGACCTCTTGCTTTAC
AAATCAGGGCTATTTCTTTTTCCACCTGCCTGACGCCCTGGAGATCGAGGCCTGTCAGGTGTACTTC
ACCTATGATCCCTACAGCGAGGAGGACCCTGATGAGGGAGTGGCAGGCGCCCCTACAGGCAGCTCCC
CACAGCCACTGCAGCCACTGTCCGGAGAGGACGATGCCTACTGCACCTTTCCCTCTAGGGACGATCT
GCTGCTGTTCTCTCCAAGCCTGCTGGGAGGACCAAGCCCACCTTCCACAGCCCCAGGCGGCTCTGGC
GCCGGCGAGGAGCGCATGCCACCCAGCCTGCAGGAGCGGGTGCCCAGAGACTGGGACCCCCAGCCAC
TGGGACCTCCAACACCCGGCGTGCCTGACCTGGTGGATTTTCAGCCCCCTCCAGAGCTGGTGCTGAG
AGAGGCAGGAGAGGAGGTGCCTGACGCAGGACCACGGGAGGGCGTGTCCTTTCCCTGGAGCCGGCCC
CCCGGCCAGGGAGAGTTCAGGGCCCTGAACGCCCGCCTGCCCCTGAATACCGACGCCTATCTGTCCC
TGCAGGAGCTGCAGGGACAGGACCCCACACACCTG

SEQ ID NO: 30 is the amino acid sequence of an exemplary CD40tm-IL2RG chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRALVVIPII
FGILFAILLVLVFIERTMPRIPTLKNLEDLVTEYHGNFSAWSGVSKGLAESLQPDYSERLCLVSEIP
PKGGALGEGPGASPCNQHSPYWAPPCYTLKPET

SEQ ID NO: 31 is an exemplary codon-optimized nucleic acid sequence of a CD40tm-IL2RG chimera:

ATGGTGAGGCTGCCACTGCAGTGCGTGCTGTGGGGATGTCTGCTGACCGCAGTGCACCCAGAGCCAC
CAACAGCCTGTCGCGAGAAGCAGTACCTGATCAATTCTCAGTGCTGTAGCCTGTGCCAGCCCGGCCA
GAAGCTGGTGTCCGATTGTACCGAGTTCACCGAGACAGAGTGCCTGCCTTGTGGCGAGTCTGAGTTT
CTGGATACCTGGAACCGGGAGACACACTGCCACCAGCACAAGTATTGTGACCCCAATCTGGGCCTGA
GAGTGCAGCAGAAGGGCACCTCCGAGACCGACACAATCTGCACATGTGAGGAGGGATGGCACTGCAC
CAGCGAGGCATGCGAGTCCTGCGTGCTGCACCGCTCCTGTTCTCCCGGCTTCGGCGTGAAGCAGATC
GCCACCGGCGTGTCTGATACAATCTGCGAGCCCTGTCCTGTGGGCTTCTTTAGCAACGTGTCCTCTG
CCTTTGAGAAGTGCCACCCTTGGACCAGCTGTGAGACAAAGGACCTGGTGGTGCAGCAGGCCGGCAC
CAATAAGACAGATGTGGTGTGCGGACCTCAGGACAGGCTGAGAGCCCTGGTGGTCATCCCAATCATC
TTCGGCATCCTGTTTGCCATCCTGCTGGTGCTGGTGTTCATCGAGCGGACCATGCCTAGAATCCCAA
CACTGAAGAACCTGGAGGACCTGGTGACCGAGTACCACGGCAATTTCAGCGCCTGGTCCGGCGTGTC
TAAGGGCCTGGCCGAGAGCCTGCAGCCTGATTATTCCGAGCGGCTGTGCCTGGTGTCCGAGATCCCA
CCTAAGGGAGGCGCCCTGGGAGAGGGCCCTGGCGCCTCTCCTTGCAACCAGCACAGCCCATACTGGG
CCCCACCCTGTTATACCCTGAAGCCCGAGACA

SEQ ID NO: 32 is the amino acid sequence of an exemplary CD40-IL2RGtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRVVISVGSM
GLIISLLCVYFWLERTMPRIPTLKNLEDLVTEYHGNFSAWSGVSKGLAESLQPDYSERLCLVSEIPP
KGGALGEGPGASPCNQHSPYWAPPCYTLKPET

SEQ ID NO: 33 is an exemplary codon-optimized nucleic acid sequence of a CD40-IL2RGtm chimera:

ATGGTGAGGCTGCCACTGCAGTGCGTGCTGTGGGGATGTCTGCTGACCGCAGTGCACCCAGAGCCAC
CAACAGCCTGTCGCGAGAAGCAGTACCTGATCAATTCTCAGTGCTGTAGCCTGTGCCAGCCCGGCCA
GAAGCTGGTGTCCGATTGTACCGAGTTCACCGAGACAGAGTGCCTGCCTTGTGGCGAGTCTGAGTTT
CTGGATACCTGGAACCGGGAGACACACTGCCACCAGCACAAGTATTGTGACCCCAATCTGGGCCTGA
GAGTGCAGCAGAAGGGCACCTCCGAGACCGACACAATCTGCACATGTGAGGAGGGATGGCACTGCAC
CAGCGAGGCATGCGAGTCCTGCGTGCTGCACCGCTCCTGTTCTCCCGGCTTCGGCGTGAAGCAGATC
GCCACCGGCGTGTCTGATACAATCTGCGAGCCCTGTCCTGTGGGCTTCTTTAGCAACGTGTCCTCTG
CCTTTGAGAAGTGCCACCCTTGGACCAGCTGTGAGACAAAGGACCTGGTGGTGCAGCAGGCCGGCAC
CAATAAGACAGATGTGGTGTGCGGACCTCAGGACAGGCTGAGAGTGGTCATCAGCGTGGGCAGCATG
GGCCTGATCATCTCTCTGCTGTGCGTGTACTTCTGGCTGGAGCGGACCATGCCTAGAATCCCAACAC
TGAAGAACCTGGAGGACCTGGTGACCGAGTACCACGGCAATTTCAGCGCCTGGTCCGGCGTGTCTAA
GGGCCTGGCCGAGAGCCTGCAGCCTGATTATTCCGAGCGGCTGTGCCTGGTGTCCGAGATCCCACCT
AAGGGAGGCGCCCTGGGAGAGGGCCCTGGCGCCTCTCCTTGCAACCAGCACAGCCCATACTGGGCCC
CACCCTGTTATACCCTGAAGCCCGAGACA

SEQ ID NO: 34 is the amino acid sequence of an exemplary CD40-IGF1Rtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRLIIALPVA
VLLIVGGLVIMLYVFHRKRNNSRLGNGVLYASVNPEYFSAADVYVPDEWEVAREKITMSRELGQGSF
GMVYEGVAKGVVKDEPETRVAIKTVNEAASMRERIEFLNEASVMKEFNCHHVVRLLGVVSQGQPTLV
IMELMTRGDLKSYLRSLRPEMENNPVLAPPSLSKMIQMAGEIADGMAYLNANKFVHRDLAARNCMVA
EDFTVKIGDFGMTRDIYETDYYRKGGKGLLPVRWMSPESLKDGVFTTYSDVWSFGVVLWEIATLAEQ
PYQGLSNEQVLRFVMEGGLLDKPDNCPDMLFELMRMCWQYNPKMRPSFLEIISSIKEEMEPGFREVS
FYYSEENKLPEPEELDLEPENMESVPLDPSASSSSLPLPDRHSGHKAENGPGPGVLVLRASFDERQP
YAHMNGGRKNERALPLPQSSTC

SEQ ID NO: 35 is an exemplary codon-optimized nucleic acid sequence of a CD40-IGF1Rtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGACTGATCATCGCCCTGCCAGTGGCC
GTGCTGCTGATCGTGGGAGGCCTGGTCATCATGCTGTACGTGTTCCACCGGAAGAGAAACAATAGCC
GGCTGGGAAACGGCGTGCTGTACGCCTCTGTGAATCCAGAGTACTTCAGCGCCGCCGACGTGTACGT
GCCCGATGAGTGGGAGGTGGCCAGAGAGAAGATCACCATGTCCAGAGAGCTGGGCCAGGGCTCTTTC
GGCATGGTGTACGAGGGCGTGGCCAAGGGCGTGGTGAAGGATGAGCCCGAGACCAGAGTGGCCATCA
AGACAGTGAACGAGGCCGCCAGCATGAGGGAGCGCATCGAGTTCCTGAACGAGGCCTCCGTGATGAA
GGAGTTTAATTGCCACCACGTGGTGAGGCTGCTGGGAGTGGTGTCCCAGGGACAGCCCACCCTGGTC
ATCATGGAGCTGATGACACGCGGCGACCTGAAGAGCTATCTGCGGTCCCTGAGACCTGAGATGGAGA
ACAATCCAGTGCTGGCCCCCCCTTCTCTGAGCAAGATGATCCAGATGGCCGGCGAGATCGCCGATGG
CATGGCCTACCTGAACGCCAATAAGTTCGTGCACCGGGACCTGGCCGCCAGAAACTGTATGGTGGCC
GAGGACTTCACCGTGAAGATCGGCGATTTTGGCATGACCAGGGACATCTATGAGACAGATTACTATA
GGAAGGGAGGCAAGGGACTGCTGCCCGTGAGGTGGATGTCCCCTGAGTCTCTGAAGGACGGCGTGTT
CACCACATATTCTGACGTGTGGAGCTTTGGCGTGGTGCTGTGGGAGATCGCCACACTGGCCGAGCAG
CCATACCAGGGCCTGTCCAATGAGCAGGTGCTGCGCTTTGTGATGGAGGGCGGCCTGCTGGACAAGC
CCGATAACTGCCCTGATATGCTGTTCGAGCTGATGCGGATGTGCTGGCAGTACAATCCAAAGATGAG
ACCCTCTTTTCTGGAGATCATCAGCTCCATCAAGGAGGAGATGGAGCCTGGCTTCCGGGAGGTGTCC
TTTTACTATTCTGAGGAGAACAAGCTGCCAGAGCCAGAGGAGCTGGACCTGGAGCCAGAGAATATGG
AGAGCGTGCCCCTGGACCCCTCCGCCTCTAGCTCCTCTCTGCCTCTGCCAGACAGACACTCTGGCCA
CAAGGCCGAGAATGGCCCCGGCCCTGGCGTGCTGGTGCTGAGGGCCAGCTTTGACGAGCGCCAGCCT
TACGCCCACATGAACGGCGGCAGGAAGAATGAGCGCGCCCTGCCACTGCCCCAGAGCAGCACCTGC

SEQ ID NO: 36 is the amino acid sequence of an exemplary CD40-EGFRtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRIATGMVGA
LLLLLVVAGIGLFMRRRHIVRKRTLRRLLQERELVEPLTPSGEAPNQALLRILKETEFKKIKVLGSG
AFGTVYKGLWIPEGEKVKIPVAIKELREATSPKANKEILDEAYVMASVDNPHVCRLLGICLTSTVQL
ITQLMPFGCLLDYVREHKDNIGSQYLLNWCVQIAKGMNYLEDRRLVHRDLAARNVLVKTPQHVKITD
FGLAKLLGAEEKEYHAEGGKVPIKWMALESILHRIYTHQSDVWSYGVTVWELMTFGSKPYDGIPASE
ISSILEKGERLPQPPICTIDVYMIMVKCWMIDADSRPKFRELIIEFSKMARDPQRYLVIQGDERMHL
PSPTDSNFYRALMDEEDMDDVVDADEYLIPQQGFFSSPSTSRTPLLSSLSATSNNSTVACIDRNGLQ
SCPIKEDSFLQRYSSDPTGALTEDSIDDTFLPVPEYINQSVPKRPAGSVQNPVYHNQPLNPAPSRDP
HYQDPHSTAVGNPEYLNTVQPTCVNSTFDSPAHWAQKGSHQISLDNPDYQQDFFPKEAKPNGIFKGS
TAENAEYLRVAPQSSEFIGA

SEQ ID NO: 37 is an exemplary codon-optimized nucleic acid sequence of a CD40-EGFRtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATCGCCACCGGCATGGTGGGAGCA
CTGCTGCTGCTGCTGGTTGTAGCCCTCGGCATTGGACTGTTCATGAGACGGAGACACATCGTGCGAA
AGAGAACACTGAGGCGGCTGCTGCAAGAGCGGGAACTGGTCGAGCCTCTGACACCTAGCGGCGAGGC
CCCTAATCAGGCCCTCCTGAGAATCCTGAAGGAAACCGAGTTTAAGAAGATCAAGGTGCTGGGATCT
GGCGCTTTTGGTACAGTGTATAAGGGCCTGTGGATCCCCGAGGGCGAGAAAGTGAAAATCCCCGTGG
CCATCAAAGAGCTGAGAGAGGCCACATCTCCTAAGGCCAACAAGGAAATCCTCGATGAGGCCTACGT
CATGGCCAGCGTTGACAACCCGCACGTGTGCAGACTGCTGGGCATTTGTCTGACTAGCACAGTGCAG
CTGATCACACAGCTGATGCCTTTCGGCTGTCTGCTGGACTACGTGCGGGAACACAAGGACAACATCG
GCAGCCAGTACCTGCTTAATTGGTGCGTGCAAATCGCCAAGGGCATGAACTACCTGGAGGATAGAAG
ACTGGTGCACAGAGATCTGGCTGCTAGAAACGTGCTGGTGAAGACCCCTCAGCACGTGAAGATCACA
GACTTCGGCCTGGCCAAGCTGCTGGGCGCCGAGGAAAAGGAGTATCACGCCGAAGGAGGCAAGGTGC
CTATCAAGTGGATGGCTCTGGAAAGCATCCTGCACCGAATCTACACCCACCAGAGCGACGTGTGGTC
CTACGGCGTGACCGTGTGGGAACTGATGACCTTCGGCAGCAAGCCTTACGACGGCATCCCTGCCTCC
GAGATCTCTTCTATCCTGGAAAAAGGCGAGCGGCTGCCTCAGCCTCCTATCTGCACCATCGACGTGT
ACATGATCATGGTCAAATGCTGGATGATCGATGCCGATTCCAGACCTAAGTTCAGAGAGCTGATTAT
CGAGTTTAGCAAGATGGCCAGAGACCCTCAGCGGTACCTGGTGATCCAGGGCGACGAGAGAATGCAC
CTGCCCAGTCCAACCGACAGCAACTTCTACCGGGCCCTGATGGACGAAGAGGACATGGATGATGTGG
TCGACGCCGACGAGTACCTGATCCCACAGCAGGGATTTTTCAGCAGCCCTAGCACCAGCAGAACCCC
TCTGCTCAGCAGCCTGAGCGCCACCTCCAACAATAGCACAGTGGCCTGCATTGACAGAAACGGCCTG
CAGTCCTGCCCCATCAAAGAGGACAGCTTCCTTCAGAGATACAGCTCTGATCCCACCGGCGCCCTGA
CCGAGGATTCTATCGACGACACCTTCCTGCCAGTTCCTGAGTACATCAACCAGTCTGTGCCAAAGCG
GCCCGCAGGCAGCGTGCAGAACCCCGTGTACCATAACCAGCCCCTGAACCCTGCTCCTAGCCGCGAC
CCTCACTACCAAGATCCTCACAGCACCGCCGTGGGCAATCCTGAATATCTGAACACCGTGCAGCCTA
CCTGTGTGAACAGCACATTCGACAGCCCCGCTCATTGGGCCCAGAAGGGCAGCCACCAGATCAGCCT
GGACAATCCTGATTACCAGCAGGACTTCTTCCCCAAGGAAGCTAAGCCCAACGGCATCTTCAAAGGA
AGCACCGCCGAGAACGCCGAGTACCTGAGAGTGGCCCCACAATCTTCTGAGTTCATCGGAGCC

SEQ ID NO: 38 is the amino acid sequence of an exemplary CD40-TrkAtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRLAVFACLF
LSTLLLVLNKCGRRNKFGINRPAVLAPEDGLAMSLHFMTLGGSSLSPTEGKGSGLQGHIIENPQYFS
DACVHHIKRRDIVLKWELGEGAFGKVFLAECHNLLPEQDKMLVAVKALKEASESARQDFQREAELLT
MLQHQHIVRFFGVCTEGRPLLMVFEYMRHGDLNRFLRSHGPDAKLLAGGEDVAPGPLGLGQLLAVAS
QVAAGMVYLAGLHFVHRDLATRNCLVGQGLVVKIGDFGMSRDIYSTDYYRVGGRTMLPIRWMPPESI
LYRKFTTESDVWSFGVVLWEIFTYGKQPWYQLSNTEAIDCITQGRELERPRACPPEVYAIMRGCWQR
EPQQRHSIKDVHARLQALAQAPPVYLDVLG

SEQ ID NO: 39 is an exemplary codon-optimized nucleic acid sequence of a CD40-TrkAtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGACTCGCCGTGTTCGCCTGTCTGTTC
CTTTCTACCCTGCTGCTCGTGCTGAACAAGTGCGGCAGGCGGAACAAATTCGGCATCAACAGACCCG
CCGTGCTGGCACCTGAAGATGGCCTGGCCATGAGCCTGCACTTTATGACACTGGGCGGATCTAGCCT
GTCTCCTACAGAAGGCAAGGGCAGCGGACTGCAGGGCCACATCATCGAGAATCCTCAATACTTCAGC
GACGCCTGTGTGCACCACATTAAGCGGAGAGACATCGTGCTGAAATGGGAACTGGGCGAAGGCGCCT
TCGGAAAGGTGTTCCTGGCCGAGTGCCACAACCTGCTGCCTGAGCAGGATAAGATGCTGGTGGCCGT
GAAAGCCCTGAAGGAAGCTAGCGAGTCCGCCAGACAGGACTTTCAGCGGGAAGCCGAACTGCTGACA
ATGCTGCAGCACCAGCACATCGTGCGGTTCTTTGGTGTTTGTACCGAAGGCAGACCTCTGCTGATGG
TCTTTGAGTACATGCGGCATGGCGATCTGAATCGGTTCCTGAGGAGCCACGGCCCTGATGCCAAGCT
GCTGGCTGGCGGAGAGGACGTGGCTCCCGGCCCCCTGGGCCTGGGACAACTCCTGGCTGTTGCCAGC
CAGGTGGCCGCTGGCATGGTGTACCTGGCCGGCCTGCACTTCGTGCATAGAGACCTGGCCACCAGAA
ACTGCCTGGTGGGCCAGGGCCTGGTCGTGAAGATCGGCGACTTCGGCATGAGCAGAGACATCTACAG
CACAGACTACTACAGAGTGGGCGGCCGCACCATGCTGCCAATCAGATGGATGCCTCCAGAGAGCATC
CTGTACCGGAAGTTCACCACCGAGTCTGATGTGTGGTCCTTCGGCGTGGTGCTGTGGGAGATCTTCA
CCTACGGCAAGCAGCCTTGGTATCAGCTGAGCAACACCGAGGCCATCGACTGCATCACCCAGGGCAG
AGAGCTGGAAAGACCGCGGGCCTGCCCCCCCGAGGTGTACGCCATCATGAGAGGCTGCTGGCAGAGA
GAGCCTCAGCAGAGACACAGCATCAAGGACGTGCACGCCAGATTGCAAGCTCTGGCCCAGGCCCCTC
CTGTGTATCTGGACGTGCTGGGA

SEO ID NO: 40 is the amino acid sequence of an exemplary CD40-TrkBtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRLSVYAVVV
IASVVGFCLLVMLFLLKLARHSKFGMKGPASVISNDDDSASPLHHISNGSNTPSSSEGGPDAVIIGM
TKIPVIENPQYFGITNSQLKPDTFVQHIKRHNIVLKRELGEGAFGKVFLAECYNLCPEQDKILVAVK
TLKDASDNARKDFHREAELLTNLQHEHIVKFYGVCVEGDPLIMVFEYMKHGDLNKFLRAHGPDAVLM
AEGNPPTELTQSQMLHIAQQIAAGMVYLASQHFVHRDLATRNCLVGENLLVKIGDFGMSRDVYSTDY
YRVGGHTMLPIRWMPPESIMYRKFTTESDVWSLGVVLWEIFTYGKQPWYQLSNNEVIECITQGRVLQ
RPRTCPQEVYELMLGCWQREPHMRKNIKGIHTLLQNLAKASPVYLDILG

SEQ ID NO: 41 is an exemplary codon-optimized nucleic acid sequence of a CD40-TrkBtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGACTCTCCGTGTACGCCGTAGTCGTG
ATCGCCTCCGTGGTTGGATTTTGCCTGCTGGTGATGCTGTTCCTGCTTAAGCTGGCTCGGCACAGCA
AGTTCGGAATGAAAGGCCCTGCTAGCGTGATTTCTAACGACGATGACAGCGCCTCTCCTCTGCACCA
CATCAGCAACGGCAGCAATACCCCTTCTAGCAGCGAGGGCGGCCCTGATGCTGTGATTATCGGTATG
ACCAAGATCCCTGTGATCGAAAATCCTCAGTACTTCGGCATCACAAACAGCCAACTGAAGCCCGACA
CCTTCGTGCAGCACATCAAGAGACACAATATCGTGCTGAAACGGGAACTGGGCGAGGGCGCCTTCGG
CAAAGTGTTCCTGGCCGAATGCTACAACCTGTGCCCTGAACAGGACAAGATCCTGGTGGCCGTGAAG
ACCCTGAAGGACGCCTCTGACAACGCCAGAAAGGACTTCCACCGGGAAGCCGAGCTGCTTACAAACC
TGCAGCACGAGCACATCGTGAAATTTTACGGCGTGTGCGTGGAAGGCGACCCCCTGATCATGGTGTT
CGAGTACATGAAGCACGGCGACCTGAACAAATTCCTGAGAGCCCACGGCCCTGATGCCGTTCTGATG
GCCGAGGGCAACCCTCCCACCGAGCTGACCCAGAGCCAGATGCTGCATATCGCCCAGCAGATCGCCG
CCGGCATGGTGTACCTGGCCAGCCAGCATTTCGTCCACCGCGACCTGGCCACCAGAAACTGCCTGGT
GGGAGAGAACCTGCTGGTGAAGATCGGCGATTTCGGCATGAGCAGAGATGTGTACAGCACCGACTAC
TACAGAGTGGGCGGCCACACCATGCTGCCTATCAGATGGATGCCTCCAGAAAGCATCATGTACCGGA
AGTTTACAACCGAGTCTGATGTGTGGTCCCTGGGAGTGGTCCTGTGGGAGATCTTCACATATGGAAA
GCAGCCTTGGTACCAGCTGAGCAACAATGAGGTGATTGAGTGTATCACACAGGGCAGAGTGCTGCAA
AGACCCAGAACCTGCCCCCAAGAGGTGTACGAGCTGATGCTGGGATGTTGGCAGCGGGAACCCCACA
TGAGGAAGAACATCAAGGGCATCCACACACTGCTGCAGAACCTCGCTAAGGCTTCTCCAGTGTATCT
GGACATCCTGGGC

SEQ ID NO: 42 is the amino acid sequence of an exemplary CD40-TrkCtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRFGVSIAVG
LAAFACVLLVVLFVMINKYGRRSKFGMKGPVAVISGEEDSASPLHHINHGITTPSSLDAGPDTVVIG
MTRIPVIENPQYFRQGHNCHKPDTYVQHIKRRDIVLKRELGEGAFGKVFLAECYNLSPTKDKMLVAV
KALKDPTLAARKDFQREAELLTNLQHEHIVKFYGVCGDGDPLIMVFEYMKHGDLNKFLRAHGPDAMI
LVDGQPRQAKGELGLSQMLHIASQIASGMVYLASQHFVHRDLATRNCLVGANLLVKIGDFGMSRDVY
STDYYRLFNPSGNDFCIWCEVGGHTMLPIRWMPPESIMYRKFTTESDVWSFGVILWEIFTYGKQPWE
QLSNTEVIECITQGRVLERPRVCPKEVYDVMLGCWQREPQQRLNIKEIYKILHALGKATPIYLDILG

SEQ ID NO: 43 is an exemplary codon-optimized nucleic acid sequence of a CD40-TrkCtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGATTCGGAGTCAGCATCGCCGTGGGC
CTGGCTGCTTTTGCCTGCGTGCTGCTCGTGGTCCTGTTCGTGATGATCAACAAATACGGCAGAAGGT
CCAAGTTCGGCATGAAAGGCCCAGTGGCCGTGATCAGCGGCGAGGAAGATAGCGCCTCTCCTCTGCA
TCACATTAACCACGGCATCACAACACCTTCCAGCCTGGACGCTGGCCCTGACACAGTGGTGATCGGC
ATGACAAGAATCCCTGTGATCGAGAACCCTCAGTACTTCCGGCAAGGCCACAACTGCCACAAGCCTG
ATACATACGTGCAGCATATCAAGCGGCGGGACATCGTGCTGAAGAGAGAGCTGGGAGAGGGCGCCTT
CGGCAAAGTGTTCTTGGCCGAGTGCTACAACCTGAGCCCTACCAAGGATAAGATGCTGGTCGCCGTG
AAGGCCCTGAAAGACCCCACCCTGGCCGCTAGAAAGGACTTCCAGCGGGAAGCCGAGCTGCTGACCA
ACCTGCAGCACGAGCACATCGTGAAATTCTACGGAGTGTGTGGCGACGGCGATCCCCTGATCATGGT
TTTTGAGTACATGAAGCACGGCGACCTGAACAAGTTCCTGAGAGCCCACGGCCCAGACGCCATGATC
CTGGTGGACGGCCAGCCTAGACAGGCTAAGGGCGAACTGGGCCTCTCCCAAATGCTGCACATCGCCA
GCCAGATCGCCAGCGGAATGGTGTATCTGGCCTCTCAGCACTTCGTGCACAGAGATCTGGCAACCAG
AAACTGCCTGGTGGGAGCCAACCTGCTGGTGAAGATCGGGGACTTCGGCATGAGCAGAGATGTGTAT
AGCACCGACTACTACCGGCTGTTTAATCCTAGCGGCAACGACTTCTGCATTTGGTGCGAGGTGGGAG
GCCACACAATGCTCCCCATCCGGTGGATGCCTCCTGAGAGCATCATGTACCGCAAGTTCACCACCGA
AAGCGACGTATGGTCTTTTGGCGTGATCCTGTGGGAAATCTTCACCTACGGCAAGCAGCCCTGGTTC
CAGCTGTCTAATACCGAGGTGATCGAGTGTATCACCCAGGGGAGAGTGCTGGAAAGACCCAGAGTGT
GCCCCAAGGAAGTGTACGACGTGATGCTGGGATGTTGGCAGAGAGAGCCCCAGCAGCGGCTGAATAT
CAAGGAAATTTACAAGATCCTGCACGCCCTGGGCAAGGCCACCCCTATCTACCTGGATATCCTGGGC

SEQ ID NO: 44 is the amino acid sequence of an exemplary CD40-MuSktm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRVIISIMSS
FAIFVLLTITTLYCCRRRKQWKNKKRESAAVTLTTLPSELLLDRLHPNPMYQRMPLLLNPKLLSLEY
PRNNIEYVRDIGEGAFGRVFQARAPGLLPYEPFTMVAVKMLKEEASADMQADFQREAALMAEFDNPN
IVKLLGVCAVGKPMCLLFEYMAYGDLNEFLRSMSPHTVCSLSHSDLSMRAQVSSPGPPPLSCAEQLC
IARQVAAGMAYLSERKFVHRDLATRNCLVGENMVVKIADFGLSRNIYSADYYKANENDAIPIRWMPP
ESIFYNRYTTESDVWAYGVVLWEIFSYGLQPYYGMAHEEVIYYVRDGNILSCPENCPVELYNLMRLC
WSKLPADRPSFTSIHRILERMCERAEGTVSV

SEQ ID NO: 45 is an exemplary codon-optimized nucleic acid sequence of a CD40-MuSktm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAGTCATCATCAGCATCATGTCTTCT
TTTGCCATCTTCGTGCTGCTCACCATCACAACACTGTACTGCTGCAGACGGAGAAAGCAGTGGAAGA
ACAAGAAAAGAGAATCTGCTGCTGTTACACTGACCACCCTGCCAAGCGAGCTGCTGCTTGATAGACT
GCATCCTAATCCCATGTACCAGAGAATGCCCCTGCTCCTCAACCCTAAGCTGCTGAGCCTGGAATAC
CCCAGAAACAACATCGAGTACGTGCGGGACATCGGCGAGGGCGCCTTCGGCAGAGTGTTCCAGGCCC
GGGCTCCTGGACTGCTGCCTTACGAGCCTTTCACAATGGTGGCCGTGAAGATGCTGAAGGAAGAGGC
CAGCGCCGATATGCAGGCTGACTTCCAAAGAGAGGCCGCCCTGATGGCCGAGTTCGACAACCCCAAC
ATTGTGAAACTGTTGGGCGTGTGCGCCGTGGGAAAACCTATGTGTCTGCTGTTCGAGTACATGGCCT
ACGGCGATCTGAATGAGTTCCTGAGGTCCATGAGCCCCCACACCGTGTGTTCTCTGAGCCACAGCGA
CCTGTCCATGAGAGCCCAGGTGTCCAGCCCTGGCCCTCCACCACTGTCCTGCGCCGAACAGCTGTGC
ATCGCTCGCCAGGTGGCCGCTGGCATGGCTTACCTGTCTGAACGGAAGTTCGTGCACAGGGATCTGG
CCACCAGAAATTGTCTGGTGGGCGAAAACATGGTGGTCAAGATCGCCGATTTCGGCCTGAGCAGAAA
CATCTACAGCGCCGACTACTACAAGGCCAACGAGAACGACGCCATCCCCATCCGGTGGATGCCTCCT
GAGAGCATCTTTTACAATAGATACACCACAGAGAGCGACGTGTGGGCCTATGGAGTGGTGCTGTGGG
AGATCTTTAGCTACGGCCTGCAGCCTTACTACGGAATGGCCCACGAGGAAGTGATTTATTATGTGAG
AGACGGCAACATCCTGAGCTGTCCTGAGAACTGCCCCGTGGAACTGTACAACCTGATGCGGCTGTGC
TGGAGCAAGCTGCCTGCCGACAGACCTTCTTTCACAAGCATCCACCGGATCCTGGAAAGAATGTGCG
AGCGGGCCGAGGGCACCGTGAGCGTG

SEQ ID NO: 46 is the amino acid sequence of an exemplary CD40-Mettm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRGLIAGVVS
ISTALLLLLGFFLWLKKRKQIKDLGSELVRYDARVHTPHLDRLVSARSVSPTTEMVSNESVDYRATF
PEDQFPNSSQNGSCRQVQYPLTDMSPILTSGDSDISSPLLQNTVHIDLSALNPELVQAVQHVVIGPS
SLIVHFNEVIGRGHFGCVYHGTLLDNDGKKIHCAVKSLNRITDIGEVSQFLTEGIIMKDFSHPNVLS
LLGICLRSEGSPLVVLPYMKHGDLRNFIRNFTHNPTVKDLIGFGLQVAKGMKYLASKKFVHRDLAAR
NCMLDEKFTVKVADFGLARDMYDKEYYSVHNKTGAKLPVKWMALESLQTQKFTTKSDVWSFGVLLWE
LMTRGAPPYPDVNTFDITVYLLQGRRLLQPEYCPDPLYEVMLKCWHPKAEMRPSFSELVSRISAIFS
TFIGEHYVHVNATYVNVKCVAPYPSLLSSEDNADDEVDTRPASFWETS

SEQ ID NO: 47 is an exemplary codon-optimized nucleic acid sequence of a CD40-Mettm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAGGCCTGATCGCCGGCGTGGTATCT
ATCAGCACAGCCCTGCTGCTCCTCCTGGGCTTCTTCCTGTGGCTGAAAAAGCGGAAGCAGATCAAGG
ACCTGGGAAGCGAGCTGGTCAGATATGACGCCAGAGTGCACACCCCTCACCTGGATAGACTGGTGTC
TGCCAGAAGCGTCTCTCCTACCACAGAGATGGTGTCCAACGAGTCCGTGGACTACCGGGCCACATTC
CCAGAAGATCAGTTTCCTAACAGCAGCCAGAACGGCTCTTGTAGACAGGTGCAATACCCTCTGACCG
ACATGAGCCCTATCCTGACAAGCGGCGACAGCGATATCAGCAGCCCCCTACTGCAGAACACCGTTCA
TATCGACCTGTCTGCCCTGAACCCCGAACTGGTGCAGGCCGTGCAGCACGTGGTGATCGGTCCAAGC
TCTCTTATTGTGCACTTTAACGAGGTCATCGGAAGAGGCCACTTCGGCTGCGTGTACCACGGCACCC
TGCTGGACAACGACGGCAAGAAGATCCACTGCGCCGTGAAAAGCCTGAATAGAATCACAGATATCGG
CGAGGTGTCCCAGTTCCTGACCGAGGGCATCATCATGAAAGACTTCAGCCACCCTAATGTGCTGAGC
CTGCTGGGCATTTGCCTTCGGAGCGAGGGCAGCCCTCTGGTCGTGCTGCCTTACATGAAGCACGGCG
ATCTGAGAAACTTCATCCGGAATGAAACCCACAATCCTACAGTGAAGGATCTGATCGGATTTGGCCT
GCAGGTTGCAAAGGGCATGAAATACCTGGCTTCTAAGAAATTCGTGCATAGAGATCTGGCCGCTAGA
AACTGCATGCTGGACGAGAAGTTTACCGTGAAGGTGGCCGATTTCGGACTGGCCCGGGACATGTACG
ACAAGGAATACTACAGCGTGCACAACAAGACCGGCGCTAAGCTGCCCGTGAAGTGGATGGCCTTGGA
ATCCCTGCAAACACAGAAATTCACCACTAAGTCCGATGTGTGGTCCTTCGGCGTGCTGCTGTGGGAG
CTGATGACCCGGGGCGCTCCTCCTTATCCAGACGTGAACACATTCGACATCACCGTGTACCTGCTGC
AGGGCAGGCGGCTGCTGCAGCCTGAGTACTGCCCCGACCCCCTGTACGAGGTGATGCTGAAGTGCTG
GCACCCCAAGGCCGAGATGAGACCTAGCTTCAGCGAACTGGTGAGTAGAATCAGCGCTATCTTCAGC
ACCTTCATCGGAGAACACTACGTGCACGTGAACGCCACCTACGTGAACGTGAAGTGTGTGGCCCCTT
ACCCCAGCCTGCTGAGCAGCGAGGATAACGCCGACGACGAGGTGGACACCAGACCCGCCAGCTTCTG
GGAAACCAGC

SEQ ID NO: 48 is the amino acid sequence of an exemplary CD40-Axltm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRYVLLGAVV
AAACVLILALFLVHRRKKETRYGEVFEPTVERGELVVRYRVRKSYSRRTTEATLNSLGISEELKEKI
RDVMVDRHKVALGKTLGEGEFGAVMEGQLNQDDSILKVAVKTMKIAICTRSELEDELSEAVCMKEFD
HPNVMRLIGVCFQGSERESFPAPVVILPFMKHGDLHSFLLYSRLGDQPVYLPTQMLVKFMADIASGM
EYLSTKRFIHRDLAARNCMLNENMSVCVADFGLSKKIYNGDYYRQGRIAKMPVKWIAIESLADRVYT
SKSDVWSFGVTMWEIATRGQTPYPGVENSEIYDYLRQGNRLKQPADCLDGLYALMSRCWELNPQDRP
SFTELREDLENTLKALPPAQEPDEILYVNMDEGGGYPEPPGAAGGADPPTQPDPKDSCSCLTAAEVH
PAGRYVLCPSTTPSPAQPADRGSPAAPGQEDGA

A

SEQ ID NO: 49 is an exemplary codon-optimized nucleic acid sequence of a CD40-Axltm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGATATGTGCTGCTCGGCGCCGTGGTG
GCCGCCGCTTGTGTGCTGATCCTGGCCCTGTTCCTGGTTCATAGACGGAAGAAGGAAACCCGGTACG
GCGAGGTGTTCGAGCCCACCGTGGAAAGAGGAGAACTGGTGGTCAGATACCGGGTCAGAAAGAGCTA
CAGCAGAAGGACCACCGAGGCCACACTGAACAGCCTGGGCATCAGCGAGGAACTTAAAGAAAAGCTG
AGAGATGTGATGGTGGACCGGCACAAGGTGGCCCTGGGCAAGACCCTGGGGGAAGGCGAGTTTGGCG
CAGTGATGGAAGGCCAACTGAACCAGGATGATAGCATCCTGAAGGTGGCCGTCAAGACCATGAAGAT
CGCTATCTGCACCCGCAGCGAGCTCGAGGACTTCCTGAGCGAGGCCGTTTGCATGAAAGAGTTCGAC
CACCCCAACGTGATGCGGCTGATCGGCGTGTGCTTCCAGGGCTCTGAAAGAGAGAGCTTCCCCGCCC
CTGTGGTGATCCTGCCTTTCATGAAGCACGGCGATCTGCACTCCTTTCTGCTGTACAGCAGACTGGG
CGACCAGCCTGTGTACCTGCCCACACAGATGCTGGTGAAGTTCATGGCCGACATCGCCAGCGGCATG
GAATACCTGAGCACCAAGCGGTTCATTCACCGGGACCTGGCCGCCAGAAACTGCATGCTGAATGAGA
ACATGAGCGTGTGCGTGGCCGATTTTGGCCTGTCTAAAAAGATCTACAACGGCGACTACTACAGACA
GGGCAGAATCGCCAAGATGCCTGTGAAGTGGATCGCCATCGAGTCTCTGGCTGATAGAGTGTACACC
AGCAAGAGCGACGTGTGGTCCTTCGGCGTGACCATGTGGGAGATTGCCACACGGGGCCAAACACCAT
ATCCCGGAGTGGAAAATAGCGAGATCTACGACTACCTGAGACAGGGAAATAGACTGAAACAGCCTGC
TGACTGCCTGGACGGCCTGTACGCCCTGATGAGCCGGTGCTGGGAACTGAACCCCCAGGACAGACCT
TCTTTCACCGAGCTGAGAGAAGATCTGGAAAACACACTGAAAGCCCTGCCACCAGCCCAGGAGCCTG
ACGAGATCCTGTATGTGAACATGGACGAGGGCGGAGGCTACCCGGAACCTCCAGGAGCCGCCGGCGG
CGCCGACCCTCCTACCCAGCCTGATCCTAAGGACTCCTGCTCTTGTCTGACAGCCGCCGAGGTGCAC
CCTGCCGGAAGATACGTGCTGTGTCCTTCCACAACCCCTAGCCCTGCTCAGCCCGCCGATCGGGGCA
GCCCCGCTGCTCCTGGCCAGGAGGACGGCGCC

SEQ ID NO: 50 is the amino acid sequence of an exemplary CD40-Tie2tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRIAILGSAG
MTCLTVLLAFLIILQLKRANVQRRMAQAFQNVREEPAVQFNSGTLALNRKVKNNPDPTIYPVLDWND
IKFQDVIGEGNFGQVLKARIKKDGLRMDAAIKRMKEYASKDDHRDFAGELEVLCKLGHHPNIINLLG
ACEHRGYLYLAIEYAPHGNLLDFLRKSRVLETDPAFAIANSTASTLSSQQLLHFAADVARGMDYLSQ
KQFIHRDLAARNILVGENYVAKIADFGLSRGQEVYVKKTMGRLPVRWMAIESLNYSVYTTNSDVWSY
GVLLWEIVSLGGTPYCGMTCAELYEKLPQGYRLEKPLNCDDEVYDLMRQCWREKPYERPSFAQILVS
LNRMLEERKTYVNTTLYEKFTYAGIDCSAEEAA

SEQ ID NO: 51 is an exemplary codon-optimized nucleic acid sequence of a CD40-Tie2tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATCGCCATCCTGGGCAGCGCTGGC
ATGACATGCCTGACCGTGCTCCTGGCTTTCCTGATCATCCTGCAGCTGAAGAGAGCCAATGTGCAGA
GAAGAATGGCCCAGGCCTTTCAGAACGTGCGGGAAGAGCCTGCCGTGCAGTTTAACAGCGGCACACT
GGCCCTGAACAGAAAGGTGAAAAACAACCCTGATCCCACCATCTACCCTGTGCTGGATTGGAACGAC
ATCAAGTTCCAGGACGTTATCGGCGAAGGCAACTTCGGCCAGGTGCTGAAGGCCAGAATCAAGAAGG
ACGGCCTGAGAATGGATGCCGCTATTAAGCGGATGAAGGAATACGCCTCTAAGGATGACCACAGAGA
CTTCGCCGGCGAGCTGGAAGTGCTTTGTAAACTGGGCCACCATCCTAATATCATCAACCTGCTCGGC
GCTTGTGAACACAGAGGCTATCTGTACCTGGCTATCGAGTACGCCCCTCACGGAAATCTGCTGGACT
TCCTGCGGAAAAGCAGAGTGCTTGAGACAGACCCCGCCTTCGCAATCGCCAACAGCACCGCCTCTAC
CCTGTCCAGCCAACAGCTGCTGCACTTCGCTGCCGACGTGGCTAGAGGCATGGACTACCTGAGCCAG
AAACAGTTCATCCACCGGGATCTGGCCGCCCGCAACATCCTGGTCGGAGAAAACTACGTCGCCAAAA
TCGCCGATTTCGGACTGAGCCGGGGACAGGAGGTCTACGTGAAGAAGACAATGGGCAGGCTGCCAGT
GCGGTGGATGGCCATTGAGAGCCTGAACTACTCCGTGTATACAACAAACAGCGATGTGTGGAGCTAC
GGCGTGCTGCTGTGGGAGATCGTGTCTCTGGGCGGGACCCCTTACTGCGGCATGACCTGCGCCGAGC
TGTATGAGAAGCTGCCCCAGGGCTACAGACTGGAAAAGCCCCTGAACTGCGACGACGAGGTGTACGA
CCTGATGCGGCAGTGCTGGAGAGAGAAGCCTTACGAGCGTCCATCTTTTGCCCAAATCCTGGTGTCC
CTGAATAGAATGCTGGAAGAAAGAAAGACCTACGTGAACACCACACTGTACGAGAAGTTCACCTACG
CCGGCATCGACTGCAGCGCCGAGGAGGCCGCC

SEQ ID NO: 52 is the amino acid sequence of an exemplary CD40-EphA1tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRIVAVIFGL
LLGAALLLGILVERSRRAQRQRQQRORDRATDVDREDKLWLKPYVDLQAYEDPAQGALDFTRELDPA
WLMVDTVIGEGEFGEVYRGTLRLPSQDCKTVAIKTLKDTSPGGQWWNFLREATIMGQFSHPHILHLE
GVVTKRKPIMIITEFMENGALDAFLREREDQLVPGQLVAMLOGIASGMNYLSNHNYVHRDLAARNIL
VNQNLCCKVSDFGLTRLLDDFDGTYETQGGKIPIRWTAPEAIAHRIFTTASDVWSFGIVMWEVLSFG
DKPYGEMSNQEVMKSIEDGYRLPPPVDCPAPLYELMKNCWAYDRARRPHFQKLQAHLEQLLANPHSL
RTIANFDPRMTLRLPSLSGSDGIPYRTVSEWLESIRMKRYILHFHSAGLDTMECVLELTAEDLTQMG
ITLPGHQKRILCSIQGFKD

SEQ ID NO: 53 is an exemplary codon-optimized nucleic acid sequence of a CD40-EphA1tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATTGTGGCCGTGATCTTTGGACTG
CTGCTGGGAGCTGCTCTGCTGCTGGGCATCCTGGTGTTCAGATCTAGAAGAGCCCAGAGGCAGCGGC
AGCAGAGGCAGCGGGACAGAGCAACCGACGTGGACCGGGAAGATAAGCTGTGGCTGAAACCTTACGT
GGACCTGCAAGCTTATGAGGATCCAGCTCAGGGCGCCCTGGATTTCACCAGAGAACTGGATCCCGCC
TGGCTGATGGTGGACACAGTTATCGGCGAGGGAGAGTTCGGCGAGGTGTACAGAGGCACCCTGCGGC
TGCCCAGCCAGGACTGCAAGACAGTGGCCATCAAGACCCTGAAGGACACCAGCCCTGGCGGACAGTG
GTGGAATTTCCTGCGGGAAGCCACCATCATGGGCCAATTTAGCCACCCCCACATCCTGCACCTGGAA
GGCGTGGTGACCAAGAGAAAGCCTATCATGATCATCACCGAGTTCATGGAAAACGGCGCCCTGGACG
CCTTCCTGAGAGAGAGAGAGGACCAGCTGGTGCCAGGACAGCTGGTTGCTATGCTGCAAGGTATCGC
CTCCGGCATGAACTACCTGAGCAATCACAACTACGTGCACAGAGATCTGGCCGCCAGAAACATCCTG
GTCAACCAGAACCTGTGCTGTAAAGTGAGCGACTTCGGACTGACCAGATTGCTCGACGACTTCGATG
GCACATACGAAACCCAGGGCGGCAAGATCCCCATCCGGTGGACAGCCCCTGAGGCCATCGCCCACCG
GATCTTTACAACCGCCAGCGACGTGTGGTCCTTCGGCATCGTGATGTGGGAGGTGCTGTCTTTCGGC
GATAAGCCCTACGGCGAGATGAGCAACCAGGAGGTCATGAAGAGCATCGAGGATGGCTACCGGCTTC
CTCCACCTGTGGATTGCCCTGCTCCTCTGTACGAGCTGATGAAAAACTGCTGGGCCTACGACAGAGC
CAGACGGCCTCACTTTCAGAAACTGCAGGCCCATCTGGAACAGCTGCTGGCCAATCCTCATTCTCTG
CGTACCATCGCCAACTTCGACCCCAGAATGACCCTGAGACTGCCTAGCCTGAGCGGCTCTGACGGCA
TCCCTTATAGAACAGTGTCCGAGTGGTTGGAAAGCATCAGAATGAAGCGGTACATCCTGCACTTCCA
CAGCGCCGGCCTGGATACAATGGAGTGCGTGCTGGAGCTGACAGCCGAGGACCTGACACAAATGGGC
ATTACCCTGCCTGGCCACCAGAAGCGGATCCTGTGTAGCATCCAGGGTTTCAAGGAC

SEQ ID NO: 54 is the amino acid sequence of an exemplary CD40-EphA3tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRVVMIAISA
AVAIILLTVVIYVLIGRFCGYKSKHGADEKRLHFGNGHLKLPGLRTYVDPHTYEDPTQAVHEFAKEL
DATNISIDKVVGAGEFGEVCSGRLKLPSKKEISVAIKTLKVGYTEKQRRDFLGEASIMGQFDHPNII
RLEGVVTKSKPVMIVTEYMENGSLDSFLRKHDAQFTVIQLVGMLRGIASGMKYLSDMGYVHRDLAAR
NILINSNLVCKVSDFGLSRVLEDDPEAAYTTRGGKIPIRWTSPEAIAYRKFTSASDVWSYGIVLWEV
MSYGERPYWEMSNQDVIKAVDEGYRLPPPMDCPAALYQLMLDCWQKDRNNRPKFEQIVSILDKLIRN
PGSLKIITSAAARPSNLLLDQSNVDITTFRTTGDWLNGVWTAHCKEIFTGVEYSSCDTIAKISTDDM
KKVGVTVVGPQKKIISSIKALETQSKNGPVPV

SEQ ID NO: 55 is an exemplary codon-optimized nucleic acid sequence of a CD40-EphA3tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAGTGGTGATGATCGCCATCTCCGCC
GCCGTGGCCATCATTCTGCTGACCGTGGTTATCTACGTGCTTATCGGCAGATTCTGCGGCTATAAGA
GCAAGCACGGCGCCGACGAGAAGCGGCTGCACTTCGGCAACGGCCACCTGAAGCTGCCCGGCCTGAG
GACCTACGTGGATCCCCACACCTACGAAGACCCTACACAGGCCGTGCACGAGTTCGCCAAGGAACTG
GACGCCACAAACATCAGCATCGACAAGGTGGTGGGAGCTGGCGAGTTTGGCGAAGTGTGCAGCGGAA
GACTGAAGCTGCCTAGCAAGAAGGAAATCAGCGTGGCCATTAAGACCCTGAAGGTGGGATATACAGA
GAAGCAACGTAGAGATTTTCTGGGCGAGGCCTCCATCATGGGACAGTTCGACCACCCTAACATCATC
CGGCTGGAAGGCGTCGTGACCAAGTCTAAGCCCGTGATGATCGTGACCGAGTACATGGAAAACGGCA
GCCTGGACAGCTTCCTGAGAAAACATGACGCCCAGTTCACCGTGATCCAGCTGGTGGGAATGCTGCG
GGGAATCGCCTCTGGCATGAAATACCTGTCTGACATGGGCTACGTGCACAGAGACCTGGCGGCTAGA
AACATCCTGATTAACAGCAACCTGGTCTGCAAGGTGTCCGACTTCGGCCTGAGCAGAGTGCTGGAAG
ATGATCCTGAAGCTGCTTACACCACCAGAGGCGGCAAGATCCCCATCCGGTGGACCTCCCCTGAGGC
CATCGCCTACAGAAAGTTCACCAGCGCTTCTGATGTGTGGTCCTACGGAATCGTGCTGTGGGAGGTG
ATGTCCTACGGCGAGCGGCCTTACTGGGAGATGAGCAACCAGGACGTGATCAAGGCCGTTGACGAGG
GCTACCGGCTGCCTCCACCTATGGACTGTCCTGCCGCCCTGTACCAGCTGATGCTGGACTGCTGGCA
GAAAGACAGAAACAACCGCCCCAAGTTCGAGCAGATCGTCAGCATCCTCGATAAGCTGATCAGAAAT
CCTGGCAGCCTGAAAATCATCACCAGCGCCGCCGCCAGACCCTCTAATCTGCTGCTGGATCAGAGCA
ATGTGGACATCACAACTTTTAGAACCACAGGAGATTGGCTGAACGGCGTTTGGACAGCCCACTGTAA
AGAGATCTTCACAGGCGTGGAATACAGCTCATGCGACACCATCGCTAAGATCAGCACCGATGACATG
AAAAAGGTGGGCGTGACAGTGGTGGGCCCACAGAAGAAAATTATCAGCAGCATCAAGGCCCTCGAGA
CACAATCTAAGAACGGCCCTGTGCCTGTG

SEQ ID NO: 56 is the amino acid sequence of an exemplary CD40-EphA7tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRIIIAVVAV
AGTIILVFMVFGFIIGRRHCGYSKADQEGDEELYFHFKFPGTKTYIDPETYEDPNRAVHQFAKELDA
SCIKIERVIGAGEFGEVCSGRLKLPGKRDVAVAIKTLKVGYTEKQRRDFLCEASIMGQFDHPNVVHL
EGVVTRGKPVMIVIEFMENGALDAFLRKHDGQFTVIQLVGMLRGIAAGMRYLADMGYVHRDLAARNI
LVNSNLVCKVSDFGLSRVIEDDPEAVYTTTGGKIPVRWTAPEAIQYRKFTSASDVWSYGIVMWEVMS
YGERPYWDMSNQDVIKAIEEGYRLPAPMDCPAGLHOLMLDCWQKERAERPKFEQIVGILDKMIRNPN
SLKTPLGTCSRPISPLLDQNTPDFTTFCSVGEWLQAIKMERYKDNFTAAGYNSLESVARMTIEDVMS
LGITLVGHQKKIMSSIQTMRAQMLHLHGTGIQV

SEQ ID NO: 57 is an exemplary codon-optimized nucleic acid sequence of a CD40-EphA7tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATTATCATCGCCGTGGTGGCCGTG
GCCGGCACAATCATCCTGGTTTTCATGGTGTTCGGCTTCATCATTGGCAGACGGCACTGCGGCTACA
GCAAGGCCGATCAGGAGGGAGATGAGGAACTGTACTTCCACTTCAAGTTTCCAGGCACCAAGACCTA
CATCGACCCTGAGACATACGAGGACCCCAATAGAGCCGTGCACCAGTTTGCCAAGGAACTGGATGCC
AGCTGCATCAAGATCGAGCGGGTGATCGGCGCTGGAGAGTTCGGCGAGGTGTGTAGCGGCAGGCTGA
AGCTGCCTGGCAAAAGAGATGTGGCTGTGGCCATTAAGACCCTGAAAGTGGGATATACAGAGAAGCA
GAGAAGAGACTTTCTGTGCGAGGCATCTATCATGGGCCAGTTCGACCACCCTAATGTGGTGCACCTG
GAAGGCGTGGTCACCAGAGGCAAGCCTGTGATGATCGTAATTGAGTTCATGGAAAACGGCGCCCTGG
ACGCCTTCCTGAGAAAGCACGACGGCCAGTTCACCGTGATCCAGCTGGTCGGGATGCTGAGAGGAAT
CGCCGCTGGCATGCGGTACCTGGCCGACATGGGCTACGTGCACAGAGATCTGGCTGCTAGAAACATC
CTGGTGAACTCTAACTTGGTGTGCAAGGTGTCCGACTTTGGACTGAGCAGAGTGATCGAGGACGACC
CTGAGGCCGTGTACACCACCACCGGTGGAAAAATCCCCGTCAGGTGGACCGCCCCTGAAGCTATCCA
GTACAGAAAGITCACGAGCGCCTCTGACGTTTGGTCCTATGGCATCGTGATGTGGGAGGTGATGTCC
TACGGCGAGCGGCCCTACTGGGACATGAGCAACCAGGATGTGATCAAGGCCATCGAGGAAGGCTACC
GGCTGCCCGCTCCAATGGACTGTCCTGCCGGACTGCACCAACTGATGCTGGATTGCTGGCAGAAGGA
ACGGGCCGAGAGACCCAAGTTCGAGCAGATCGTGGGCATCCTGGACAAGATGATCCGGAACCCTAAT
AGCCTGAAGACACCTCTGGGCACCTGTAGCCGGCCAATCAGCCCTCTGCTGGACCAGAACACCCCTG
ACTTCACCACATTCTGCAGCGTGGGCGAATGGCTCCAGGCCATCAAAATGGAAAGATACAAGGACAA
CTTCACAGCCGCCGGCTACAACAGCCTGGAAAGCGTGGCCCGCATGACCATCGAGGATGTGATGAGC
CTGGGCATCACCCTGGTGGGCCACCAGAAAAAGATCATGTCTAGCATCCAAACAATGAGAGCCCAGA
TGCTTCATCTGCACGGCACCGGCATCCAAGTG

SEQ ID NO: 58 is the amino acid sequence of an exemplary CD40-EphA8tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRIVWICLTL
ITGLVVLLLLLICKKRHCGYSKAFQDSDEEKMHYQNGQAPPPVFLPLHHPPGKLPEPQFYAEPHTYE
EPGRAGRSFTREIEASRIHIEKIIGSGDSGEVCYGRLRVPGQRDVPVAIKALKAGYTERQRRDELSE
ASIMGQFDHPNIIRLEGVVTRGRLAMIVTEYMENGSLDTFLRTHDGQFTIMQLVGMLRGVGAGMRYL
SDLGYVHRDLAARNVLVDSNLVCKVSDFGLSRVLEDDPDAAYTTTGGKIPIRWTAPEAIAFRTFSSA
SDVWSFGVVMWEVLAYGERPYWNMTNRDVISSVEEGYRLPAPMGCPHALHQLMLDCWHKDRAQRPRF
SQIVSVLDALIRSPESLRATATVSRCPPPAFVRSCFDLRGGSGGGGGLTVGDWLDSIRMGRYRDHFA
AGGYSSLGMVLRMNAQDVRALGITLMGHQKKILGSIQTMRAQLTSTQGPRRHL

SEQ ID NO: 59 is an exemplary codon-optimized nucleic acid sequence of a CD40-EphA8tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATCGTGTGGATCTGTCTGACCCTG
ATCACAGGCCTGGTGGTGCTGCTGCTGCTGCTTATTTGCAAGAAGCGGCACTGCGGCTACAGCAAGG
CCTTTCAAGACTCTGACGAGGAAAAGATGCACTACCAGAACGGCCAGGCCCCTCCCCCTGTTTTCCT
GCCACTGCACCACCCTCCTGGGAAACTGCCCGAGCCCCAGTTCTACGCCGAACCTCACACCTACGAG
GAGCCCGGCAGAGCCGGCAGAAGCTTCACCCGGGAAATCGAGGCCAGCAGAATCCACATCGAGAAGA
TCATCGGCTCTGGTGATTCTGGCGAGGTGTGCTACGGCCGGCTGCGGGTGCCAGGCCAAAGAGACGT
GCCTGTGGCCATCAAGGCCCTGAAGGCCGGATATACCGAGCGGCAGAGACGGGACTTCCTGAGCGAG
GCCAGCATCATGGGCCAGTTTGACCACCCCAATATCATCAGACTGGAAGGCGTGGTGACCAGAGGAA
GACTGGCCATGATCGTGACAGAGTACATGGAAAACGGCAGCCTGGACACCTTCCTGCGGACCCACGA
CGGCCAGTTCACAATCATGCAGCTGGTGGGAATGCTGAGAGGCGTTGGAGCCGGCATGCGGTACCTG
AGCGATCTGGGCTACGTGCACAGAGATCTGGCTGCTAGAAACGTGCTGGTGGACAGCAACCTGGTCT
GTAAAGTGTCCGACTTCGGCCTGTCCAGAGTGCTCGAGGACGACCCCGACGCCGCTTACACCACAAC
AGGCGGCAAGATCCCCATCAGATGGACCGCCCCTGAGGCTATCGCCTTCAGAACCTTTAGCAGCGCC
AGCGATGTGTGGTCCTTCGGAGTGGTCATGTGGGAGGTTCTGGCCTACGGCGAACGACCTTACTGGA
ATATGACCAACAGAGACGTGATCAGCTCTGTGGAAGAAGGCTATAGACTGCCTGCTCCTATGGGCTG
TCCTCACGCCCTCCATCAGCTGATGCTGGACTGCTGGCACAAGGACAGAGCCCAGCGGCCTAGATTC
AGCCAGATCGTGAGTGTGTTGGATGCACTGATCCGGAGCCCTGAGAGCCTGAGAGCTACAGCCACAG
TGTCCCGGTGCCCTCCACCTGCCTTCGTCAGAAGCTGCTTCGATCTGAGAGGAGGCAGCGGCGGCGG
CGGCGGACTGACAGTGGGCGACTGGCTGGATTCTATCAGAATGGGCCGCTACAGAGATCACTTCGCC
GCCGGAGGCTACAGCAGCCTGGGGATGGTGCTGAGGATGAACGCCCAAGATGTGCGGGCCCTGGGCA
TCACCCTGATGGGACACCAGAAGAAAATTCTGGGCTCTATCCAGACCATGCGGGCCCAGCTGACCAG
CACCCAGGGCCCTAGAAGACATCTG

SEQ ID NO: 60 is the amino acid sequence of an exemplary CD40-EphB1tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRLIAGSAAA
GVVFVVSLVAISIVCSRKRAYSKEAVYSDKLQHYSTGRGSPGMKIYIDPFTYEDPNEAVREFAKEID
VSFVKIEEVIGAGEFGEVYKGRLKLPGKREIYVAIKTLKAGYSEKQRRDFLSEASIMGQFDHPNIIR
LEGVVTKSRPVMIITEFMENGALDSFLRQNDGQFTVIQLVGMLRGIAAGMKYLAEMNYVHRDLAARN
ILVNSNLVCKVSDFGLSRYLQDDTSDPTYTSSLGGKIPVRWTAPEAIAYRKFTSASDVWSYGIVMWE
VMSFGERPYWDMSNQDVINAIEQDYRLPPPMDCPAALHQLMLDCWQKDRNSRPRFAEIVNTLDKMIR
NPASLKTVATITAVPSQPLLDRSIPDFTAFTTVDDWLSAIKMVQYRDSFLTAGFTSLQLVTQMTSED
LLRIGITLAGHQKKILNSIHSMRVQISQSPTAMA

SEQ ID NO: 61 is an exemplary codon-optimized nucleic acid sequence of a CD40-EphBItm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGACTGATCGCCGGAAGCGCCGCCGCT
GGAGTGGTCTTTGTGGTGTCCCTGGTGGCCATCAGCATCGTGTGCAGCAGAAAGCGGGCCTATAGCA
AGGAGGCCGTGTACAGCGACAAGCTTCAACACTACAGCACCGGCAGAGGCTCCCCTGGCATGAAGAT
CTACATCGACCCTTTCACCTACGAGGATCCTAACGAGGCCGTGCGGGAGTTCGCCAAGGAAATCGAC
GTGTCCTTCGTGAAGATCGAAGAAGTGATTGGCGCAGGCGAGTTCGGCGAGGTTTATAAGGGCAGAC
TGAAACTGCCAGGCAAAAGAGAGATCTACGTGGCCATCAAGACCCTGAAGGCCGGCTACTCCGAAAA
GCAGCGGAGAGACTTCCTGAGTGAGGCCTCTATCATGGGCCAATTTGACCACCCCAACATCATTAGA
CTGGAAGGCGTGGTGACCAAAAGCAGACCCGTGATGATCATCACCGAGTTCATGGAAAACGGCGCCC
TGGATAGCTTCCTGCGACAGAACGACGGCCAGTTTACAGTGATCCAGCTGGTGGGCATGCTGCGGGG
CATCGCTGCCGGAATGAAGTACCTGGCTGAAATGAACTACGTGCACAGAGATCTGGCCGCCAGAAAC
ATCCTGGTGAATAGCAATCTGGTGTGCAAGGTGAGCGATTTTGGACTGAGCCGGTACCTGCAGGATG
ACACCTCTGATCCCACCTACACCAGCAGCCTGGGCGGCAAGATCCCCGTCAGATGGACCGCCCCTGA
GGCTATTGCCTACAGGAAGTTCACCAGCGCCTCTGACGTCTGGTCCTACGGCATCGTGATGTGGGAG
GTGATGAGCTTCGGAGAGCGGCCTTACTGGGACATGAGCAACCAGGACGTGATCAACGCCATCGAGC
AGGACTACAGACTCCCTCCACCTATGGACTGCCCTGCCGCTCTGCACCAGCTGATGCTGGACTGTTG
GCAGAAGGACAGAAACAGTCGCCCCCGGTTCGCCGAGATCGTGAACACCCTGGACAAGATGATCAGA
AATCCTGCTTCTCTGAAAACCGTTGCTACAATCACCGCCGTGCCCAGCCAGCCTCTGCTGGATAGAA
GCATCCCTGATTTCACCGCCTTCACAACAGTGGACGACTGGCTGTCTGCCATCAAGATGGTGCAGTA
CCGGGACAGCTTCCTGACCGCCGGCTTCACAAGCCTGCAACTGGTCACACAGATGACCTCCGAAGAT
CTGCTGAGAATCGGGATCACACTGGCCGGCCACCAGAAAAAGATCCTGAACAGCATCCATTCTATGA
GAGTGCAGATCTCTCAGAGCCCTACAGCTATGGCC

SEQ ID NO: 62 is the amino acid sequence of an exemplary CD40-EphB2tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRIIGSSAAG
LVFLIAVVVIAIVCNRRGFERADSEYTDKLQHYTSGHMTPGMKIYIDPFTYEDPNEAVREFAKEIDI
SCVKIEQVIGAGEFGEVCSGHLKLPGKREIFVAIKTLKSGYTEKQRRDFLSEASIMGQFDHPNVIHL
EGVVTKSTPVMIITEFMENGSLDSFLRQNDGQFTVIQLVGMLRGIAAGMKYLADMNYVHRDLAARNI
LVNSNLVCKVSDFGLSRFLEDDTSDPTYTSALGGKIPIRWTAPEAIQYRKFTSASDVWSYGIVMWEV
MSYGERPYWDMTNQDVINAIEQDYRLPPPMDCPSALHQLMLDCWQKDRNHRPKFGQIVNTLDKMIRN
PNSLKAMAPLSSGINLPLLDRTIPDYTSFNTVDEWLEAIKMGQYKESFANAGFTSFDVVSQMMMEDI
LRVGVTLAGHQKKILNSIQVMRAQMNQIQSVEGQPLARRPRATGRTKRCQPRDVTKKTCNSNDGKKK
GMGKKKTDPGRGREIQGIFFKEDSHKESNDCSCGG

SEQ ID NO: 63 is an exemplary codon-optimized nucleic acid sequence of a CD40-EphB2tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATCATCGGCAGCAGCGCAGCCGGC
CTGGTGTTCCTGATCGCCGTGGTGGTGATCGCCATCGTGTGCAACAGAAGGGGCTTCGAGCGGGCCG
ACAGCGAGTACACCGATAAGCTGCAACACTACACGTCCGGCCACATGACCCCAGGCATGAAGATCTA
CATCGACCCCTTCACCTACGAGGACCCTAACGAGGCTGTCAGAGAGTTCGCCAAGGAAATCGACATC
AGCTGCGTGAAGATCGAGCAAGTGATTGGAGCTGGCGAGTTCGGCGAAGTGTGTAGCGGCCACCTGA
AGCTGCCCGGCAAAAGAGAGATTTTTGTGGCCATCAAGACACTGAAGTCCGGTTATACAGAAAAGCA
GAGACGGGACTTCCTGAGCGAGGCCAGCATCATGGGCCAGTTTGATCACCCCAATGTGATCCACCTC
GAGGGTGTTGTGACTAAGAGCACCCCTGTGATGATCATCACCGAGTTCATGGAAAACGGCTCTCTGG
ATAGCTTCCTTAGACAGAACGACGGCCAATTTACAGTGATCCAGCTGGTGGGCATGCTGAGGGGCAT
CGCCGCTGGAATGAAGTACCTGGCTGATATGAACTACGTGCACAGAGATCTGGCCGCTAGAAACATC
CTGGTGAACAGCAATCTGGTCTGCAAGGTGTCCGATTTCGGCCTGAGCAGATTCCTGGAGGACGACA
CCTCTGATCCTACCTACACATCTGCCCTGGGCGGAAAGATCCCTATCCGGTGGACCGCCCCTGAGGC
CATTCAGTACCGGAAGTTCACATCCGCCTCTGACGTGTGGAGCTACGGCATCGTGATGTGGGAGGTG
ATGAGCTACGGAGAGAGACCCTACTGGGATATGACAAACCAGGACGTGATCAACGCCATCGAGCAGG
ACTACAGACTGCCTCCACCTATGGACTGCCCCAGCGCCCTGCACCAGCTGATGCTGGACTGCTGGCA
GAAGGACAGAAACCACCGGCCCAAGTTCGGGCAGATCGTCAACACCCTGGACAAGATGATCAGAAAT
CCTAACAGCCTGAAAGCTATGGCCCCTCTGTCTAGCGGCATCAACCTGCCACTGCTGGACCGCACCA
TCCCTGATTACACCAGCTTTAACACCGTGGACGAGTGGCTGGAAGCCATCAAGATGGGCCAGTATAA
GGAATCTTTCGCCAACGCCGGATTTACCAGCTTCGACGTGGTTAGCCAGATGATGATGGAAGATATC
CTGCGGGTGGGAGTGACCCTGGCTGGACATCAGAAAAAAATCCTGAACTCCATTCAGGTGATGAGAG
CCCAGATGAACCAGATCCAGTCTGTGGAAGGCCAGCCTCTAGCCAGACGGCCTAGAGCCACCGGCCG
GACCAAGAGATGCCAGCCCAGAGACGTGACAAAGAAGACCTGTAATAGCAATGATGGCAAGAAGAAA
GGCATGGGAAAGAAAAAAACAGACCCTGGCCGGGGCAGAGAGATCCAAGGCATCTTCTTCAAGGAAG
ATAGCCACAAGGAGAGCAACGACTGTAGCTGCGGCGGC

SEQ ID NO: 64 is the amino acid sequence of an exemplary CD40-EphB3tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRIVGSATAG
LVFVVAVVVIAIVCLRKQRHGSDSEYTEKLQQYIAPGMKVYIDPFTYEDPNEAVREFAKEIDVSCVK
IEEVIGAGEFGEVCRGRLKQPGRREVFVAIKTLKVGYTERQRRDFLSEASIMGQFDHPNIIRLEGVV
TKSRPVMILTEFMENCALDSFLRLNDGQFTVIQLVGMLRGIAAGMKYLSEMNYVHRDLAARNILVNS
NLVCKVSDFGLSRFLEDDPSDPTYTSSLGGKIPIRWTAPEAIAYRKFTSASDVWSYGIVMWEVMSYG
ERPYWDMSNQDVINAVEQDYRLPPPMDCPTALHQLMLDCWVRDRNLRPKFSQIVNTLDKLIRNAASL
KVIASAQSGMSQPLLDRTVPDYTTFTTVGDWLDAIKMGRYKESFVSAGFASFDLVAQMTAEDLLRIG
VTLAGHQKKILSSIQDMRLQMNQTLPVQV

SEQ ID NO: 65 is an exemplary codon-optimized nucleic acid sequence of a CD40-EphB3tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATTGTGGGGAGCGCTACAGCTGGA
CTGGTGTTCGTGGTGGCCGTGGTGGTGATCGCTATCGTGTGCCTGCGGAAGCAGCGGCACGGCAGCG
ATAGCGAGTACACCGAGAAGCTGCAGCAGTACATCGCCCCTGGAATGAAAGTGTACATCGACCCCTT
CACCTACGAGGATCCTAACGAGGCCGTTAGAGAGTTCGCCAAGGAAATCGACGTGTCCTGCGTGAAG
ATCGAGGAAGTGATCGGCGCCGGCGAATTTGGCGAGGTTTGTAGAGGCAGACTGAAACAGCCTGGCA
GGAGGGAGGTGTTCGTGGCCATCAAGACTCTGAAGGTCGGCTACACCGAAAGACAGAGACGGGACTT
CCTGAGCGAGGCCTCCATCATGGGCCAGTTTGACCACCCTAATATCATCAGACTGGAAGGCGTGGTC
ACCAAGAGCCGGCCCGTGATGATCCTGACCGAGTTCATGGAAAACTGCGCCCTGGACAGCTTTCTGC
GGCTCAACGACGGCCAGTTCACAGTGATCCAGCTCGTGGGAATGCTGCGCGGAATCGCCGCCGGCAT
GAAATACCTGAGCGAGATGAACTACGTGCATAGAGATCTGGCTGCCAGAAACATCCTGGTTAACAGC
AACCTGGTGTGTAAAGTGTCCGATTTCGGCCTGAGCAGATTCCTGGAAGATGACCCTTCTGATCCTA
CCTACACCAGCAGCCTCGGCGGCAAGATCCCCATCCGGTGGACCGCCCCTGAGGCCATCGCTTATAG
AAAGTTCACAAGCGCCAGCGACGTGTGGTCCTACGGCATCGTGATGTGGGAGGTGATGTCTTATGGC
GAGAGACCCTACTGGGACATGAGCAATCAAGATGTGATCAACGCCGTCGAGCAGGACTACAGACTTC
CACCTCCAATGGACTGCCCCACCGCCCTGCACCAGCTGATGCTGGATTGCTGGGTGAGAGACAGAAA
TCTGCGGCCTAAGTTCAGCCAAATCGTGAACACCCTGGATAAGCTGATTAGAAACGCCGCATCTCTG
AAGGTGATCGCCTCTGCTCAAAGCGGCATGAGCCAGCCCCTGCTGGACCGGACCGTGCCTGACTACA
CAACATTCACCACAGTGGGCGACTGGCTGGACGCCATCAAGATGGGCAGATACAAGGAAAGCTTCGT
GTCTGCCGGATTTGCCTCCTTCGACCTGGTCGCCCAGATGACAGCCGAGGACCTGCTGAGAATCGGA
GTGACCCTGGCCGGCCACCAGAAAAAGATCCTGTCTAGCATCCAGGACATGAGACTGCAGATGAACC
AGACCCTGCCTGTGCAGGTG

SEQ ID NO: 66 is the amino acid sequence of an exemplary CD40-EphB6tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRLVIGSILG
ALAFLLLAAITVLAVVFQRKRRGTGYTEQLQQYSSPGLGVKYYIDPSTYEDPCQAIRELAREVDPAY
IKIEEVIGTGSFGEVRQGRLQPRGRREQTVAIQALWAGGAESLQMTFLGRAAVLGQFQHPNILRLEG
VVTKSRPLMVLTEFMELGPLDSFLRQREGQFSSLQLVAMQRGVAAAMQYLSSFAFVHRSLSAHSVLV
NSHLVCKVARLGHSPQGPSCLLRWAAPEVIAHGKHTTSSDVWSFGILMWEVMSYGERPYWDMSEQEV
LNAIEQEFRLPPPPGCPPGLHLLMLDTWQKDRARRPHFDQLVAAFDKMIRKPDTLQAGGDPGERPSQ
ALLTPVALDFPCLDSPQAWLSAIGLECYQDNFSKFGLCTFSDVAQLSLEDLPALGITLAGHQKKLLH
HIQLLQQHLRQQGSVEV

SEQ ID NO: 67 is an exemplary codon-optimized nucleic acid sequence of a CD40-EphB6tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGACTGGTGATCGGCTCTATCCTGGGC
GCCCTGGCCTTCCTGCTCCTGGCGGCCATCACCGTGCTGGCTGTTGTGTTCCAACGAAAAAGACGGG
GCACCGGCTACACCGAGCAGCTCCAGCAGTACAGCAGCCCTGGCCTGGGCGTTAAGTACTACATCGA
CCCCAGCACCTACGAGGACCCCTGCCAGGCCATCCGGGAGCTGGCCCGGGAAGTGGACCCTGCCTAT
ATCAAGATCGAGGAAGTGATCGGCACAGGCAGCTTCGGCGAGGTCCGGCAGGGCAGACTGCAGCCTC
GGGGCCGCAGAGAGCAGACCGTGGCTATCCAGGCCCTGTGGGCCGGCGGAGCCGAGTCTCTGCAGAT
GACCTTCCTGGGCAGAGCCGCCGTGCTGGGCCAGTTTCAGCATCCTAACATCCTGCGGCTGGAAGGA
GTGGTGACCAAGTCCAGACCCCTGATGGTGCTGACCGAGTTCATGGAGCTGGGACCTCTGGACAGCT
TTCTGAGACAGAGAGAAGGCCAATTCTCCAGTCTGCAGCTGGTGGCCATGCAGCGGGGCGTGGCTGC
CGCTATGCAGTACCTGAGCAGCTTCGCCTTCGTGCACAGAAGCCTGTCTGCCCACAGCGTCCTGGTG
AACAGCCACCTGGTGTGCAAGGTGGCCAGGCTGGGACATTCTCCACAGGGCCCTAGCTGCCTGCTGA
GATGGGCTGCCCCTGAAGTGATCGCCCACGGCAAGCACACCACAAGCTCTGATGTGTGGTCCTTCGG
CATCCTGATGTGGGAAGTGATGAGCTACGGCGAAAGACCATACTGGGACATGAGCGAGCAGGAGGTC
CTGAACGCCATCGAGCAGGAGTTTAGACTGCCCCCCCCCCCCGGTTGTCCTCCAGGCCTGCACCTGC
TGATGCTGGACACATGGCAGAAGGACAGAGCCAGAAGACCTCACTTCGACCAGCTGGTGGCCGCTTT
TGATAAGATGATCAGAAAACCTGATACACTGCAGGCCGGAGGAGATCCTGGAGAGCGGCCTAGCCAG
GCCCTGCTCACCCCTGTGGCCCTGGACTTCCCTTGTCTGGATAGCCCCCAAGCCTGGCTGTCCGCCA
TTGGTCTCGAGTGCTACCAGGACAATTTCAGCAAGTTCGGCCTGTGCACCTTCAGCGACGTGGCCCA
GCTGAGCCTGGAAGATCTGCCTGCTCTGGGCATCACACTGGCTGGCCACCAGAAAAAGCTGCTGCAC
CACATTCAGCTGCTGCAGCAGCACCTTAGACAACAAGGCTCTGTGGAAGTG

SEQ ID NO: 68 is the amino acid sequence of an exemplary CD40-DDR1tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRILIGCLVA
IILLLLLIIALMLWRLHWRRLLSKAERRVLEEELTVHLSVPGDTILINNRPGPREPPPYQEPRPRGN
PPHSAPCVPNGSALLLSNPAYRLLLATYARPPRGPGPPTPAWAKPTNTQAYSGDYMEPEKPGAPLLP
PPPQNSVPHYAEADIVTLQGVTGGNTYAVPALPPGAVGDGPPRVDFPRSRLRFKEKLGEGQFGEVHL
CEVDSPQDLVSLDFPLNVRKGHPLLVAVKILRPDATKNARNDFLKEVKIMSRLKDPNIIRLLGVCVQ
DDPLCMITDYMENGDLNQFLSAHQLEDKAAEGAPGDGQAAQGPTISYPMLLHVAAQIASGMRYLATL
NFVHRDLATRNCLVGENFTIKIADFGMSRNLYAGDYYRVQGRAVLPIRWMAWECILMGKFTTASDVW
AFGVTLWEVLMLCRAQPFGQLTDEQVIENAGEFFRDQGRQVYLSRPPACPQGLYELMLRCWSRESEQ
RPPFSQLHRFLAEDALNTV

SEQ ID NO: 69 is an exemplary codon-optimized nucleic acid sequence of a CD40-DDR1tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATTCTGATCGGCTGTCTGGTGGCC
ATCATCCTGCTGCTGCTGCTGATCATCGCCCTGATGCTGTGGCGGCTTCACTGGAGAAGACTGCTGT
CTAAGGCCGAGCGGCGGGTGCTGGAAGAGGAACTGACAGTGCACCTCAGCGTGCCTGGCGACACCAT
CCTGATTAACAACAGACCAGGCCCTAGAGAGCCACCCCCCTACCAGGAGCCTAGACCCCGGGGCAAT
CCTCCTCACAGCGCCCCTTGCGTGCCAAATGGCAGCGCCCTGCTGCTGAGCAACCCCGCTTATAGAC
TGCTGCTGGCCACATACGCCAGACCTCCTAGAGGCCCCGGTCCTCCTACCCCTGCCTGGGCCAAGCC
TACAAACACCCAGGCCTACAGCGGCGACTACATGGAACCTGAGAAACCTGGCGCCCCTCTGCTGCCT
CCTCCACCACAGAACAGCGTGCCCCACTACGCAGAAGCCGACATCGTGACCTTGCAGGGCGTGACCG
GCGGCAACACCTACGCTGTTCCTGCCCTCCCCCCTGGCGCCGTGGGCGATGGACCACCTCGGGTGGA
TTTTCCTCGGAGCCGGCTGAGGTTTAAGGAAAAGCTGGGCGAGGGACAGTTTGGCGAAGTGCACCTG
TGCGAGGTGGACAGCCCTCAGGACCTGGTGTCCCTGGACTTCCCCCTGAACGTGCGGAAGGGCCACC
CCCTGCTGGTGGCAGTGAAAATCCTGAGACCCGACGCCACCAAGAACGCCAGAAACGACTTCCTGAA
AGAGGTGAAGATCATGAGCAGACTGAAGGACCCCAACATCATCAGACTGTTGGGCGTGTGCGTGCAG
GACGACCCTCTGTGTATGATCACCGATTACATGGAAAATGGCGACCTGAACCAGTTCCTGTCCGCCC
ACCAGCTCGAGGACAAGGCCGCTGAGGGCGCTCCTGGAGATGGCCAAGCTGCTCAAGGACCTACAAT
CAGCTACCCCATGCTGCTCCACGTGGCCGCCCAGATCGCCTCTGGCATGAGATACCTGGCCACACTG
AATTTCGTGCATAGAGATCTGGCCACCAGAAACTGCCTGGTTGGCGAAAACTTCACCATCAAGATCG
CCGATTTTGGAATGTCTCGCAATCTGTATGCCGGCGATTACTACAGAGTGCAGGGGAGAGCCGTGCT
GCCCATCCGGTGGATGGCCTGGGAGTGCATCCTGATGGGCAAGTTCACAACAGCCAGCGACGTGTGG
GCTTTCGGAGTTACCCTGTGGGAAGTCCTGATGCTGTGCAGAGCCCAACCTTTCGGCCAGCTGACCG
ACGAGCAGGTGATCGAGAACGCTGGCGAGTTCTTCAGAGACCAGGGCCGACAGGTGTACCTGTCTAG
ACCCCCTGCTTGTCCTCAGGGACTGTACGAGCTGATGCTGCGGTGCTGGTCCCGGGAGAGCGAGCAG
AGACCTCCATTCAGCCAGCTGCACAGATTCCTGGCTGAAGATGCCCTGAACACCGTC

SEQ ID NO: 70 is the amino acid sequence of an exemplary CD40-ROS1tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRTSFILTII
VGIFLVVTIPLTFVWHRRLKNQKSAKEGVTVLINEDKELAELRGLAAGVGLANACYAIHTLPTQEEI
ENLPAFPREKLTLRLLLGSGAFGEVYEGTAVDILGVGSGEIKVAVKTLKKGSTDQEKIEFLKEAHLM
SKFNHPNILKQLGVCLLNEPQYIILELMEGGDLLTYLRKARMATFYGPLLTLVDLVDLCVDISKGCV
YLERMHFIHRDLAARNCLVSVKDYTSPRIVKIGDFGLARDIYKNDYYRKRGEGLLPVRWMAPESLMD
GIFTTQSDVWSFGILIWEILTLGHQPYPAHSNLDVLNYVQTGGRLEPPRNCPDDLWNLMTQCWAQEP
DQRPTFHRIQDQLQLFRNFFLNSIYKSRDEANNSGVINESFEGEDGDVICLNSDDIMPVALMETKNR
EGLNYMVLATECGQGEEKSEGPLGSQESESCGLRKEEKEPHADKDFCQEKQVAYCPSGKPEGLNYAC
LTHSGYGDGSD

SEQ ID NO: 71 is an exemplary codon-optimized nucleic acid sequence of a CD40-ROS1tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAACAAGCTTTATCCTGACCATCATC
GTGGGCATCTTTCTGGTCGTCACCATCCCCCTGACCTTCGTGTGGCACCGGAGACTGAAAAACCAGA
AGAGCGCCAAGGAAGGCGTCACCGTGCTCATCAACGAGGATAAGGAGCTGGCTGAACTGCGGGGCCT
CGCTGCTGGCGTGGGATTGGCTAATGCCTGCTACGCCATCCACACCCTGCCAACTCAAGAAGAGATC
GAGAACCTGCCTGCCTTCCCTAGGGAAAAGCTGACCCTGAGACTGCTGCTCGGAAGCGGCGCCTTTG
GCGAGGTGTACGAGGGCACCGCCGTGGACATCCTGGGCGTGGGCAGCGGAGAGATTAAGGTGGCCGT
GAAGACCCTGAAGAAGGGCTCAACAGATCAGGAGAAGATCGAGTTCCTGAAGGAAGCACATCTGATG
AGCAAGTTCAACCACCCTAACATTCTGAAGCAGCTGGGCGTGTGCCTGCTGAACGAGCCTCAGTACA
TCATCCTGGAACTGATGGAAGGCGGAGATCTTCTGACATACCTGAGAAAGGCCAGAATGGCCACATT
CTACGGCCCTCTGCTGACACTGGTTGACCTGGTGGACCTGTGCGTGGACATCAGCAAGGGCTGTGTG
TATCTGGAACGGATGCACTTCATCCACAGAGACCTGGCCGCTAGAAATTGCCTGGTGTCCGTGAAAG
ATTACACCAGCCCTCGGATCGTGAAGATCGGCGATTTCGGCCTGGCCCGGGACATCTACAAGAACGA
CTACTACCGCAAAAGAGGCGAAGGACTGCTGCCAGTGCGGTGGATGGCCCCTGAGTCTCTGATGGAC
GGAATCTTCACCACCCAGAGCGACGTGTGGTCCTTCGGCATTCTGATCTGGGAAATCCTGACACTGG
GCCACCAGCCCTACCCCGCTCACAGCAACCTGGACGTGCTGAACTACGTGCAGACAGGCGGAAGACT
GGAACCCCCCAGAAACTGTCCTGATGATCTGTGGAACCTGATGACCCAGTGCTGGGCCCAGGAGCCT
GACCAAAGACCTACATTTCACAGAATCCAGGACCAGCTGCAACTGTTCAGAAACTTCTTCCTGAACA
GCATCTATAAGTCTCGGGACGAGGCCAACAACAGCGGCGTGATCAACGAGAGCTTCGAGGGCGAGGA
CGGAGATGTGATCTGCCTGAATAGCGACGACATCATGCCTGTTGCCCTGATGGAAACCAAGAACAGA
GAGGGCCTGAACTACATGGTGCTGGCCACCGAGTGCGGCCAGGGCGAGGAAAAAAGCGAGGGCCCTC
TGGGATCTCAGGAGTCCGAGAGCTGCGGCCTGAGAAAGGAGGAAAAGGAACCCCACGCCGACAAGGA
CTTCTGCCAGGAGAAACAGGTGGCCTACTGTCCTAGCGGTAAGCCCGAGGGCCTGAATTACGCCTGT
CTGACCCACTCTGGCTACGGCGACGGCTCTGAC

SEQ ID NO: 72 is the amino acid sequence of an exemplary CD40-ALKtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRVVTSALVA
ALVLAFSGIMIVYRRKHQELQAMQMELQSPEYKLSKLRTSTIMTDYNPNYCFAGKTSSISDLKEVPR
KNITLIRGLGHGAFGEVYEGQVSGMPNDPSPLQVAVKTLPEVCSEQDELDFLMEALIISKENHQNIV
RCIGVSLQSLPRFILLELMAGGDLKSFLRETRPRPSQPSSLAMLDLLHVARDIACGCQYLEENHFIH
RDIAARNCLLTCPGPGRVAKIGDFGMARDIYRASYYRKGGCAMLPVKWMPPEAFMEGIFTSKTDTWS
FGVLLWEIFSLGYMPYPSKSNQEVLEFVTSGGRMDPPKNCPGPVYRIMTQCWQHQPEDRPNFAIILE
RIEYCTQDPDVINTALPIEYGPLVEEEEKVPVRPKDPEGVPPLLVSQQAKREEERSPAAPPPLPTTS
SGKAAKKPTAAEISVRVPRGPAVEGGHVNMAFSQSNPPSELHKVHGSRNKPTSLWNPTYGSWFTEKP
TKKNNPIAKKEPHDRGNLGLEGSCTVPPNVATGRLPGASLLLEPSSLTANMKEVPLFRLRHFPCGNV
NYGYQQQGLPLEAATAPGAGHYEDTILKSKNSMNQPGP

SEQ ID NO: 73 is an exemplary codon-optimized nucleic acid sequence of a CD40-ALKtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAGTCGTGACCAGCGCCCTTGTGGCT
GCTCTGGTGCTGGCCTTCAGCGGCATCATGATCGTGTACCGCAGAAAGCACCAGGAGCTACAGGCCA
TGCAGATGGAACTGCAGAGCCCTGAATATAAGCTGAGCAAGCTGCGGACCAGCACCATCATGACCGA
CTACAACCCTAATTACTGCTTCGCCGGAAAGACCTCCAGCATCAGCGACCTGAAGGAAGTGCCTCGG
AAAAATATCACCCTGATCCGGGGCCTGGGCCACGGCGCTTTTGGAGAAGTGTACGAGGGCCAGGTGT
CCGGCATGCCTAACGACCCCAGCCCCCTGCAAGTGGCCGTGAAGACTCTGCCTGAGGTTTGTAGCGA
GCAGGATGAGCTGGACTTCCTGATGGAAGCCCTGATCATCTCTAAGTTCAACCACCAGAACATCGTG
CGGTGCATCGGCGTGTCACTGCAGTCTCTGCCTAGATTCATCCTGCTGGAGCTGATGGCCGGCGGCG
ACCTGAAGTCTTTTCTGAGAGAAACCAGACCTAGACCTAGCCAGCCTTCCAGCCTGGCCATGCTGGA
TCTGCTGCACGTGGCCCGGGACATCGCATGCGGCTGCCAGTACCTGGAAGAAAACCACTTTATCCAC
CGGGATATCGCCGCTAGGAACTGCCTGCTCACCTGTCCTGGCCCTGGCAGAGTGGCCAAGATCGGTG
ATTTCGGCATGGCCAGAGACATCTACCGGGCCTCTTACTACAGAAAGGGCGGCTGTGCCATGCTGCC
TGTGAAGTGGATGCCCCCCGAGGCCTTCATGGAAGGTATTTTCACAAGCAAAACAGACACATGGTCC
TTCGGCGTGCTGCTGTGGGAGATCTTCAGCCTGGGCTACATGCCTTACCCCTCTAAGAGCAACCAGG
AGGTCCTGGAGTTCGTGACCTCCGGAGGCAGAATGGACCCTCCAAAGAACTGCCCTGGACCTGTGTA
CAGAATCATGACCCAGTGCTGGCAGCACCAGCCTGAGGACAGACCGAATTTCGCCATCATTCTGGAA
AGAATCGAGTACTGTACCCAGGACCCCGACGTGATCAACACCGCCCTGCCTATCGAGTACGGACCTT
TAGTCGAGGAAGAGGAAAAGGTGCCCGTGCGGCCCAAGGACCCCGAGGGCGTTCCACCTCTGCTCGT
CAGCCAACAAGCTAAAAGAGAGGAGGAGAGAAGCCCTGCCGCTCCTCCACCTCTGCCCACAACCAGC
AGCGGCAAGGCCGCCAAGAAACCCACCGCCGCCGAGATTAGCGTGCGGGTGCCCAGAGGCCCCGCCG
TGGAAGGAGGACATGTGAACATGGCTTTTTCTCAGAGCAACCCTCCATCTGAGCTGCATAAGGTGCA
CGGCTCTAGAAACAAGCCTACCAGCCTGTGGAACCCTACATACGGCAGCTGGTTCACCGAGAAGCCT
ACCAAGAAGAACAACCCCATCGCTAAGAAGGAACCTCACGATAGAGGCAATCTGGGACTGGAGGGCA
GCTGCACAGTGCCCCCCAATGTGGCTACAGGCCGCCTGCCAGGCGCCAGCCTGCTGCTGGAGCCCAG
CTCCCTGACCGCCAACATGAAAGAGGTGCCTCTGTTCAGACTGAGACACTTCCCTTGCGGCAATGTG
AACTACGGATATCAGCAGCAAGGCCTGCCACTGGAAGCCGCCACAGCCCCTGGCGCCGGCCACTACG
AGGATACAATCCTGAAAAGCAAGAACAGCATGAACCAGCCTGGCCCC

SEQ ID NO: 74 is the amino acid sequence of an exemplary CD40-LTKtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRLVLMVAVV
ATSTLSLLMVCGVLILVKQKKWQGLQEMRLPSPELELSKLRTSAIRTAPNPYYCQVGLGPAQSWPLP
PGVTEVSPANVILLRALGHGAFGEVYEGLVIGLPGDSSPLQVAIKTLPELCSPQDELDELMEALIIS
KFRHQNIVRCVGLSLRATPRLILLELMSGGDMKSFLRHSRPHLGQPSPLVMRDLLQLAQDIAQGCHY
LEENHFIHRDIAARNCLLSCAGPSRVAKIGDFGMARDIYRASYYRRGDRALLPVKWMPPEAFLEGIF
TSKTDSWSFGVLLWEIFSLGYMPYPGRTNQEVLDFVVGGGRMDPPRGCPGPVYRIMTQCWQHEPELR
PSFASILERLQYCTQDPDVLNSLLPMELGPTPEEEGTSGLGNRSLECLRPPQPQELSPEKLKSWGGS
PLGPWLSSGLKPLKSRGLQPQNLWNPTYRS

SEQ ID NO: 75 is an exemplary codon-optimized nucleic acid sequence of a CD40-LTKtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGACTGGTGCTGATGGTCGCTGTTGTG
GCCACAAGCACCCTGAGCCTGCTGATGGTGTGCGGCGTGCTGATCCTTGTCAAACAGAAAAAGTGGC
AGGGCCTGCAGGAAATGCGGCTGCCGTCTCCTGAGCTGGAACTGAGCAAGCTGAGAACATCCGCCAT
TAGAACCGCCCCTAACCCTTACTACTGCCAGGTGGGCCTGGGCCCCGCTCAGAGCTGGCCTCTGCCT
CCTGGCGTGACCGAGGTGTCGCCCGCCAACGTGACCCTGCTGAGGGCCCTGGGCCACGGCGCCTTTG
GCGAGGTGTACGAGGGCCTTGTGATCGGCCTGCCTGGCGATAGCTCCCCACTGCAAGTGGCAATCAA
GACACTGCCCGAGTTGTGCAGCCCTCAAGACGAGCTGGATTTCCTGATGGAAGCCCTGATCATCAGC
AAGTTCCGGCACCAGAACATCGTGCGGTGCGTGGGACTCAGCCTGAGAGCTACACCTAGACTGATCC
TGCTGGAACTGATGAGCGGAGGAGATATGAAGTCTTTTCTGCGCCACAGCCGGCCACATCTGGGCCA
GCCTAGCCCTCTGGTGATGAGAGACCTGCTGCAGCTGGCCCAGGATATCGCCCAGGGATGTCACTAC
CTGGAGGAAAACCACTTCATCCACCGGGACATCGCCGCTAGAAACTGCCTGCTGAGCTGCGCCGGCC
CATCTAGAGTGGCCAAGATTGGCGACTTCGGCATGGCCAGAGATATCTACCGGGCTTCATATTACCG
TAGAGGCGACAGAGCCCTGCTGCCTGTGAAGTGGATGCCCCCTGAGGCCTTCCTGGAAGGCATCTTC
ACCTCCAAGACCGACAGCTGGTCCTTCGGAGTGCTCCTGTGGGAGATCTTCAGCCTGGGCTACATGC
CTTATCCTGGAAGAACCAACCAGGAGGTCCTGGACTTCGTGGTGGGCGGAGGCAGAATGGACCCCCC
CAGAGGCTGTCCTGGCCCCGTGTACCGGATCATGACCCAGTGCTGGCAACACGAGCCTGAGCTGAGA
CCCAGCTTTGCCAGCATCCTGGAAAGACTGCAGTACTGTACCCAGGACCCCGACGTGCTGAACAGCC
TGCTGCCTATGGAACTTGGCCCCACCCCTGAAGAAGAGGGTACAAGCGGCCTCGGCAATAGATCTCT
GGAGTGCCTGCGGCCTCCACAGCCTCAGGAGCTCAGCCCCGAGAAGCTGAAGAGCTGGGGCGGATCT
CCATTAGGTCCTTGGCTGAGTTCTGGCCTGAAACCTCTGAAGTCCCGGGGCCTGCAGCCTCAAAATC
TGTGGAACCCCACATACAGAAGC

SEQ ID NO: 76 is the amino acid sequence of an exemplary CD40-CD11atm_CD18 chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRYLYVLSGI
GGLLLLLLIFIVLYKVGFFKRNLKEKMEAGRGVPNGIPAEDSEQLASGQEAGDPGCLKPLHEKDSES
GGGKDRAKRSGSGATNFSLLKQAGDVEENPGPMVRLPLQCVLWGCLLTAVHPEPPTACREKQYLINS
QCCSLCQPGQKLVSDCTEFTETECLPCGESEFLDTWNRETHCHQHKYCDPNLGLRVQQKGTSETDTI
CTCEEGWHCTSEACESCVLHRSCSPGFGVKQIATGVSDTICEPCPVGFFSNVSSAFEKCHPWTSCET
KDLVVQQAGTNKTDVVCGPQDRLRIAAIVGGTVAGIVLIGILLLVIWKALIHLSDLREYRRFEKEKL
KSQWNNDNPLFKSATTTVMNPKFAES

SEQ ID NO: 77 is an exemplary codon-optimized nucleic acid sequence of a CD40-CD11atm_CD18tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGATATCTGTACGTGCTGTCCGGCATC
GGCGGCCTGCTGCTGCTGCTCCTGATCTTCATCGTGCTGTACAAAGTGGGGTTTTTCAAGCGGAACC
TCAAGGAAAAGATGGAAGCTGGCAGAGGCGTCCCCAACGGCATCCCTGCCGAAGATAGCGAGCAGCT
GGCCAGCGGACAGGAGGCCGGAGATCCTGGCTGTCTTAAGCCTCTGCACGAGAAGGACAGCGAGAGC
GGCGGCGGAAAGGATAGAGCCAAGAGAAGCGGCTCTGGAGCCACAAATTTCAGCCTGCTGAAACAAG
CCGGCGACGTGGAAGAGAACCCCGGCCCTATGGTGCGCCTGCCTCTGCAGTGCGTCCTGTGGGGCTG
CTTACTGACCGCCGTGCACCCTGAGCCACCTACAGCCTGTAGAGAAAAACAGTACCTGATCAACAGC
CAATGTTGCAGCCTGTGCCAGCCTGGCCAGAAGCTGGTTTCTGATTGCACCGAGTTCACCGAGACAG
AGTGCCTTCCATGCGGAGAAAGCGAGTTCCTGGACACCTGGAATAGAGAGACCCACTGTCACCAGCA
CAAGTACTGCGACCCTAACCTGGGCCTGAGGGTGCAGCAGAAAGGCACATCCGAGACAGATACCATC
TGCACCTGTGAAGAGGGCTGGCACTGCACATCTGAAGCCTGCGAGTCTTGTGTGCTGCACCGGAGCT
GCAGCCCTGGCTTCGGCGTGAAGCAGATCGCTACCGGCGTGAGCGACACCATCTGCGAGCCCTGTCC
TGTGGGCTTCTTTTCTAATGTGAGCTCTGCCTTCGAAAAGTGCCATCCCTGGACCAGCTGCGAGACC
AAGGACCTGGTGGTCCAGCAAGCTGGAACCAACAAGACCGACGTGGTGTGCGGCCCCCAGGACAGAC
TGAGAATCGCCGCTATCGTGGGCGGCACCGTGGCCGGCATCGTGCTGATTGGAATCCTGCTGCTGGT
GATCTGGAAGGCCCTGATCCACCTGTCCGACCTGCGGGAATACCGGAGATTCGAGAAGGAAAAACTG
AAGTCCCAGTGGAACAACGACAACCCCCTGTTTAAGAGCGCCACTACCACAGTGATGAACCCTAAGT
TCGCCGAGAGC

SEQ ID NO: 78 is the amino acid sequence of an exemplary CD40-CD16Atm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRVSFCLVMV
LLFAVDTGLYFSVKTNIRSSTRDWKDHKFKWRKDPQDK

SEQ ID NO: 79 is an exemplary codon-optimized nucleic acid sequence of a CD40-CD16Atm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAGTGTCCTTCTGCCTGGTGATGGTC
CTGCTGTTTGCCGTGGACACCGGCCTGTACTTCAGCGTGAAGACAAACATCAGATCTAGCACCAGAG
ATTGGAAGGACCACAAGTTCAAGTGGCGGAAAGATCCTCAGGACAAG

SEO ID NO: 80 is the amino acid sequence of an exemplary CD40-NKG2Dtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRPFFFCCFI
AVAMGIRFIIMVTMGWIRGRRSRHSWEMSEFHNYNLDLKKSDFSTRWQKQRCPVVKSKCRENAS

SEQ ID NO: 81 is an exemplary codon-optimized nucleic acid sequence of a CD40-NKG2Dtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGACCCTTCTTTTTCTGCTGTTTTATC
GCCGTGGCTATGGGCATCCGGTTCATCATCATGGTCACAATGGGCTGGATCAGAGGCAGACGGAGCA
GACACAGCTGGGAAATGAGCGAGTTCCACAACTACAACCTGGACCTGAAGAAGTCCGATTTCAGCAC
AAGATGGCAGAAACAGCGGTGCCCTGTGGTGAAGAGCAAGTGCAGAGAGAATGCTTCT

SEQ ID NO: 82 is the amino acid sequence of an exemplary CD40-CD94tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRLTAEVLGI
ICIVLMATVLKTIVLMNKQRGTFSEVSLAQDPKRQQRKPKGNKSSISGTEQEIFQVELNLQNPSLNH
QGIDKIYDCQGLLPPPEK

SEQ ID NO: 83 is an exemplary codon-optimized nucleic acid sequence of a CD40-CD94tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGACTCACAGCTGAAGTGCTGGGCATC
ATCTGCATCGTCCTGATGGCCACAGTGCTGAAGACCATCGTGCTGATGAACAAGCAGCGGGGCACCT
TCAGCGAGGTGTCCCTGGCCCAGGACCCCAAAAGACAGCAGAGAAAGCCTAAGGGCAACAAAAGCTC
TATCAGCGGCACCGAGCAGGAGATCTTCCAAGTGGAACTGAATCTGCAGAACCCCAGCCTGAACCAC
CAGGGAATTGATAAGATCTACGACTGTCAGGGCCTGCTGCCTCCACCTGAGAAG

SEQ ID NO: 84 is the amino acid sequence of an exemplary CD40-NKp46tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRMGLAFLVL
VALVWFLVEDWLSRKRTRERASRASTWEGRRRLNTQTL

SEQ ID NO: 85 is an exemplary codon-optimized nucleic acid sequence of a CD40-NKp46tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATGGGCCTGGCCTTCCTCGTGCTG
GTGGCCCTGGTGTGGTTCCTGGTCGAGGACTGGCTGAGCAGAAAGCGGACCCGGGAAAGAGCTTCTA
GAGCCAGCACCTGGGAGGGCAGACGGAGACTGAACACCCAGACACTG

SEQ ID NO: 86 is the amino acid sequence of an exemplary CD40-NKp30tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRAGTVLLLR
AGFYAVSFLSVAVGSTVYYQGKCLTWKGPRRQLPAVVPAPLPPPCGSSAHLLPPVPGG

SEQ ID NO: 87 is an exemplary codon-optimized nucleic acid sequence of a CD40-NKp30tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAGCTGGCACCGTGCTGCTCCTGAGA
GCCGGCTTCTACGCCGTGTCCTTCCTGAGCGTGGCCGTGGGCAGCACCGTCTACTACCAGGGCAAGT
GCCTGACATGGAAGGGCCCCAGACGGCAGCTGCCCGCTGTGGTGCCTGCCCCTCTGCCTCCTCCATG
TGGCTCTAGCGCCCACCTGCTGCCTCCTGTGCCCGGAGGA

SEQ ID NO: 88 is the amino acid sequence of an exemplary CD40-NKp44tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRLVPVFCGL
LVAKSLVLSALLVWWGDIWWKTMMELRSLDTQKATCHLQQVTDLPWTSVSSPVEREILY
HTVARTKISDDDDEHTL

SEQ ID NO: 89 is an exemplary codon-optimized nucleic acid sequence of a CD40-NKp44tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGACTCGTCCCCGTGTTCTGCGGCCTG
CTGGTGGCCAAGTCTCTGGTGCTGAGCGCCCTGCTGGTTTGGTGGGGCGACATCTGGTGGAAGACCA
TGATGGAACTGCGGAGCCTGGACACCCAGAAAGCCACATGTCACCTGCAGCAGGTGACCGACCTGCC
TTGGACCAGCGTGTCCAGCCCTGTGGAAAGAGAGATCCTGTACCACACCGTGGCTAGAACAAAGATC
AGCGACGATGATGACGAGCACACACTG

SEQ ID NO: 90 is the amino acid sequence of an exemplary CD40-NKp80tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRILLGISGT
VNGILTLTLISLIMQDEERYMTLNVQSKKRSSAQTSQLTFKDYSVTLHWYK

SEQ ID NO: 91 is an exemplary codon-optimized nucleic acid sequence of a CD40-NKp80tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATCCTGCTGGGCATCAGCGGAACC
GTGAACGGCATCCTGACACTCACACTGATCTCCCTGATCATGCAAGATGAGGAACGGTACATGACCC
TGAACGTGCAGAGCAAGAAAAGATCTAGCGCCCAGACCAGCCAGCTGACCTTCAAGGACTACAGCGT
GACCCTGCACTGGTACAAG

SEQ ID NO: 92 is the amino acid sequence of an exemplary CD40-KIR2DS1tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRVLIGTSVV
KIPFTILLFFLLHRWCSDKKNAAVMDQEPAGNRTVNSEDSDEQDHQEVSYA

SEQ ID NO: 93 is an exemplary codon-optimized nucleic acid sequence of a CD40-KIR2DS1tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAGTCCTGATCGGCACAAGCGTGGTG
AAGATCCCCTTCACCATCCTGCTGTTTTTCCTGCTGCACCGGTGGTGCTCTGATAAGAAAAACGCCG
CTGTGATGGACCAGGAGCCTGCCGGCAATAGAACCGTGAACAGCGAAGATAGCGACGAGCAGGACCA
CCAGGAAGTGTCCTACGCC

SEQ ID NO: 94 is the amino acid sequence of an exemplary CD40-CLEC2Dtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRFFLIMFLT
IIVCGMVAALSAIMHDSNNVEKDITPSELPANPGCLHSKEHSIKATLIWRL

SEQ ID NO: 95 is an exemplary codon-optimized nucleic acid sequence of a CD40-CLEC2Dtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGATTTTTCCTGATCATGTTCCTGACC
ATCATCGTGTGCGGCATGGTGGCCGCCCTGAGCGCCATCATGCACGATAGCAACAACGTGGAAAAGG
ACATCACCCCTAGCGAGCTGCCTGCTAATCCCGGCTGTCTGCACTCCAAAGAGCACTCTATCAAGGC
CACACTGATTTGGAGACTG

SEO ID NO: 96 is the amino acid sequence of an exemplary CD40-KIR3DS1tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRILIGTSVV
KIPFTILLFFLLHRWCSNKKKCCCNGPRACREQK

SEQ ID NO: 97 is an exemplary codon-optimized nucleic acid sequence of a CD40-KIR3DS1tm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATCCTGATCGGCACATCTGTGGTG
AAGATCCCCTTCACCATCCTGCTGTTTTTCCTGCTGCACCGGTGGTGCAGCAACAAGAAAAAGTGCT
GTTGCAACGGCCCTAGAGCCTGCAGAGAGCAGAAG

SEQ ID NO: 98 is the amino acid sequence of an exemplary CD40-CD28tm_YMFM chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRFWVLVVVG
GVLACYSLLVTVAFIIFWVRSKRSRLLHSDYMFMTPRRPGPTRKHYQPYAPPRDFAAYRS

SEQ ID NO: 99 is an exemplary codon-optimized nucleic acid sequence of a CD40-CD28tm_YMFM chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGATTCTGGGTGCTGGTGGTGGTGGGA
GGCGTGCTGGCCTGCTACTCTCTGCTGGTGACCGTGGCCTTCATCATCTTTTGGGTGAGGAGCAAGA
GGTCCCGCCTGCTGCACAGCGACTATATGTTCATGACCCCTCGGAGACCAGGCCCCACAAGAAAGCA
CTACCAGCCCTATGCCCCCCCTCGGGATTTTGCCGCCTACAGAAGC

SEQ ID NO: 100 is the amino acid sequence of an exemplary CD40-4-1BBtm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRIISFFLAL
TSTALLFLLFFLTLRFSVVKRGRKKLLYIFKQPFMRPVQTTQEEDGCSCRFPEEEEGGCEL

SEQ ID NO: 101 is an exemplary codon-optimized nucleic acid sequence of a CD40-4-1BBtm chimera:

ATGGCGAGGCTGCCACTGCAGTGCGTGCTGTGGGGATGTCTGCTGACCGCAGTGCACCCAGAGCCCC
CTACAGCCTGCCGCGAGAAGCAGTACCTGATCAACAGCCAGTGCTGTTCCCTGTGCCAGCCTGGCCA
GAAGCTGGTGAGCGACTGTACCGAGTTCACCGAGACAGAGTGCCTGCCATGTGGCGAGTCCGAGTTT
CTGGACACCTGGAACCGGGAGACACACTGCCACCAGCACAAGTATTGTGATCCCAATCTGGGCCTGA
GAGTGCAGCAGAAGGGCACCTCTGAGACCGATACAATCTGCACATGTGAGGAGGGCTGGCACTGCAC
CTCTGAGGCATGCGAGAGCTGCGTGCTGCACCGGTCTTGTAGCCCCGGCTTCGGCGTGAAGCAGATC
GCCACCGGCGTGTCCGACACAATCTGCGAGCCATGTCCCGTGGGCTTCTTTTCTAACGTGAGCTCCG
CCTTTGAGAAGTGCCACCCTTGGACCTCCTGTGAGACAAAGGATCTGGTGGTGCAGCAGGCCGGCAC
CAATAAGACAGACGTGGTGTGCGGACCTCAGGATAGGCTGAGAATCATCTCCTTCTTCCTGGCCCTG
ACATCTACCGCCCTGCTGTTCCTGCTGTTTTTTCTCACACTGAGATTCAGCGTGGTGAAGCGGGGCA
GAAAGAAACTGCTGTACATCTTCAAGCAGCCTTTCATGCGGCCTGTGCAGACCACCCAGGAGGAGGA
CGGCTGCAGCTGCAGATTCCCCGAGGAAGAGGAAGGCGGATGTGAACTG

SEQ ID NO: 102 is the amino acid sequence of an exemplary CD40-TLR4tm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRTIIGVSVL
SVLVVSVVAVLVYKFYFHLMLLAGCIKYGRGENIYDAFVIYSSQDEDWVRNELVKNLEEGVPPFQLC
LHYRDFIPGVAIAANIIHEGFHKSRKVIVVVSQHFIQSRWCIFEYEIAQTWQFLSSRAGIIFIVLQK
VEKTLLRQQVELYRLLSRNTYLEWEDSVLGRHIFWRRLRKALLDGKSWNPEGTVGTGCNWQEATSI

SEQ ID NO: 103 is an exemplary codon-optimized nucleic acid sequence of a CD40-TLR4tm chimera:

ATGGTGAGGCTGCCACTGCAGTGCGTGCTGTGGGGATGTCTGCTGACCGCAGTGCACCCAGAGCCAC
CAACAGCCTGTCGCGAGAAGCAGTACCTGATCAATTCTCAGTGCTGTAGCCTGTGCCAGCCCGGCCA
GAAGCTGGTGTCCGATTGTACCGAGTTCACCGAGACAGAGTGCCTGCCTTGTGGCGAGTCTGAGTTT
CTGGATACCTGGAACCGGGAGACACACTGCCACCAGCACAAGTATTGTGACCCCAATCTGGGCCTGA
GAGTGCAGCAGAAGGGCACCTCCGAGACCGACACAATCTGCACATGTGAGGAGGGATGGCACTGCAC
CAGCGAGGCATGCGAGTCCTGCGTGCTGCACCGCTCCTGTTCTCCCGGCTTCGGCGTGAAGCAGATC
GCCACCGGCGTGTCTGATACAATCTGCGAGCCCTGTCCTGTGGGCTTCTTTAGCAACGTGTCCTCTG
CCTTTGAGAAGTGCCACCCTTGGACCAGCTGTGAGACAAAGGACCTGGTGGTGCAGCAGGCCGGCAC
CAATAAGACAGATGTGGTGTGCGGACCTCAGGACAGGCTGAGAACCATCATCGGCGTGAGCGTGCTG
TCCGTGCTGGTGGTGTCTGTGGTGGCCGTGCTGGTGTACAAGTTCTACTTTCACCTGATGCTGCTGG
CCGGCTGCATCAAGTATGGCAGGGGCGAGAACATCTACGACGCCTTCGTGATCTATAGCTCCCAGGA
CGAGGATTGGGTGCGCAACGAGCTGGTGAAGAATCTGGAGGAGGGCGTGCCCCCTTTCCAGCTGTGC
CTGCACTACAGGGACTTCATCCCCGGCGTGGCAATCGCCGCCAATATCATCCACGAGGGCTTTCACA
AGTCCAGGAAAGTGATCGTGGTGGTGTCCCAGCACTTCATCCAGTCTCGCTGGTGTATCTTTGAGTA
TGAGATCGCCCAGACCTGGCAGTTCCTGTCTAGCCGGGCCGGCATCATCTTTATCGTGCTGCAGAAG
GTGGAGAAGACCCTGCTGAGACAGCAGGTGGAGCTGTACAGGCTGCTGAGCCGCAACACATATCTGG
AGTGGGAGGACTCCGTGCTGGGCCGGCACATCTTCTGGCGGAGACTGAGAAAGGCCCTGCTGGATGG
CAAGTCTTGGAACCCAGAGGGCACCGTGGGCACAGGATGTAATTGGCAGGAGGCCACAAGCATC

SEQ ID NO: 104 is the amino acid sequence of an exemplary CD40-CD40Ltm chimera:

MVRLPLQCVLWGCLLTAVHPEPPTACREKQYLINSQCCSLCQPGQKLVSDCTEFTETECLPCGESEF
LDTWNRETHCHQHKYCDPNLGLRVQQKGTSETDTICTCEEGWHCTSEACESCVLHRSCSPGFGVKQI
ATGVSDTICEPCPVGFFSNVSSAFEKCHPWTSCETKDLVVQQAGTNKTDVVCGPQDRLRLYVAFLAS
GIMQTILFVTLLYMFIKMSIPLGTAASRPSTQNYTEIM

SEQ ID NO: 105 is an exemplary codon-optimized nucleic acid sequence of a CD40-CD40Ltm chimera:

ATGGTGAGGCTGCCACTGCAGTGCGTGCTGTGGGGATGTCTGCTGACCGCAGTGCACCCAGAGCCAC
CAACAGCCTGTCGCGAGAAGCAGTACCTGATCAATTCTCAGTGCTGTAGCCTGTGCCAGCCCGGCCA
GAAGCTGGTGTCCGATTGTACCGAGTTCACCGAGACAGAGTGCCTGCCTTGTGGCGAGTCTGAGTTT
CTGGATACCTGGAACCGGGAGACACACTGCCACCAGCACAAGTATTGTGACCCCAATCTGGGCCTGA
GAGTGCAGCAGAAGGGCACCTCCGAGACCGACACAATCTGCACATGTGAGGAGGGATGGCACTGCAC
CAGCGAGGCATGCGAGTCCTGCGTGCTGCACCGCTCCTGTTCTCCCGGCTTCGGCGTGAAGCAGATC
GCCACCGGCGTGTCTGATACAATCTGCGAGCCCTGTCCTGTGGGCTTCTTTAGCAACGTGTCCTCTG
CCTTTGAGAAGTGCCACCCTTGGACCAGCTGTGAGACAAAGGACCTGGTGGTGCAGCAGGCCGGCAC
CAATAAGACAGATGTGGTGTGCGGACCTCAGGACAGGCTGAGACTGTACGTGGCCTTCCTGGCCAGC
GGCATCATGCAGACCATCCTGTTCGTGACACTGCTGTATATGTTTATCAAGATGTCTATCCCACTGG
GCACCGCAGCCAGCCGGCCTTCCACCCAGAACTACACAGAGATCATG

### DETAILED DESCRIPTION

### I. Terms

Unless otherwise specifically explained, all technical and scientific terms used herein have the same meaning as commonly understood by those of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Definitions of common terms in molecular biology can be found in, for example, Lewin's Genes X, Jones & Bartlett Publishers, 2009 (ISBN 10 0763766321); Krebs et al. (eds.), The Encyclopedia of Molecular Biology, Blackwell Science Ltd., 1994 (ISBN 0-632-02182-9); Meyers R. A. (ed.), Molecular Biology and Biotechnology: A Comprehensive Desk Reference, VCH Publishers, Inc., 1995 (ISBN 1-56081-569-8); and George P. Rédei, Encyclopedic Dictionary of Genetics, Genomics, Proteomics and Informatics, 3^{rd} Edition, Springer, 2008 (ISBN: 1402067534), and other similar references.

Although methods and materials similar or equivalent to those described herein can be used in the practice or testing of the present disclosure, suitable methods and materials are described below. All publications, patent applications, patents, and other references mentioned herein are incorporated by reference in their entirety. All database accession numbers (such as GenBank or UniProt accession numbers) are incorporated herein by reference in their entirety. In case of conflict, the present specification, including explanations of terms, will control. In addition, the materials, methods, and examples are illustrative only and not intended to be limiting.

In order to facilitate review of the various embodiments of the disclosure, the following explanations of specific terms are provided:

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A single-chain antibody (scFv) is a genetically engineered molecule containing the V_{H} and V_{L} domains of one or more antibody (ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi: 10.1155/2012/980250; Marbry, IDrugs, 13:543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) may be used. In a dsFv the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, IL); Kuby, J., Immunology, 3^{rd} Ed., W.H. Freeman & Co., New York, 1997.

Autologous: Refers to tissues, cells or nucleic acids taken from an individual's own tissues. For example, in an autologous transfer or transplantation of modified T cells described herein, the donor and recipient are the same person. Therapies utilizing engineered autologous T cells have the advantage of being patient-specific, as the therapeutic T cells are created from a patient's own cells.

Typically, to produce an autologous T cell for a therapeutic formulation or use, an individual's peripheral blood mononuclear cells (PBMCs) are collected, and then subjected to ex vivo genetic modification. CD8+ T cells are isolated from the collected PBMCs, activated, and transduced with a construct encoding, for example, a CD40 protein, or a CD40 protein and a TCR to produce the autologous T cell, which may then be culture-expanded, formulated, and optionally cryopreserved.

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrated to other parts of the body, for example via the bloodstream or lymph system.

CD40 Protein: As used herein, “CD40 protein” refers to both wild-type CD40 proteins and chimeric CD40-derived proteins comprising a CD40 extracellular domain. In some embodiments, a CD40 protein is a chimeric CD40-derived protein further comprising a heterologous intracellular signaling domain, a heterologous transmembrane domain, or both.

Chimeric antigen receptor (CAR): A chimeric molecule that includes an antigen-binding portion (such as a single domain antibody or single-chain variable fragment (scFv)) and an intracellular signaling domain, such as a signaling domain having an immunoreceptor tyrosine-based activation motif (ITAM); for example, CD3ζ (which transduces TCR-mediated signals from the surface of a T cell in adaptive immune response), or FcεRIγ (which mediates allergic inflammatory signaling in mast cells). Typically, CARs additionally include a transmembrane domain, anchoring the CAR in a host cell membrane. A CAR transmembrane domain may be derived from, for example, any of CD3, CD4, CD8, and CD28. A CAR intracellular signaling domain may further include at least one co-stimulatory domain; for example, any of CD28, 4-1BB (CD137), ICOS, OX40 (CD134), CD27, and DAP10. In one specific example, the intracellular signaling domain includes two co-stimulatory domains linked to CD34 (e.g., CD28 and 4-1BB). In some examples, a CAR may still further comprise a JAK-STAT activation domain (e.g., a JAK-STAT activation domain derived from IL-2RB), which domain may be included within the practitioner's discretion to stimulate cell proliferation and/or prevent terminal differentiation in particular applications.

T cells expressing a CAR are referred to as “CAR-T” cells. For effecting selective apoptosis of tumor cells, the T cell is transduced with a construct directing the expression of a CAR comprising a tumor-specific antigen. Binding to the tumor antigen activates the intracellular CD33 domain to initiate downstream intracellular events in the T cell and ultimately tumor-specific cytotoxicity. CAR-T cells may be derived from T cells obtained from either a patient (autologous) or from a healthy donor (allogeneic).

Complementarity determining region (CDR): A region of hypervariable amino acid sequence that defines the binding affinity and specificity of an antibody. The light and heavy chains of a mammalian immunoglobulin each have three CDRs, designated VL-CDR1, VL-CDR2, VL-CDR3 and VH-CDR1, VH-CDR2, VH-CDR3, respectively.

Ectopic: The term “ectopic,” as applied to molecules herein (for example, CD40 proteins, TCRs, and polynucleotides encoding the same), refers to a polypeptide/protein or polynucleotide in a specific environment or context in which it is not normally present. For example, if a host cell is transformed with a polynucleotide that does not occur in the untransformed host cell in nature, then that polynucleotide is ectopic to the host cell. By way of further example, if a modified host cell expresses a protein (e.g., CD40 proteins and/or TCRs) that does not occur in the wild-type host cell, or that occurs in the wild-type host cell under a different set of conditions, the protein is ectopic to the host cell. Accordingly, ectopic proteins (such as CD40 or TCRs) herein specifically include proteins that are identical in amino acid sequence to a protein already present in a host cell, but that are expressed in a different cellular context than the protein with the same sequence already present in the host cell; for example, wherein the ectopic protein is constitutively expressed or expressed under different conditions. Similarly, ectopic polynucleotides encoding the proteins herein specifically include polynucleotides that are identical in sequence to those already present in a host cell, but that are located in a different cellular or genomic context than the polynucleotide with the same sequence already present in the host cell. For example, a polynucleotide that is located in a different location in the host cell than a polynucleotide with the same sequence is normally integrated in the of the host cell (for example, as a component of an expression construct integrated at a different genomic locus than the polynucleotide of the same sequence is normally found) is ectopic to the host cell. Furthermore, a polynucleotide that is present in a plasmid or vector in the host cell is ectopic to the host cell when a polynucleotide with the same sequence is only normally present in the genome of the host cell.

Expression: As used herein, “expression” of a polynucleotide refers to the process by which the coded information of a transcriptional unit is converted into a polypeptide. The expression of a coding sequence can be influenced by external signals; for example, exposure of a cell, tissue, or organism to a signal (for example, an agonist or antigen that binds a cellular receptor) that increases or decreases gene expression. When used herein with regard to a polypeptide, “expression” refers to the synthesis of the polypeptide by a cell from a polynucleotide that encodes the polypeptide, for example, under the control of a regulatory element such as a promoter that is functional in the cell.

Heterologous: The term “heterologous,” as applied to polypeptides and/or polynucleotides herein, means of different origin. For example, if a host cell is transformed with a polynucleotide that does not occur in the untransformed host cell in nature, then that polynucleotide is heterologous (and ectopic) to the host cell. Similarly, if a host cell expresses a polypeptide (e.g., CD40 proteins and TCRs) that does not occur in the host cell in nature, then that polypeptide is heterologous (and ectopic) to the host cell. Furthermore, different elements (e.g., promoters, enhancers, coding sequences, and terminators) of an expression cassette may be heterologous to one another and/or to the transformed host cell. Heterologous polynucleotides herein also specifically include a polynucleotide that is identical in sequence to a polynucleotide already present in a host cell, but that is linked to a different regulatory sequence and/or is present at a different copy number in the host cell.

Inhibiting, ameliorating, or treating a disease: “Inhibiting” a condition refers to inhibiting the full development of a condition or disease, for example, cancer or a tumor. Inhibition of a condition occurs within the spectrum from partial inhibition to substantially complete inhibition of the disease. In some examples, the term “inhibiting” refers to reducing or delaying the onset or progression of a condition. “Ameliorating” refers to the reduction in the number or severity of signs or symptoms of a disease, such as cancer. “Treating” refers to a therapeutic intervention that decreases or inhibits a sign or symptom of a disease or pathological condition after it has begun to develop, such as a reduction in tumor size or tumor burden. A subject to be administered an effective amount of the disclosed modified T cells can be identified by standard diagnosing techniques for such a disorder, for example, presence of the disease or disorder or risk factors to develop the disease or disorder.

Isolated: An “isolated” biological component (such as a polynucleotide, protein, or cell) has been substantially separated, produced apart from, or purified away from other biological components (e.g., other cells, chromosomal and extra-chromosomal DNA and RNA, and proteins). Cells, polynucleotides, and proteins that have been isolated specifically include cells, nucleic acid molecules, and proteins purified by standard purification methods. The term also embraces nucleic acid molecules and proteins prepared by recombinant expression or production in a host organism, as well as chemically-synthesized nucleic acid molecules.

Pharmaceutically acceptable carriers: The pharmaceutically acceptable carriers of use are known to those of ordinary skill in the art. Remington: The Science and Practice of Pharmacy, 22^{nd} ed., London, UK: Pharmaceutical Press, 2013, describes compositions and formulations suitable for pharmaceutical delivery of the disclosed agents. In general, the nature of the carrier will depend on the particular mode of administration being employed. For example, parenteral formulations usually include injectable fluids that include pharmaceutically and physiologically acceptable fluids such as water, physiological saline, balanced salt solutions, aqueous dextrose, glycerol or the like as a vehicle.

In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as in a selected quantity in a vial. Unit dosage forms can include one dosage or multiple dosages (for example, in a vial from which metered dosages of the agent can selectively be dispensed).

Purified: The term purified does not require absolute purity; rather, it is intended as a relative term. Thus, for example, a purified nucleic acid, protein or cell preparation is one in which the nucleic acid, protein, or cell is more enriched than in its original environment. In one embodiment, a preparation is purified such that the nucleic acid, protein, or cells represent at least 50% of the total nucleic acid, protein, or cell content of the preparation.

Recombinant: A recombinant nucleic acid is one that has a sequence that is not naturally occurring or has a sequence that is made by an artificial combination of two otherwise separated segments of sequence (such as a chimeric nucleic acid). This artificial combination can be accomplished by chemical synthesis or by the manipulation of isolated segments of nucleic acids, for example, by genetic engineering techniques. In some examples, a chimeric protein is encoded by a recombinant nucleic acid. In several embodiments, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid, or the nucleic acid can be integrated into the host cell chromosome.

Regulatory element: “Regulatory elements,” and “regulatory sequences” refer to nucleic acid elements that influence the timing and level/amount of transcription (or RNA processing or stability) of an operably linked polynucleotide. Particular regulatory elements may be located upstream and/or downstream of a polynucleotide operably linked thereto. Also, particular regulatory elements operably linked to a polynucleotide may be located on the associated complementary strand of a double-stranded nucleic acid molecule. Regulatory elements include, for example and without limitation, promoters, translation leaders, introns, enhancers, stem-loop structures, repressor binding sequences, termination sequences, and polyadenylation recognition sequences. In examples herein, a polynucleotide is operably linked to a promoter, such that the polynucleotide is expressed in a host cell. Specific promoters herein utilized include “constitutive” promoters, which refers to a promoter that is active under most or all conditions in a host cell. In other examples, a polynucleotide is operably linked to an “inducible” promoter, which refers to a promoter wherein the rate of transcription increases in response to an inducing agent (for example, a signal molecule).

Specific binding: As used herein in the context of intermolecular interactions, a polypeptide exhibits “specific” binding (or binds “specifically”) to another molecule when contact between the polypeptide and the molecule results in a stable intermolecular interaction under physiological in vivo or ex vivo conditions, wherein contact between the polypeptide and other molecules does not result in a stable intermolecular interaction under the same conditions. For example, an extracellular ligand binding domain of a receptor binds specifically to a ligand when contact between the ligand binding domain of the receptor and the ligand results in a stable intermolecular interaction such that downstream signaling is initiated by the receptor, wherein the extracellular ligand binding domain of the receptor does not form a stable interaction with other molecules such that signaling is initiated. By way of further example, a recognition domain polypeptide of a receptor binds specifically to an antigen when contact between the recognition domain polypeptide of the receptor and the antigen results in a stable intermolecular interaction such that downstream signaling is initiated by the receptor, wherein the recognition domain polypeptide of the receptor does not form a stable interaction with other antigens such that signaling is initiated. For example, a recognition domain polypeptide of a TCR specifically binds a neoantigen when the recognition domain polypeptide of the TCR stably binds the neoantigen and initiates intracellular signaling events, but does not stably bind the antigen from which the neoantigen is derived such that the intracellular signaling is initiated.

Subject: Living multi-cellular vertebrate organisms, a category that includes human and non-human mammals, such as non-human primates, pigs, sheep, cows, dogs, cats, rodents, and the like. In an example, a subject is a human.

T cell: A white blood cell (lymphocyte) that is an important mediator of the immune response. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8⁺ T cell is a cytotoxic T lymphocyte (CTL). In another embodiment, a CD8⁺ cell is a suppressor T cell.

Activated T cells can be detected by an increase in cell proliferation and/or expression of or secretion of one or more cytokines (such as IL-2, IL-4, IL-6, IFNγ, or TNFα). Activation of CD8+ T cells can also be detected by an increase in cytolytic activity in response to an antigen.

In some examples, a “modified T cell” is a T cell transduced or transfected with a heterologous nucleic acid (such as one or more of the nucleic acids or vectors disclosed herein) or expressing one or more heterologous proteins (such as an ectopic CD40 protein or a chimeric CD40 protein). The terms “modified T cell” and “transduced T cell” are used interchangeably in some examples herein.

Transformation/Transduction: As used herein, the terms “transformation” and “transduction” are used interchangeably to refer to the transfer of one or more polynucleotide(s) into a cell. A cell is “transformed” or “transduced” by a polynucleotide when a nucleic acid molecule comprising the polynucleotide is introduced into the cell, and the polynucleotide becomes stably replicated by the cell, either by incorporation of the polynucleotide into the cellular genome, or by episomal replication. As used herein, the transformation and transduction encompass all techniques by which a nucleic acid molecule can be introduced into such a cell. Examples include, but are not limited to, transduction with viral vectors, electroporation, microinjection, direct DNA uptake, and microprojectile bombardment.

Transgene: The term “transgene” refers to an ectopic polynucleotide in the genome of an organism. In some examples, a transgene comprises a nucleotide sequence encoding a polypeptide (for example, CD40 proteins and/or TCRs). In these and other examples, a transgene may contain regulatory elements (for example, a constitutive promoter or inducible promoter) operably linked to a nucleotide sequence encoding a polypeptide.

Vector: Vectors include nucleic acid molecules as introduced into a cell, for example, to produce a transformed cell. A vector may include genetic elements that permit it to replicate in the host cell. Examples of vectors include but are not limited to plasmids, viruses, and linear DNA molecules that carry ectopic nucleic acids into a cell under suitable conditions. A vector may include one or more coding polynucleotides, including those that encode selectable or screenable marker genes (for example, fluorescent molecules such as GFP) and/or other genetic elements known in the art. A vector may transduce, transform, or infect a cell, thereby causing the cell to express RNA molecules and/or polypeptides encoded by the vector. A vector optionally includes materials to aid in achieving entry of the nucleic acid molecule into the cell (e.g., a liposome, protein coating, etc.).

### II. CD40 Proteins

Disclosed herein are CD40 proteins that include a functional CD40 extracellular domain, such that intracellular signaling is activated in a T cell by the binding of a CD40 agonist (for example, CD40L or an agonistic antibody against CD40). Such CD40 proteins are referred to in some instances herein as “a functional ectopic CD40 protein.”

In some embodiments, a CD40 protein includes a full-length CD40 protein, such as a full-length human CD40 protein. In some examples, the CD40 protein has at least 95% sequence identity (such as at least 95%, at least 96%, at least 97%, at least 98%, or at least 99% sequence identity) with the amino acid sequence of SEQ ID NO:1. In other examples, the CD40 protein includes or consists of the amino acid sequence of SEQ ID NO: 1.

In other embodiments, the CD40 protein is a chimeric CD40 protein that includes a CD40 extracellular domain and at least one heterologous intracellular domain. The CD40 extracellular domain has the function of specifically binding a ligand, such as the native CD40L or an agonistic CD40 antibody. In particular embodiments, the CD40 extracellular domain is a human CD40 extracellular domain; e.g., a polypeptide that is about 193 amino acids in length (for example, between 174-212, between 185-200, between 190-195, 192, 193, and 194 amino acids in length), which in some examples includes an amino acid sequence that is at least 90%, at least 95%, at least 98%, at least 99%, or 100% identical to SEQ ID NO: 2.

The chimeric CD40 proteins provided herein further include a transmembrane domain. In some embodiments, the transmembrane domain is a CD40 transmembrane domain. In particular embodiments, the CD40 transmembrane domain is a human CD40 transmembrane domain; e.g., a polypeptide that is about 22 amino acids in length (for example, between 19-25, between 21-23, and 22 amino acids in length), which in some examples, includes an amino acid sequence that is at least 85%, at least 90%, at least 95%, or 100% identical to SEQ ID NO: 3. In other examples, the transmembrane domain is a heterologous transmembrane domain, such as a transmembrane domain from the same protein as the heterologous intracellular domain.

The chimeric CD40 proteins provided herein also include at least one heterologous intracellular domain, for example, a heterologous intracellular signaling domain, such that intracellular signaling mediated by the heterologous domain is activated upon binding of CD40L or an agonistic anti-CD40 antibody. In some examples, the intracellular domain (or portion thereof) is from a member of the CD28 family (such as CD28 or ICOS), CD2, CD4, CD8, a cytokine receptor, a member of the TNFR family, a TNF ligand, a member of the TLR family, a member of the receptor tyrosine kinase (RTK) family, a member of the integrins family, or a member of the KIR family.

Exemplary CD28 family members include CD28 (e.g., GenBank Accession No. NP_006130.1) and ICOS (also known as CD278; e.g., GenBank Accession No. NP_036224.1). Additional intracellular domains may be from CD2 (e.g., GenBank Accession No. NP_001758.2), CD4 (e.g., GenBank Accession No. NP_000607.1), or CD8 (also known as CD8A; e.g., GenBank Accession No. NP_001759.3).

Exemplary cytokine receptors include colony stimulating factor 2 receptor subunit beta (CSF2RB, e.g., GenBank Accession No. NP_000386.1), colony stimulating factor 2 receptor subunit alpha (CSF2Ra, also referred to as GMCSFRa, e.g., GenBank Accession Nos. NP_758448.1, NP_758449.1, NP_758450.1, or NP_758452.1), interlukin-3 receptor subunit alpha (IL3Ra, e.g., GenBank Accession No. NP_002174.1 or NP_001254642.1), interleukin-2 receptor subunit beta (IL2Rb, e.g., GenBank Accession No. NP_000869.1), interleukin-2 receptor subunit gamma (IL2Rg, also referred to as common gamma chain, e.g., GenBank Accession No. NP_000197.1), interleukin-9 receptor (IL9R, e.g., GenBank Accession Nos. NP_002177.2 or NP_789743.2), interleukin-7 receptor subunit alpha (IL7Ra, e.g., GenBank Accession No. NP_002176.2), or cytokine receptor like factor 2 (CRLF2, also known as TSLPR, e.g., GenBank Accession No. NP_071431.2 or NP_001012288.2). Exemplary members of the TNFR family include 4-1BB (also known as TNF receptor superfamily member 9 (TNFRSF9), e.g., GenBank Accession No. NP_001552.2), OX40 (also known as TNF receptor superfamily member 4 (TNFRSF4), e.g., GenBank Accession No. NP_003318.1), CD27 (e.g., GenBank Accession No. NP_001233.2), CD30 (also known as TNF receptor superfamily member 8 (TNFRSF8), e.g., GenBank Accession No. NP_001234.3 or NP_001268359.2), GITR (also known as TNF receptor superfamily member 18 (TNFRSF18), e.g., GenBank Accession No. NP_004186.1, NP_683699.1, or NP_683700.1), or TNFR2 (also known as TNF receptor superfamily member 1B (TNFRSF1B), e.g., GenBank Accession No. NP_001057.1).

Exemplary TNF ligands include RANKL (also known as TNF superfamily member 11, e.g., GenBank Accession No. NP_003692.1 or NP_143026.1), LIGHT (also known as tumor necrosis factor ligand superfamily member 14 (TNFSF14) e.g. GenBank Accession No. NP_00798.2 or NP_742011.2), FasL (Fas ligand, e.g., GenBank Accession No. NP_000630.1 or NP_001289675.1), 41BBL (also known as TNF superfamily member 9 (TNFSF9), e.g., GenBank Accession No. NP_003802.1), CD30L (also known as TNF superfamily member 8 (TNFSF8), e.g., GenBank Accession No. NP_001235.1 or NP_001239219.1), CD27L (also known as CD70, e.g., GenBank Accession No. NP_001243.1 or NP_001317261.1), OX40L (also known as GNF superfamily member 4 (TNFSF4), e.g., GenBank Accession No. NP_003317.1 or NP_001284491.1), TWEAK (also known as GNF superfamily member 12 (TNFSF12), e.g., GenBank Accession No. NP_003800.1), BAFF (also known as TNF superfamily member 13b (TNFSF13B), e.g., GenBank Accession No. NP_006564.1 or NP_001139117.1), TRAIL (also known as TNF superfamily member 10 (TNFSF10), e.g., GenBank Accession No. NP_003801.1 or NP_001177871.1), EDA (ectodysplasin A, e.g., GenBank Accession No. NP_001390.1), or GITRL (also known as TNF superfamily member 18 (TNFSF18), e.g., GenBank Accession No. NP_005083.3).

Exemplary members of the TLR family include TLR1 (e.g., GenBank Accession No. NP_003254.2), TLR2 (e.g., GenBank Accession No. NP_003255.2), TLR3 (e.g., GenBank Accession No. NP_003256.1), TLR4 (e.g., GenBank Accession No. NP_612564.1, NP_003257.1, or NP_612567.1), TLR5 (e.g., GenBank Accession No. NP_003259.2), TLR6 (e.g., GenBank Accession No. NP_006059.2), TLR7 (e.g., GenBank Accession No. NP_057646.1), TLR8 (e.g., GenBank Accession No. NP_057694.2 or NP_619542.1), TLR9 (e.g., GenBank Accession No. NP_059138.1), or TLR10 (e.g., GenBank Accession No. NP_112218.2 or NP_001182037.1).

Exemplary members of the receptor tyrosine kinase (RTK) family include IGF1R (e.g., GenBank Accession No. NP_000866.1), EGFR (also known as ERBB1, e.g., GenBank Accession No. NP_001333826.1), TRKA (also known as NTRK1, e.g., GenBank Accession No. NP_001007793.1, NP_001012331.1, or NP_002520.2), TRKB (also known as NTRK2, e.g., GenBank Accession No. NP_001007098.1), TRKC (also known as NTRK3, e.g., GenBank Accession No. NP_001007157.1), MUSK (e.g., GenBank Accession No. NP_001159752.1), MET (also known as c-Met, e.g., GenBank Accession No. NP_000236.2), AXL (e.g., GenBank Accession No. NP_001265528.1), TIE2 (also known as TEK, e.g., GenBank Accession No. NP_000450.3), EPHA1 (e.g., GenBank Accession No. NP_005223.4), EPHA3 (e.g., GenBank Accession No. NP_005224.2), EPHA7 (e.g., GenBank Accession No. NP_001275558.1), EPHA8 (e.g., GenBank Accession No. NP_001006944.1), EPHB1 (e.g., GenBank Accession No. NP_004432.1), EPHB2 (e.g., GenBank Accession No. NP_001296121.1), EPHB3 (e.g., GenBank Accession No. NP_004434.2), EPHB6 (e.g., GenBank Accession No. NP_001267723.2), DDR1 (e.g., GenBank Accession No. NP_001189450.1), ALK (e.g., GenBank Accession No. NP_001340694.1), or LTK (also known as TYK1, e.g., GenBank Accession No. NP_001129157.1).

Exemplary members of the integrins family include CD11A (also known as ITGAL or LFA-1, e.g., GenBank Accession No. NP_001107852.1), or CD18 (also known as ITGB2, e.g., GenBank Accession No. NP_000202.3).

Exemplary members of the human killer immunoglobulin-like receptor (KIR) family include NKG2D (also known as KLRK1, e.g., GenBank Accession No. NP_031386.2), CD94 (also known as KLRD1, e.g., GenBank Accession No. NP_001107868.2), NKP46 (also known as NCR1, e.g., GenBank Accession No. NP_001138929.2), NKP30 (also known as NCR3, e.g., GenBank Accession No. NP_001138938.1), NKP44 (also known as NCR2, e.g., GenBank Accession No. NP_001186438.1), NKP80 (also known as KLRF1, e.g., GenBank Accession No. NP_001278751.1), KIR2DS1 (e.g., GenBank Accession No. NP_055327.1), CLEC2D (e.g., GenBank Accession No. NP_001004419.1), or KIR3DS1 (e.g., GenBank Accession No. NP_001077008.1).

In some examples, the heterologous intracellular signaling domain includes a polypeptide including an amino acid sequence that is, at least 90%, at least 95%, at least 98%, at least 99%, or 100% identical to a sequence selected from SEQ ID NOs: 5-13. In other examples, the heterologous intracellular signaling domain includes a polypeptide including an amino acid sequence that is at least 90%, at least 95%, at least 98%, at least 99%, or 100% identical to a sequence selected from amino acids 218-625 of SEQ ID NO: 34; amino acids 217-758 of SEQ ID NO: 36; amino acids 210-566 of SEQ ID NO: 38; amino acids 218-585 of SEQ ID NO: 40; amino acids 218-603 of SEQ ID NO: 42; amino acids 215-567 of SEQ ID NO: 44; amino acids 217-651 of SEQ ID NO: 46; amino acids 215-636 of SEQ ID NO: 48; amino acids 215-1168 of SEQ ID NO: 50; amino acids 215-622 of SEQ ID NO: 52; amino acids 218-635 of SEQ ID NO: 54; amino acids 215-636 of SEQ ID NO: 56; amino acids 215-656 of SEQ ID NO: 58; amino acids 217-656 of SEQ ID NO: 60; amino acids 215-705 of SEQ ID NO: 62; amino acids 215-632 of SEQ ID NO: 64; amino acids 215-620 of SEQ ID NO: 66; amino acids 215-689 of SEQ ID NO: 68; amino acids 217-681 of SEQ ID NO: 70; amino acids 215-775 of SEQ ID NO: 72; amino acids 219-633 of SEQ ID NO: 74; amino acids 215-273 and 517-562 of SEQ ID NO: 76; amino acids 215-239 of SEQ ID NO: 78; amino acids 215-265 of SEQ ID NO: 80; amino acids 217-286 of SEQ ID NO: 82; amino acids 215-239 of SEQ ID NO: 84; amino acids 215-259 of SEQ ID NO: 86; amino acids 215-277 of SEQ ID NO: 88; amino acids 215-252 of SEQ ID NO: 90; amino acids 213-252 of SEQ ID NO: 92; amino acids 215-252 of SEQ ID NO: 94; amino acids 214-235 of SEQ ID NO: 96; amino acids 221-261 of SEQ ID NO: 98; amino acids 221-262 of SEQ ID NO: 100; amino acids 215-401 of SEQ ID NO: 102; or amino acids 218-239 of SEQ ID NO: 104.

In one embodiment, the chimeric CD40 protein includes a CD40 extracellular domain, a transmembrane domain (such as a CD40 transmembrane domain or a CD28 transmembrane domain), and a CD28 intracellular signaling domain. In other embodiments, the chimeric CD40 protein includes a CD40 extracellular domain, a transmembrane domain (such as a CD40 transmembrane domain or a GMCSFRa transmembrane domain), and a GMCSFRa intracellular signaling domain; or a CD40 extracellular domain, a transmembrane domain (such as a CD40 transmembrane domain or a CSF2Rb transmembrane domain), and a CSF2Rb intracellular signaling domain. In further embodiments, the chimeric CD40 protein includes a CD40 extracellular domain, a transmembrane domain (such as a CD40 transmembrane domain or a IL2Rb transmembrane domain), and a IL2Rb intracellular signaling domain; or a CD40 extracellular domain, a transmembrane domain (such as a CD40 transmembrane domain or a IL2Rg transmembrane domain), and a IL2Rg (common gamma chain) intracellular signaling domain. In additional examples, the chimeric CD40 protein includes a CD40 extracellular domain, a transmembrane domain (such as a CD40 transmembrane domain or a CD28 transmembrane domain), and a CD28 intracellular signaling domain with YMFM mutation. In other examples, the chimeric CD40 protein includes a CD40 extracellular domain, a transmembrane domain (such as a CD40 transmembrane domain or a IGF1R transmembrane domain), and an IGF1R intracellular signaling domain. In further examples, the chimeric CD40 protein includes a CD40 extracellular domain, a transmembrane domain (such as a CD40 transmembrane domain or a 4-1BB transmembrane domain), and a 4-1BB intracellular signaling domain. In still other examples, the chimeric CD40 protein includes a CD40 extracellular domain, a transmembrane domain (such as a CD40 transmembrane domain or a TLR4 transmembrane domain), and a TLR4 intracellular signaling domain.

In some examples, the chimeric CD40 protein includes a polypeptide including an amino acid sequence that is, at least 90%, at least 95%, at least 98%, at least 99%, or 100% identical to a sequence selected from any one of SEQ ID NOs: 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, or 104.

Nucleic acids encoding the disclosed chimeric CD40 proteins are also provided. In some embodiments, the nucleic acid is codon-optimized, for example for expression in human cells. Methods of codon-optimization are known to one of skill in the art. In some examples, the chimeric CD40 protein is encoded by a nucleic acid including a nucleic acid sequence that is, at least 90%, at least 95%, at least 98%, at least 99%, or 100% identical to a sequence selected from any one of SEQ ID NOs: 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, or 105.

### III. Modified T Cells Expressing Ectopic CD40 Proteins

Also disclosed herein are T cells modified to express at least one ectopic CD40 protein, including one or more of the full-length and chimeric CD40 proteins described in Section II. In one example, the modified T cells express a chimeric CD40 protein including a CD40 extracellular domain, a transmembrane domain, and a CD28 intracellular signaling domain. In some embodiments, the modified T cells express two or more different ectopic CD40 proteins, for example, such that the two intracellular domains generate a signal in response to ligand binding. In one example, the modified T cells express a chimeric CD40 protein including a CD40 extracellular domain, a transmembrane domain (such as a CD40 transmembrane domain or a GMCSFRa transmembrane domain), and a GMCSFRa intracellular signaling domain and a chimeric CD40 protein including a CD40 extracellular domain, a transmembrane domain, (such as a CD40 transmembrane domain or a CSF2Rb transmembrane domain) and a CSF2Rb intracellular signaling domain. In another example, the modified T cells express a chimeric CD40 protein including a CD40 extracellular domain, a transmembrane domain (such as a CD40 transmembrane domain or a IL2Rb transmembrane domain), and a IL2Rb intracellular signaling domain and a chimeric CD40 protein including a CD40 extracellular domain, a transmembrane domain (such as a CD40 transmembrane domain or a IL2Rg transmembrane domain), and a IL2Rg (common gamma chain) intracellular signaling domain.

The modified T cells are in some examples, tumor-reactive T cells. Thus, in some examples, the modified T cells express at least one tumor-reactive TCR, which in some examples, is an ectopic or heterologous TCR. In other examples, the modified T cells herein express at least one ectopic CD40 protein (including one or more of the full-length and chimeric CD40 proteins described in Section II) and a chimeric antigen receptor.

The modified T cell may be, for example, a human T cell. In particular examples, the modified T cell is a T cell from a subject with cancer, for example, that is to be administered to the subject in an autologous T cell immunotherapy. In some examples, the T cell is derived from peripheral blood lymphocytes from a subject. In other examples, the T cell is derived from tumor-reactive tumor infiltrating lymphocytes (TIL) from a subject with cancer.

Binding of a CD40 agonist, such as CD40L and agonistic anti-CD40 antibodies, to a modified T cell according to embodiments herein initiates signal transduction in the modified T cell. In particular embodiments, CD40 agonist binding activates the modified T cell to produce an adaptive immune response mediated by the modified T cell, and/or induces APCs to initiate an innate immune response. In some examples, the modified T cell effects both a cell-mediated adaptive immune response and an innate immune response via APC stimulation. To further increase the tumor cytotoxicity of modified T cells including an ectopic CD40 protein, the modified T cells may be modified to further express a TCR of interest or a CAR.

Cytotoxic CD8⁺ T cells can eliminate tumor cells through recognition of peptide epitopes presented on major histocompatibility complex class I (MHC-I) molecules by the alpha-beta T cell receptor (αβTCR). Each TCR chain contains three complementarity-determining regions (CDRs) generated by V (D) J recombination, which form six flexible loops that contact MHC-I. In general, the germline-encoded CDR1 and CDR2 contact the MHC-I heavy chain, while the hypervariable CDR3 binds the MHC-I-bound peptide. In some embodiments, a modified T cell including an ectopic CD40 protein further includes an ectopic TCR including a CDR3 domain that specifically binds a peptide derived from a tumor-associated antigen, a cancer-testis antigen, a viral antigen of a virally-derived tumor cell, or a neoantigen. Neoantigens are peptides derived from mutated “self” proteins that the immune system detects as “nonself.” Many neoantigens are characteristic features of individual tumors, and some embodiments herein provide T cells that comprise a TCR with a CDR3 domain that specifically binds a neoantigen; for example, a neoantigen derived from a tumor in a subject from whom the transduced host T cell was obtained, or a neoantigen that is commonly found in a tumor cell type to be targeted by the modified T cell.

Particular embodiments include a modified T cell expressing a TCR that specifically binds a neoantigen derived from a common or “hotspot” mutation found in tumor cells, such as those arising in the RAS proteins and p53. The RAS family (H, N, and KRAS) of small GTPases are among the most commonly mutated oncogenes in cancer, found in ˜27% of tumors. Among them, the G12D mutation in KRAS occurs most frequently and is found in ˜45% of pancreatic, 13% of colorectal, and 4% of lung cancers. In particular embodiments, a modified T cell includes an ectopic CD40 protein (such as one or more of the full-length and chimeric CD40 proteins described in Section II) and a TCR (such as an ectopic TCR) that specifically binds a tumor-derived antigen peptide, for example, a neoantigen peptide, and in specific examples, KRASG12D

Also provided are compositions and formulations including the modified T cells herein described. Some embodiments provide a composition or formulation including a population of modified T cells and a pharmaceutically acceptable carrier.

The pharmaceutically acceptable carriers of use are known to those of ordinary skill in the art. Remington: The Science and Practice of Pharmacy, 22″d ed., London, UK: Pharmaceutical Press, 2013, describes compositions and formulations suitable for pharmaceutical delivery of the disclosed agents. In general, the nature of the carrier will depend on the particular mode of administration being employed. For example, parenteral formulations usually include injectable fluids that include pharmaceutically and physiologically acceptable fluids such as water, physiological saline, balanced salt solutions, aqueous dextrose, glycerol or the like as a vehicle.

In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some embodiments, the active agent and pharmaceutically acceptable carrier are provided in a unit dosage form such as in a selected quantity in a vial. Unit dosage forms can include one dosage or multiple dosages (for example, in a vial from which metered dosages of the agent can selectively be dispensed). In further embodiments, the compositions or formulation includes a population of modified T cells and cryogenic media or cell culture media.

In particular embodiments, the compositions or formulation includes about 10⁴ to 10¹² of the modified T cells (for example, about 10⁴-10⁸ cells, about 10⁶-10⁸ cells, or about 10⁶-10¹² cells). In specific examples, the composition includes at least about 10⁴, at least about 10⁵, at least about 10⁶, or at least about 10⁷ modified T cells.

In some embodiments, the composition further includes a CD40 agonist. In some examples, the CD40 agonist is CD40L. In other examples, the CD40 agonist is an agonistic anti-CD40 antibody. By way of non-limiting example, the agonistic anti-CD40 antibody may be CDX-1140 (Celldex Therapeutics, Hampton, NJ), CP-870,893 (selicrelumab), ADC-1013 (mitazalimab), or SGN-40 (dacetuzumab). A composition including a modified T cell and an agonistic anti-CD40 antibody may still further include a cancer vaccine vector, for example, wherein the activity of the agonistic antibody on the CD40 protein provides adjuvant activity with respect to the cancer vaccine.

The foregoing compositions and formulations may comprise autologous modified T cells with respect to an intended subject for administration. In other examples, however, the compositions and formulations comprise allogenic T cells that may be administered to a subject from which the host T cells were not isolated.

### IV. Methods of Making Modified T Cells Expressing Ectopic CD40 Proteins

Modified T cells of embodiments herein may be generated by transducing host T cells (for example CD8⁺ or CD4⁺ T cells) with a nucleic acid molecule comprising at least one polynucleotide encoding a CD40 protein that comprises a functional CD40 extracellular domain (such as a full-length CD40 protein or a chimeric CD40 protein), wherein the polynucleotide is operably linked to a promoter that is functional in the host T cell. In some embodiments, the polynucleotide encodes a wild-type human CD40 protein, and it is used to transduce a human host T cell. In other embodiments, the polynucleotide encodes, for example, a chimeric CD40 protein, and the operably linked promoter directs its expression under the same or different conditions or amounts as an endogenous CD40 protein. In these and other embodiments, the operably linked promoter directs expression of the CD40 protein in the host T cell under conditions or in amounts that are measurably distinct from that of endogenous CD40 in the host T cell.

The promoter may include a constitutive promoter or an inducible promoter. Exemplary constitutive promoters include cytomegalovirus (CMV), SV40, phosphoglycerate kinase (PGK), ubiquitin C (UBC), elongation factor-1 (EFS), chicken β-actin short promoter (CBH), EF-1 alpha (EF1a) promoter, or EF1a short promoter, and certain hybrid promoters (e.g., a CMV enhancer fused to chicken β-actin promoter (CAG). In another example, the promoter is a promoter that is active when a TCR is triggered, such as an NFAT promoter. In other examples, the promoter is an inducible promoter.

In some embodiments, T cells are transduced with a vector comprising a polynucleotide encoding the CD40 protein, for example, a viral vector. In further embodiments, genome editing tools (for example, CRISPR/Cas9) are utilized to integrate the polynucleotide into the genome of the cell. Viral vectors suitable for transduction of T cells include lentivirus, retrovirus (e.g., γ-retrovirus), adenovirus, adeno-associated virus, vaccinia virus, and fowlpox vectors. In particular examples, T cells are transduced with a gamma retroviral vector comprising a polynucleotide encoding the CD40 protein. In other examples, T cells are transduced with lentiviral vectors comprising a polynucleotide encoding the CD40 protein. Some advantages of using a lentiviral system include long-term expression of the transgene, the ability to transduce both dividing cells and non-dividing cells, the ability to deliver complex genetic elements, lack of expression of viral proteins after transduction, lack of insertional mutagenesis in human cells, high titer production, and ease of vector manipulation and production.

Additional regulatory elements that may be included in a viral vector include enhancers, leader sequences, transcription terminators, start and/or stop codons, internal ribosome entry sites (IRES), splicing signals, and polyadenylation signals. In examples where the vector or construct includes two (or more) expression cassettes, the polynucleotides may be operably linked to (for example, separated by) an IRES or other multicistronic element, such as a P2A and/or T2A element. In some examples, the vector includes both a CD40 protein encoding polynucleotide and a polynucleotide encoding a TCR (e.g., TCR a and B subunits) or a CAR. In examples herein, the viral vector may also contain additional elements, for example and without limitation, packaging signals (e.g., lentivirus ų packaging signal), a central polypurine tract (cPPT), a woodchuck hepatitis virus post-transcriptional regulatory element (WPRE), and a Rev Response element (RRE). In particular examples, the lentivirus vector is self-inactivating.

Lentivirus vectors comprising a polynucleotide encoding a CD40 protein can be prepared by one of ordinary skill in the art utilizing known molecular biology techniques. For example, the polynucleotide can be cloned into a lentivirus transfer vector. Lentivirus plasmid systems (such as 3 or 4 plasmid systems) are commercially available, for example, from Clontech™ (Mountain View, CA), ThermoFisher™ Scientific (Waltham, MA), and Addgene™ (Cambridge, MA).

Host T cells for transduction using any of the foregoing techniques may be either directly isolated from human peripheral blood mononuclear cells (PBMCs) of a subject, or acquired from a vendor supplying, for example, allogeneic T cells for use in immunotherapy. PBMCs are a mixture of mononuclear cells comprising lymphocytes, monocytes, and dendritic cells. Lymphocytes (T cells, B cells, and NK cells) comprise 70-90% of the PBMCs, in amounts that may vary based on attributes like age, ethnicity, gender, disease state, and/or prior therapeutic interventions (for example, chemotherapy and radiotherapy).

In some embodiments wherein T cells are directly isolated from PMBCs, the PMBCs are separated from other blood components by leukapheresis, whereby whole blood from a subject or donor is passed through an apheresis machine that collects leukocytes or white blood cells, removes lymphocytes, and returns the remaining cells and plasma to the subject or donor. PBMCs may alternatively be manually isolated from other blood components in a whole blood sample by techniques known in the art. In one example, a whole blood sample is layered over a density-gradient medium and centrifuged to yield, inter alia, an opaque, white PBMC fraction. The PBMC fraction may then be removed from the gradient, for example, by gentle pipetting, and then be washed in culture medium or phosphate-buffered saline to remove residual density medium and any remaining platelets.

T cells may be isolated from a PBMC fraction by culture in a T-cell medium and stimulation with OKT3 and IL-2 to yield activated T cells for retroviral transduction. After viral transduction, the resulting modified T cells may be further cultured in IL-2 containing T-cell media and assayed (for example, 10-14 days post-transduction) for expression of the ectopic CD40 protein. Alternatively, CD8⁺ T cells may be isolated from PBMCs by labeling with biotinylated anti-CD8 mAb, and mixing the PBMCs containing the labeled CD8⁺ cells with secondary anti-biotin mAb magnetic particles. The CD8⁺ cells may then be isolated by passing the mixture through a magnetized column. In another example, CD4⁺ T cells may be isolated from PBMCs by labeling with biotinylated anti-CD4 mAb, and mixing the PBMCs containing the labeled CD4⁺ cells with secondary anti-biotin mAb magnetic particles. The CD4⁺ cells may then be isolated by passing the mixture through a magnetized column.

In other examples, the T cells are isolated from tumor-infiltrating lymphocytes (TIL). TILs are often enriched in tumor-reactive T cells since some of them are recognizing and mounting an immune response against the tumor. Thus, genetically engineering “bulk” TIL to express CD40 proteins may enhance therapeutic activity of TIL therapy since the CD40 proteins would be expressed by tumor-reactive TIL. In some examples, bulk TIL are isolated by processing tumor specimens (such as a tumor sample from a subject) into single-cell suspension. In some examples, this cell suspension is modified to express the CD40 proteins. Alternatively, TIL are first expanded from the tumor (either from a tumor fragment or single-cell suspension), followed by modification to express a CD40 protein. T cells may be expanded using T-cell growth factors such as IL-2, IL-7, IL-15, and IL-21, or a combination of these cytokines. Although bulk TIL often contain tumor-reactive T cells, their frequency can still be low. Therefore, in some embodiments, tumor-reactive T cells are enriched from bulk TIL populations. For example, T cells expressing markers such as PD-1, Tim-3, LAG3, and 4-1BB, as well as CD39 and CD103 are often tumor reactive. Thus, T cells expressing one or more of these markers can be isolated (for example, by FACS) and then the enriched population modified to express CD40 proteins.

In other embodiments, tumor-reactive T cells from the blood are enriched, for example using an in vitro stimulation process using a specific tumor antigen to selectively stimulate antigen-reactive T cells. For example, peripheral blood T cells can be stimulated with a tumor-antigen peptide (such as a neoantigen peptide) to selectively stimulate the low frequency tumor-reactive T cells. After one or two stimulation cycles, tumor-reactive T cells (if present) are enriched. These T cells are then modified to express the CD40 proteins, or further enriched by methods such as FACS, prior to modification. Alternatively, tumor-reactive T cells from either a tumor sample or blood may be enriched using MHC-tetramer technology to specifically stain and sort tumor-antigen-reactive T cells.

Modified T cells expressing a functional ectopic CD40 protein as herein described, for example, obtained by the foregoing methods are suitable for use in adoptive immunotherapy.

### V. Methods of Treatment

Methods of treating a subject (such as a subject with cancer) with the modified T cells disclosed herein are provided. In some embodiments, the methods include administering modified T cells expressing an ectopic CD40 protein and a TCR (for example, wherein the modified T cell further comprises an ectopic TCR) to a subject with cancer. In some embodiments, the modified T cells are administered intravenously, for example, in a parenteral formulation. In alternative embodiments, the modified T cells are delivered via intratumoral administration, which may improve anti-tumor efficacy and reduce adverse events secondary to immune activation. Other routes of administration can also be used.

In some embodiments, the formulations comprising modified T cells herein described are administered to a subject with cancer; for example, a solid tumor. Examples of solid tumors, include sarcomas (e.g., fibrosarcoma, myxosarcoma, liposarcoma, chondrosarcoma, osteogenic sarcoma, and other sarcomas), synovioma, mesothelioma, Ewing sarcoma, leiomyosarcoma, rhabdomyosarcoma, colon cancer, colorectal cancer, peritoneal cancer, esophageal cancer, pancreatic cancer, breast cancer (e.g., basal breast carcinoma, ductal carcinoma, and lobular breast carcinoma), lung cancer, ovarian cancer, prostate cancer, liver cancer (e.g., hepatocellular carcinoma), gastric cancer, squamous cell carcinoma (e.g., head and neck squamous cell carcinoma), basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, hepatoma, bile duct carcinoma, choriocarcinoma, Wilms tumor, cervical cancer, fallopian tube cancer, testicular tumor, seminoma, bladder cancer, kidney cancer (e.g., renal cell cancer), melanoma, and CNS tumors (e.g., a glioma, glioblastoma, astrocytoma, medulloblastoma, craniopharyrgioma, ependymoma, pinealoma, hemangioblastoma, acoustic neuroma, oligodendroglioma, meningioma, neuroblastoma, and retinoblastoma). Solid tumors also include tumor metastases (e.g., metastases to the lung, liver, brain, or bone). In particular examples, the subject has colorectal cancer or pancreatic cancer.

In some embodiments, the modified T cells are administered in combination with an agonistic CD40 antibody (for example, administered sequentially or simultaneously). In some examples, the agonistic anti-CD40 antibody may be CP-870,893 (selicrelumab), CDX-1140 (Celldex™ Therapeutics), ADC-1013 (mitazalimab), or SGN-40 (dacetuzumab). In some examples, the agonistic CD40 antibody is administered to the subject on the same day as the modified T cells. In other examples, the agonistic CD40 antibody is administered to the subject prior to the modified T cells, for example, about 1, 2, 3, or 4 weeks prior to administration of the modified T cells. In still other examples, the agonistic CD40 antibody is administered to the subject after administration of the modified T cells, for example, about 1-7 days, or about 1, 2, 3, or 4 weeks after administration of the modified T cells. In additional examples, the agonistic CD40 antibody is administered to the subject one or more times, such as once every 3-4 weeks. A skilled clinician can select timing of antibody and modified T cells administration to the subject, as well as timing and number of doses of the antibody.

In other embodiments, the modified T cells may be administered to a subject without combination with a CD40 agonist (such as without combination with an agonistic CD40 antibody). In some examples, the modified T cells express a chimeric CD40 polypeptide including a 41BB intracellular domain, a TLR4 intracellular domain, a CD28 intracellular domain, or a CD40L intracellular domain. In some examples, the modified T cells administered without combination with a CD40 agonist express CD40-4-1BBtm (such as SEQ ID NO: 100), CD40-TLR4tm (such as SEQ ID NO: 102), CD40-CD28tm_YMFM (such as SEQ ID NO: 98) or CD40-CD40Ltm (such as SEQ ID NO: 104). One of ordinary skill in the art can identify other modified T cells of this disclosure that can be administered without combination with a CD40 agonist, for example using the methods described in Example 5.

The skilled clinician will recognize that the dosages and frequencies of administration of the modified T cells and CD40 agonist (if used) can be varied, for example, as a result of clinical trials, the particular subject being treated, or other factors. In some examples, the CD40 agonist (if used) is administered to the subject prior to, substantially simultaneously with (e.g., at approximately the same time), or subsequent to administration of the modified cells. In particular examples, the T cells and/or a CD40 protein agonist is administered to the subject for up to 6 months, or in other examples for about 7-30 days. Multiple doses of the modified T cells can be administered to a subject. For example, the formulation can be administered daily, every other day, twice per week, weekly, every other week, every three weeks, monthly, or less frequently. Appropriate routes, dosages, and frequencies of administration can be determined within the discretion of a skilled clinician in a straightforward manner from readily available clinical factors, such as the medical condition and history of the subject, the tumor type being treated, and the results of clinical trials. In some embodiments, the subject is also administered at least one, at least two, at least three, or at least four cytokine(s) (e.g., IL-2, IL-15, IL-21, and IL-12) to support survival and/or growth of the modified T cells. In specific, non-limiting examples, the subject is administered IL-2. In particular examples, the cytokine(s) are administered before, after, or substantially simultaneously with the modified T cells. In some non-limiting examples, IL-2 is administered one or more times prior to or substantially simultaneously with administration of the modified cells. In particular examples, IL-2 is administered to the subject once or twice daily for 2-14 days, such as 2, 3, 4, 5, 6, 7, 8, 9, 10, 11, 12, 13, or 14 days. In other examples, the subject is administered IL-2 for about 7 days. In specific examples, at least one cytokine (e.g., IL-2) is administered simultaneously, for example, with CD8⁺CD40⁺ TCR⁺ cells.

In some examples, the subject is also treated with one or more of surgery, radiation therapy, chemotherapeutic agents, or other cancer therapies. A skilled clinician can select appropriate additional therapies for the subject, depending on factors such as the subject, the cancer being treated, treatment history, and other factors.

Exemplary chemotherapeutic agents include (but are not limited to) alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocrotical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include adriamycin, melphalan (Alkeran®) Ara-C (cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), trastuzumab (Herceptin®), irinotecan (CPT-11), leustatin, navelbine, rituximab (Rituxan®) imatinib (STI-571), Topotecan (Hycamtin®), capecitabine, ibritumomab (Zevalin®), and calcitriol.

### EXAMPLES

The following Examples are provided to illustrate certain particular features and/or embodiments. The Examples should not be construed to limit the disclosure to the particular features or embodiments exemplified.

### Example 1: Ectopic Co-Expression of CD40 and a T-Cell Receptor in T Cells

PBMC samples were thawed and set to 2e6 cells/ml in T-cell media, which consists of a 50/50 mixture of RPMI and AIM-V media supplemented with 5% in-house human serum, 10 μg/ml gentamicin, 100 U/ml penicillin and 100 μg/ml streptomycin, 1.25 μg/ml amphotericin B (Fungizone), and 2 mM L-glutamine. 2e6 cells (1 ml) were stimulated in a 24-well plate with 50 ng/ml soluble OKT3 (Miltenyi Biotec) and 300 IU/ml rhu IL-2 (Chiron) for 2 days prior to retroviral transduction.

To generate transient retroviral supernatants, the retroviral vector MSGV1 encoding wild-type CD40 and a TCR recognizing HLA-C*08:02-restricted KRASG12D neoantigen (1.5 μg/well) and the envelope-encoding plasmid RD114 (0.75 μg/well), or a control vector encoding the TCR alone and the envelope-encoding plasmid RD114 (0.75 μg/well), were co-transfected into the retroviral packaging cell line 293GP (1e6 cells per well of a 6-well poly-D-lysine-coated plates, plated the day prior to transfection) using Lipofectamine 2000 (Life Technologies). The KRASG12D neoantigen-reactive TCR contains mouse TCR constant regions to promote pairing of the introduced TCR as well as to facilitate detection of the transduced T cells. Retroviral supernatants were collected at 42-48 h after transfection, diluted 1:1 with DMEM media, and then centrifuged onto Retronectin-coated (10 μg/ml, Takara), non-tissue culture-treated 6-well plates at 2,000 g for 2 h at 32° C. Activated T cells (2e6 per well, at 0.5e6 cells/ml in 50 IU/ml IL-2 containing T-cell media) were then spun onto the retrovirus-coated plates for 10 min at 300 g. Activated T cells were transduced overnight, removed from the plates and further cultured in IL-2 containing T-cell media.

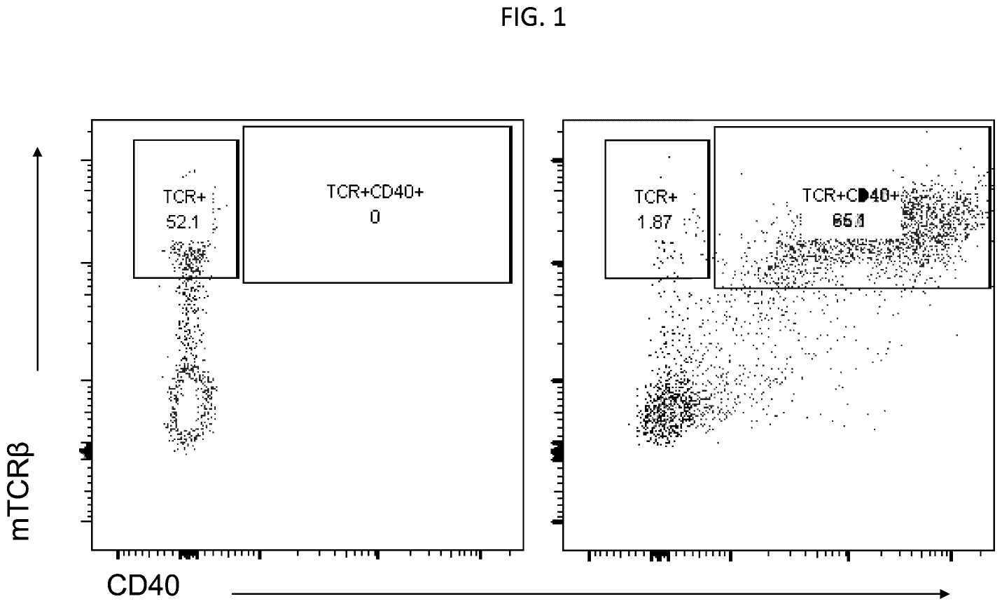

Flow cytometry was used to measure TCR transduction efficiency using a fluorochrome-conjugated anti-mouse TCRβ constant region antibody (mTCRB) and CD40 expression was measured using a fluorochrome conjugated anti-human CD40 antibody. The results showed co-expression of both CD40 and the KRASG12D neoantigen-reactive TCR in transduced CD8⁺ T cells (FIG. 1).

### Example 2: Ectopic Expression of CD40 Enhances T Cell Cytokine Secretion

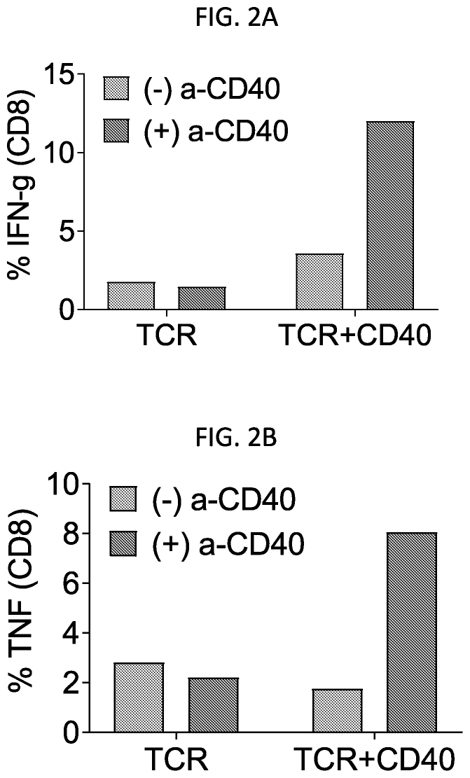

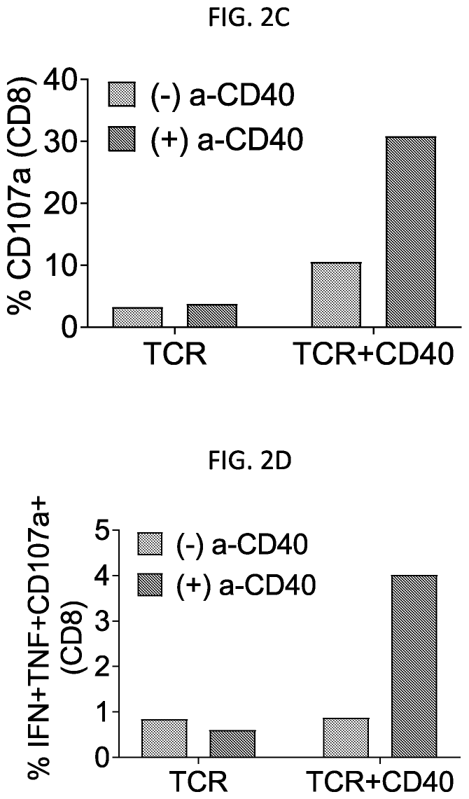

The T cells transduced to express either the HLA-C*08:02-restricted KRASG12D-reactive TCR alone, or the same TCR and CD40 were cocultured for 5 hours with a KRASG12D_positive and HLA-C*08:02-positive HPAC pancreatic cancer cell line in the presence or absence of an agonistic antibody against CD40 (CDX-1140). Intracellular cytokine staining was performed and flow cytometric analysis was used to detect the presence of IFNγ (FIG. 2A), TNF (FIG. 2B), and the degranulation marker CD107a (FIG. 2C), gated on CD8+mTCRb+ (transduced) T cells. Cells that were positive for all 3 markers (polyfunctional T cells) are shown in FIG. 2D. The ectopic expression of CD40 on T cells was observed to increase the production of effector cytokines.

### Example 3: Ectopic CD40 Expression Enhances In Vitro Tumor Cell Killing by T Cells

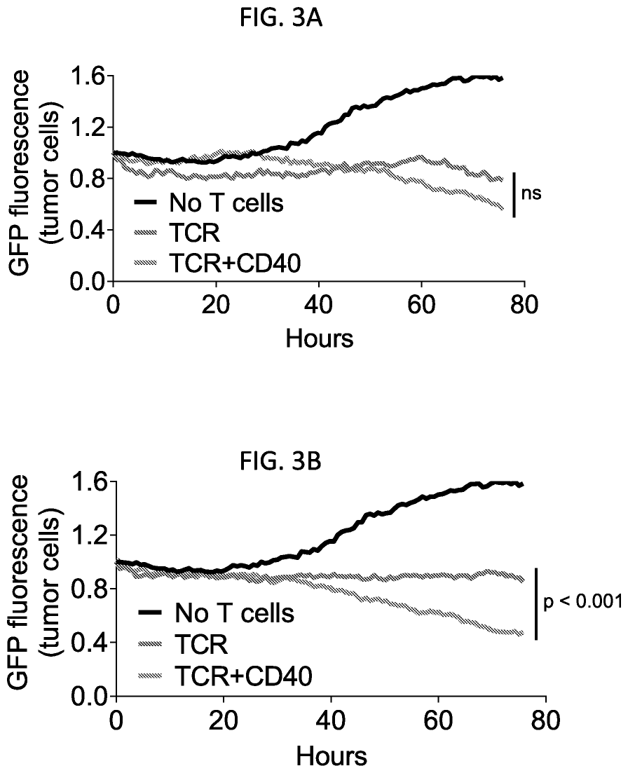

The cells described in Example 2 were tested in an in vitro tumor cell killing assay. The transduced T cells were cocultured for 3 day with the KRASG12D-positive and HLA-C*08:02-positive HPAC pancreatic cancer cell line in the presence or absence of an agonistic antibody against CD40 (CDX-1140). The HPAC cell line was modified to express GFP. Live imaging and quantification of GFP (a measure of tumor growth) was performed over the 3 days using the Incucyte live cell imager. T cells transduced to express CD40 were more effective than T cells transduced with the TCR alone at killing tumor cells in the presence of agonistic CD40 antibody (FIGS. 3A and 3B).

### Example 4: Chimeric CD40 Receptors Enhance Cytokine Secretion and Proliferation of Neoantigen-Reactive T Cells

Chimeric proteins including a CD40 extracellular domain, a transmembrane domain, and a heterologous intracellular domain were constructed. The constructs are shown in Table 1.

TABLE 1
CD40 chimeric constructs

| | Extracellular | TM | Intracellular |
|---|---|---|---|
| Name | domain | domain | domain |
| CD40tm-CD28 | CD40 | CD40 | CD28 |
| CD40-CD28tm | CD40 | CD28 | CD28 |
| CD40tm-GMCSFRa | CD40 | CD40 | GMCSFRa |
| CD40-GMCSFRatm | CD40 | GMCSFRa | GMCSFRa |
| CD40tm-CSF2Rb | CD40 | CD40 | CSF2Rb |
| CD40-CSF2Rb | CD40 | CSF2Rb | CSF2Rb |
| CD40tm-IL2Rb | CD40 | CD40 | IL2Rb |
| CD40-IL2Rbtm | CD40 | IL2Rb | IL2Rb |
| CD40tm-IL2Rg | CD40 | CD40 | IL2Rg (common |
| | | | gamma chain) |
| CD40-IL2Rgtm | CD40 | IL2Rg | IL2Rg |
| CD40-CD28tm_YMFM | CD40 | CD28 | CD28 (YMFM |
| | | | mutation) |
| CD40-IGF1Rtm | CD40 | IGF1R | IGF1R |
| CD40-4-1BBtm | CD40 | 4-1BB | 4-1BB |
| CD40-TLR4tm | CD40 | TLR4 | TLR4 |

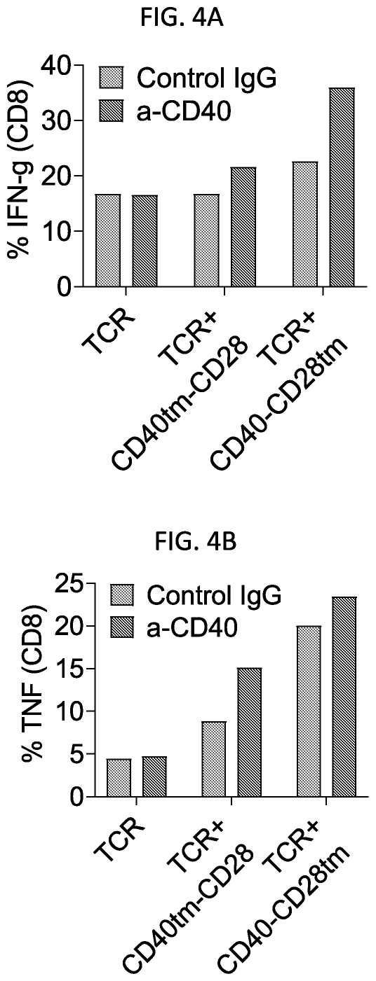

Expression of CD40-CD28 chimeric receptors enhanced cytokine production of neoantigen-reactive T cells against a cancer cell line. T cells transduced to express either the HLA-C*08:02-restricted KRAS-G12D reactive TCR alone (TCR) or the same TCR and CD40-CD28 chimeric receptors (expressing either the transmembrane domain, tm, derived from CD40 or CD28) were cocultured for 5 hours with the KRAS-G12D-positive MDA-Panc48 pancreatic cancer cell line that was transduced to express HLA-C*08:02, in the presence or absence of an agonistic antibody against CD40 (10 μg/ml, CDX-1140). Intracellular cytokine staining was performed and flow cytometric analysis was used to detect the presence of IFN-g (FIG. 4A) or TNF (FIG. 4B). Data were gated on CD8+mTCRb+ (transduced) T cells.

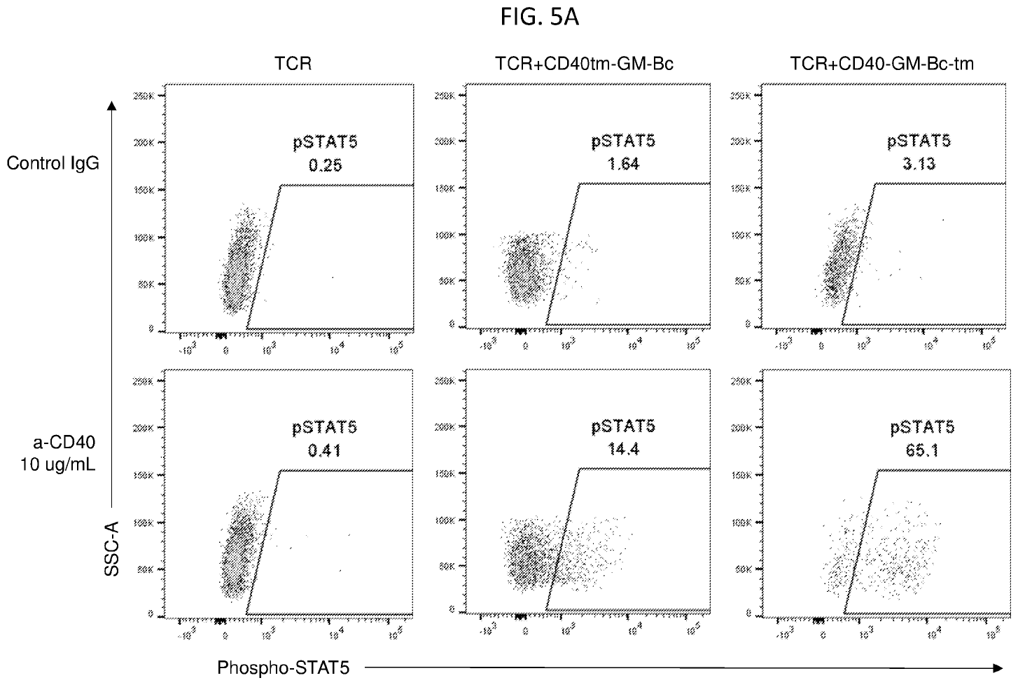

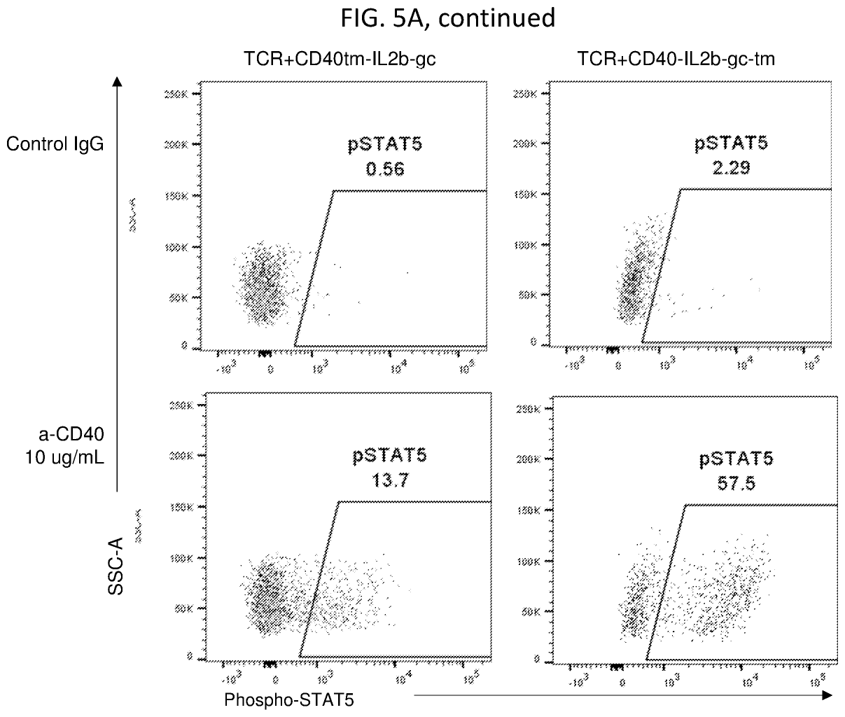

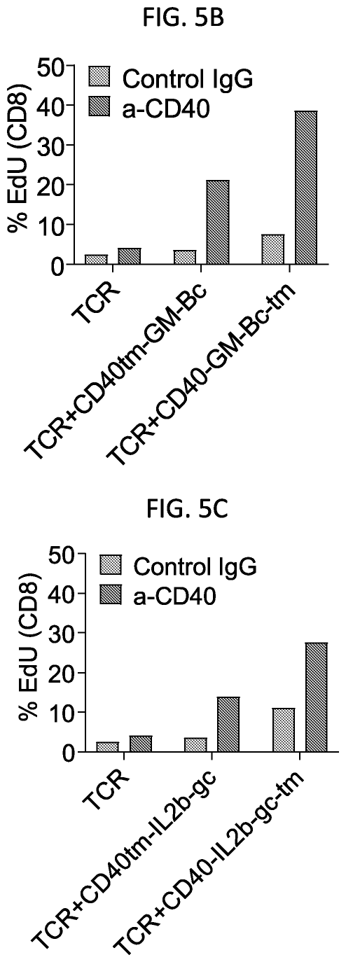

T cells expressing chimeric CD40-cytokine receptors were treated with agonistic CD40 antibodies. T cells transduced to express either the HLA-C*08:02-restricted KRAS-G12D reactive TCR alone (TCR) or the same TCR were co-transduced to express CD40-GMSCFRa (GM) and CD40-CSF2Rb (Bc), or CD40-IL2Rb (IL2b) and CD40-common gamma chain (gc) chimeric receptors (expressing either the transmembrane domain, tm, derived from CD40 or the corresponding cytokine receptors) and were stimulated for 30 minutes with or without agonistic antibody against CD40 (10 μg/ml, CDX-1140) and phospho-STAT5 was measured by intracellular flow cytometry (FIG. 5A). T cells described above were cocultured for 3 days with the KRAS-G12D-positive and HLA-C*08:02 positive HPAC cell line, in the presence or absence of an agonistic antibody against CD40 (10 μg/ml, CDX-1140) with EdU pulsed on the final day. EdU was detected by intracellular staining and flow cytometry (FIG. 5B) and (FIG. 5C). Data were gated on CD8+mTCRb+ (transduced) T cells.

### Example 5: Chimeric CD40 Receptors Enhance Tumor Cell Killing by Neoantigen-Reactive T Cells

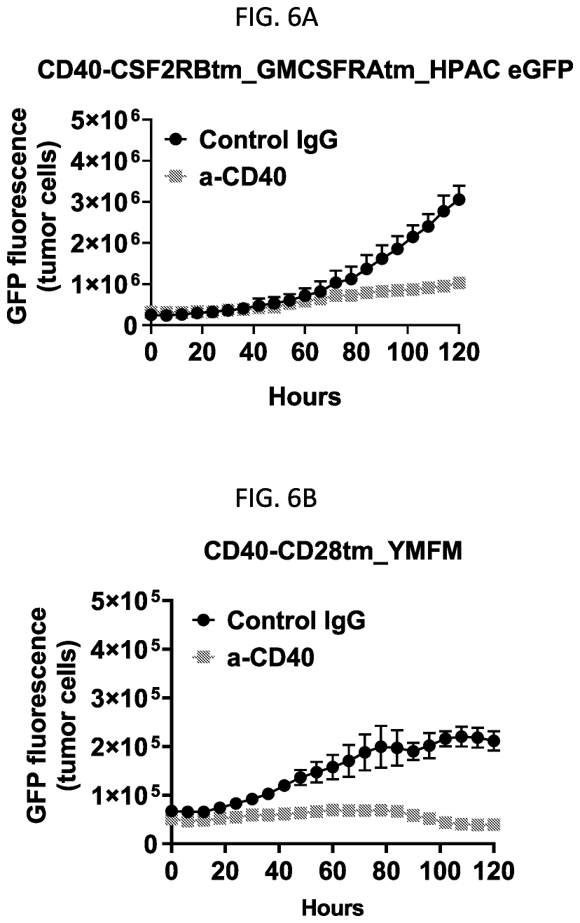

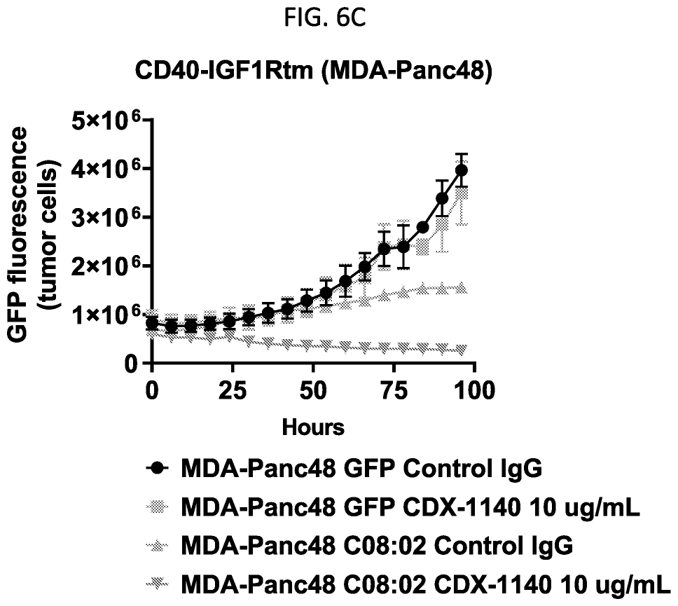

T cells co-transduced to express the HLA-C*08:02-restricted KRAS-G12D reactive TCR and CD40-CSF2Rb (Bc) (expressing the transmembrane domain, tm, derived from CSF2Rb) and GMCSFRa (GM) (expressing the transmembrane domain, tm, derived from GMCSFRa), or the same TCR and transduced CD40-CD28_YMFM chimeric receptors (expressing the transmembrane domain, tm, derived from CD28) were cocultured for 5 days with the KRAS-G12D-positive and HLA-C*08:02-positive HPAC pancreatic cancer cell line stably expressing GFP, in the absence or presence of an agonistic antibody against CD40 (10 μg/mL, CDX-1140). Live cell imaging and GFP fluorescence quantitation was measured over the 5 days using a cell imager. Addition of the agonistic anti-CD40 antibody resulted in increased tumor cell death compared to control (FIGS. 6A and 6B).

Cells expressing the same TCR and transduced to express CD40-IGF1R (expressing the transmembrane domain, tm, derived from IGF1R) chimeric receptor were cocultured for 5 days with or without the KRAS-G12D-positive and HLA-C*08:02-positive MDA-Panc48 pancreatic adenocarcinoma cell line stably expressing GFP in the absence or presence of an agonistic antibody against CD40 (10 μg/mL, CDX-1140). Live cell imaging and GFP fluorescence quantitation was measured over the 5 days using a cell imager. Tumor cell death was enhanced by addition of the agonistic anti-CD40 antibody in the presence of HLA-C*08:02-restricted KRAS-G12D reactive TCR and CD40-IGF1R (FIG. 6C).

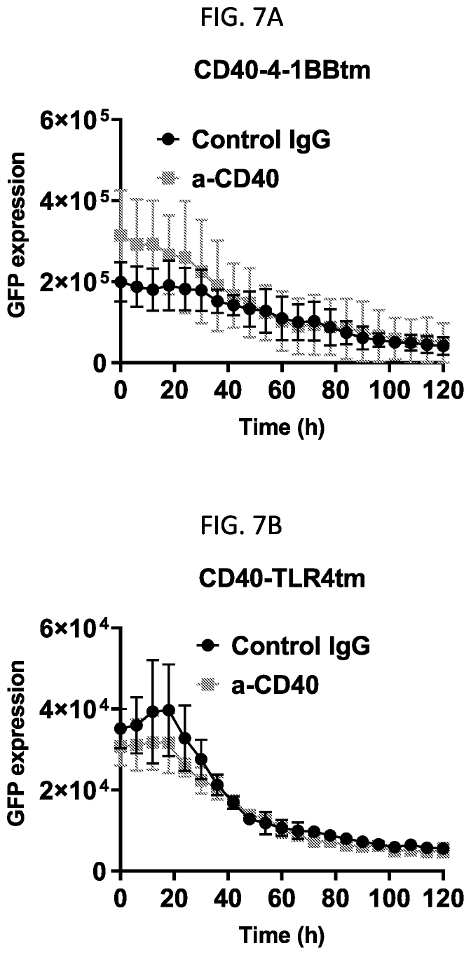

In some cases, ectopic expression of CD40 chimeric receptors enhanced in vitro tumor cell killing by neoantigen-reactive T cells in the absence of anti-CD40 antibody. T cells transduced to express the HLA-C*08:02-restricted KRAS-G12D reactive TCR and CD40-4-1BB (expressing the transmembrane domain, tm, derived from 4-1BB) (FIG. 7A) or CD40-TLR4 (expressing the transmembrane domain, tm, derived from TLR4) (FIG. 7B) were cocultured for 5 days with KRAS-G12D-positive and HLA-C*08:02-positive HPAC pancreatic cancer cell line stably expressing GFP in the absence or presence of an agonistic antibody against CD40 (10 μg/mL, CDX-1140). Live cell imaging and GFP fluorescence quantitation was measured over the 5 days using a cell imager.

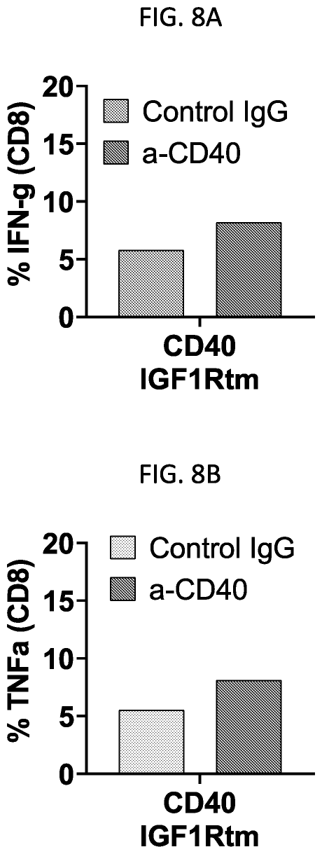

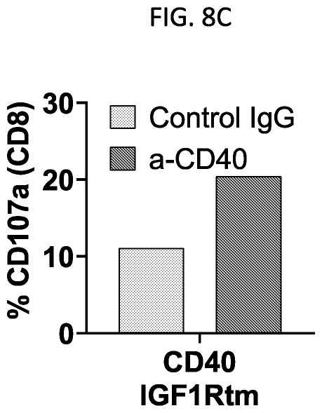

Ectopic expression of CD40 chimeric receptors also enhanced cytokine production of neoantigen-reactive T cells against a cancer cell line. T cells transduced to express the HLA-C*08:02-restricted KRAS-G12D reactive TCR and CD40-IGF1R (expressing the transmembrane domain, tm, derived from IGF1R) were cocultured for 5 hours with the KRAS-G12D-positive and HLA-C*08:02-positive HPAC pancreatic cancer cell line in the presence or absence of an agonistic antibody against CD40 (10 μg/mL, CDX-1140. Intracellular cytokine staining was performed and flow cytometric analysis was used to detect the presence of IFN-g (FIG. 8A), TNF-α (FIG. 8B), and the degranulation marker CD10⁷a (FIG. 8C). Data were gated on CD8⁺mTCRb+ (transduced) T cells.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A modified T cell comprising a nucleic acid encoding one or more chimeric polypeptide comprising a CD40 extracellular domain, a transmembrane domain, and a heterologous intracellular domain.

2. The modified T cell of claim 1, wherein the heterologous intracellular domain comprises one or more intracellular signaling domains.

3. The modified T cell of claim 1, wherein the heterologous intracellular domain is a cytokine receptor intracellular domain, a CD28 family member intracellular domain, a tumor necrosis factor receptor (TNFR) family member intracellular domain, a TNFR ligand intracellular domain, a toll-like receptor (TLR) family member intracellular domain, a receptor tyrosine kinase intracellular domain, an integrin family member intracellular domain, or a human killer immunoglobulin-like receptor (KIR) family member intracellular domain.

4. The modified T cell of claim 3, wherein:
the cytokine receptor intracellular domain is a CSF2RB, GMCSFRa, IL3Ra, IL2Rb, IL2Rg (common gamma chain), IL9R, IL7Ra, or TSLPR intracellular domain;
the CD28 family member intracellular domain is a CD28 or ICOS intracellular domain;
the TNFR family member intracellular domain is a 4-1BB, OX40, CD27, CD30, GITR, or TNFR2 intracellular domain;
the TNFR ligand intracellular domain is a RANKL, LIGHT, FasL, 41BBL, CD30L, CD27L, OX40L, TWEAK, BAFF, TRAIL, EDA, or GITRL intracellular domain;
the TLR family member intracellular domain is a TLR1, TLR2, TLR3, TLR4, TLR5, TLR6, TLR7, TLR8, TLR9, or TLR10 intracellular domain;
the receptor tyrosine kinase intracellular domain is an IGF1R, EGFR, TRKA, TRKB, TRKC, MUSK, MET, AXL, TIE2, EPHA1, EPHA3, EPHA7, EPHA8, EPHB1, EPHB2, EPHB3, EPHB6, DDR1, ROS1, LTK, or ALK intracellular domain;
the integrin family member intracellular domain is a CD11A or CD18 intracellular domain; or
the human KIR family member intracellular domain is a NKG2D, CD94, NKP46, NKP30, NKP44, NKP80, KIR2DS1, CLEC2D, or KIR3DS1 intracellular domain.

5. The modified T cell of claim 1, wherein:
the chimeric polypeptide is at least 95%, at least 97%, at least 98%, or at least 99% identical to the amino acid sequence of any one of SEQ ID NOs: 20, 24, 14, 16, 18, 22, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, or 104; or comprises the amino acid sequence of any one of SEQ ID NOs: 20, 24, 14, 16, 18, 22, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, or 104; or
the CD40 extracellular domain is at least 95%, at least 97%, at least 98%, or at least 99% identical to the amino acid sequence of SEQ ID NO: 2 or comprises the amino acid sequence of SEQ ID NO: 2.

6. The modified T cell of claim 3, wherein the heterologous intracellular domain comprises the amino acid sequence of any one of SEQ ID NOs: 5-13.

7. (canceled)

8. A modified T cell comprising a nucleic acid molecule encoding an ectopic CD40 protein.

9. The modified T cell of claim 8, wherein the ectopic CD40 protein comprises a polypeptide with at least 95% identity to the amino acid sequence of SEQ ID NO: 1 or comprises SEQ ID NO: 1.

10. The modified T cell of claim 1, wherein the CD40 protein is a human CD40 protein.

11. The modified T cell of claim 1, wherein:
the nucleic acid encoding the chimeric polypeptide or the ectopic CD40 protein is operably linked to a heterologous promoter, wherein the heterologous promoter is a constitutive promoter or an inducible promoter; or
the nucleic acid molecule encoding the chimeric polypeptide or the ectopic CD40 protein is a vector.

12-14. (canceled)

15. The modified T cell of claim 1, wherein the modified T cell is a tumor-reactive T cell, a tumor infiltrating lymphocyte (TIL), a T cell from a blood sample, or is a human T cell.

16. (canceled)

17. The modified T cell of claim 1, further comprising a heterologous T cell receptor (TCR), a chimeric antigen receptor (CAR), or both.

18-19. (canceled)

20. A pharmaceutical composition comprising a population of the modified T cells of claim 1 and a pharmaceutically acceptable carrier.

21. A method of preparing the modified T cell of claim 1, the method comprising:
transducing T cells with a nucleic acid molecule encoding the chimeric polypeptide to produce a population of transduced T cells; and
culturing the transduced T cells to produce the modified T cells.

22. The method of claim 21, further comprising transducing the T cells with a nucleic acid molecule encoding a heterologous T cell receptor, a chimeric antigen receptor, or both.

23. (canceled)

24. A method of treating a subject with cancer, comprising administering an effective amount of the modified T cell of claim 1 to the subject.

25. The method of claim 24 further comprising administering a CD40 agonist to the subject.

26-27. (canceled)

28. The method of claim 24, wherein the modified T cells are autologous to the subject.

29. (canceled)

30. A chimeric CD40 polypeptide comprising an extracellular CD40 domain, a transmembrane domain, and one or more heterologous intracellular domains.

31. The chimeric CD40 polypeptide of claim 30, wherein the extracellular CD40 domain is capable of binding CD40L or a CD40 agonistic antibody, or
wherein the transmembrane domain is a CD40 transmembrane domain or is a heterologous transmembrane domain.

32. The chimeric CD40 polypeptide of claim 30, wherein the heterologous intracellular domain is a cytokine receptor intracellular domain, a CD28 family member intracellular domain, a tumor necrosis factor receptor (TNFR) family member intracellular domain, a TNFR ligand intracellular domain, a toll-like receptor (TLR) family member intracellular domain, a receptor tyrosine kinase intracellular domain, an integrin family member intracellular domain, or a human killer immunoglobulin-like receptor (KIR) family member intracellular domain.

33. The chimeric CD40 polypeptide of claim 32, wherein:
the cytokine receptor intracellular domain is a CSF2RB, GMCSFRa, IL3Ra, IL2Rb, IL2Rg (common gamma chain), IL9R, IL7Ra, or TSLPR intracellular domain;
the CD28 family member intracellular domain is a CD28 or ICOS intracellular domain;
the TNFR family member intracellular domain is a 4-1BB, OX40, CD27, CD30, GITR, or TNFR2 intracellular domain;
the TNFR ligand intracellular domain is a RANKL, LIGHT, FasL, 41BBL, CD30L, CD27L, OX40L, TWEAK, BAFF, TRAIL, EDA, or GITRL intracellular domain;
the TLR family member intracellular domain is a TLR1, TLR2, TLR3, TLR4, TLR5, TLR6, TLR7, TLR8, TLR9, or TLR10 intracellular domain;
the receptor tyrosine kinase intracellular domain is an IGF1R, EGFR, TRKA, TRKB, TRKC, MUSK, MET, AXL, TIE2, EPHA1, EPHA3, EPHA7, EPHA8, EPHB1, EPHB2, EPHB3, EPHB6, DDR1, ROS1, LTK, or ALK intracellular domain;
the integrin family member intracellular domain is a CD11A or CD18 intracellular domain; or
the human KIR family member intracellular domain is a NKG2D, CD94, NKP46, NKP30, NKP44, NKP80, KIR2DS1, CLEC2D, or KIR3DS1 intracellular domain.

34. (canceled)

35. The chimeric CD40 polypeptide of claim 30, wherein the chimeric polypeptide is at least 95%, at least 97%, at least 98%, or at least 99% identical to the amino acid sequence of any one of SEQ ID NOs: 20, 24, 14, 16, 18, 22, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, or 104; or comprises the amino acid sequence of any one of SEQ ID NOs: 20, 24, 14, 16, 18, 22, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, or 104.

36. A nucleic acid encoding the chimeric CD40 polypeptide of claim 30.

37. The nucleic acid of claim 36, comprising a nucleic acid sequence at least 95%, at least 97%, at least 98%, or at least 99% identical to the nucleic acid sequence of any one of SEQ ID NOs: 21, 25, 15, 17, 19, 23, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, or 105; or comprises the amino acid sequence of any one of SEQ ID NOs: 21, 25, 15, 17, 19, 23, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, or 105.


SEQUENCE LISTING (105 sequences)

SEQ ID NO: 1 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 2 - protein, 193 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLR

SEQ ID NO: 3 - protein, 22 aa, synthetic construct
ALVVIPIIFGILFAILLVLVFI

SEQ ID NO: 4 - protein, 62 aa, synthetic construct
KKVAKKPTNKAPHPKQEPQEINFPDDLPGSNTAAPVQETLHGCQPVTQEDGKESRISVQERQ

SEQ ID NO: 5 - protein, 41 aa, synthetic construct
RSKRSRLLHSDYMNMTPRRPGPTRKHYQPYAPPRDFAAYRS

SEQ ID NO: 6 - protein, 42 aa, synthetic construct
KRGRKKLLYIFKQPFMRPVQTTQEEDGCSCRFPEEEEGGCEL

SEQ ID NO: 7 - protein, 42 aa, synthetic construct
ALYLLRRDQRLPPDAHKPPGGGSFRTPIQEEQADAHSTLAKI

SEQ ID NO: 8 - protein, 48 aa, synthetic construct
QRRKYRSNKGESPVEPAEPCHYSCPREEEGSTIPIQEDYRKPEPACSP

SEQ ID NO: 9 - protein, 189 aa, synthetic construct
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 10 - protein, 58 aa, synthetic construct
QLGLHIWQLRSQCMWPRETQLLLEVPPSTEDARSCQFPEEERGERSAEEKGRLGDLWV

SEQ ID NO: 11 - protein, 173 aa, synthetic construct
KKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKS

SEQ ID NO: 12 - protein, 47 aa, synthetic construct
MRRASRDYTKYLRGSEEMGGGPGAPHEGPLHAPPPPAPHQPPAASRS

SEQ ID NO: 13 - protein, 37 aa, synthetic construct
MEESVVRPSVFVVDGQTDIPFTRLGRSHRRQSCSVAR

SEQ ID NO: 14 - protein, 261 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRFWVLVVVGGVLACYSLLVTVAFIIFWVRSKRSRLLHSDYMNMTPRRPGPTRKHYQPYAPPRDFAAYRS

SEQ ID NO: 15 - DNA, 783 bp, synthetic construct
  source 1..783: mol_type = other DNA
atggtgcggctgcctctgcagtgcgtgctgtggggatgtctgctgaccgcagtgcacccagagccaccaacagcctgtagagagaagcagtacctgatcaattctcagtgctgtagcctgtgccagccaggccagaagctggtgtccgattgtaccgagttcaccgagacagagtgcctgccctgtggcgagtctgagtttctggatacctggaaccgggagacacactgccaccagcacaagtattgtgaccctaatctgggcctgagagtgcagcagaagggcacctccgagaccgacacaatctgcacatgtgaggagggatggcactgcaccagcgaggcatgcgagtcctgcgtgctgcacagatcctgttctccaggcttcggcgtgaagcagatcgccaccggcgtgtctgatacaatctgcgagccatgtcccgtgggcttctttagcaacgtgtcctctgcctttgagaagtgccacccctggaccagctgtgagacaaaggacctggtggtgcagcaggccggcaccaataagacagatgtggtgtgcggaccacaggacaggctgaggttctgggtgctggtggtggtgggaggcgtgctggcctgttactctctgctggtgaccgtggccttcatcatcttttgggtgaggtccaagcggtctagactgctgcacagcgattatatgaacatgaccccacggagacctggcccaacacgcaagcactaccagccctatgcccctccaagggactttgccgcctaccgcagc

SEQ ID NO: 16 - protein, 256 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIRSKRSRLLHSDYMNMTPRRPGPTRKHYQPYAPPRDFAAYRS

SEQ ID NO: 17 - DNA, 768 bp, synthetic construct
  source 1..768: mol_type = other DNA
atggtgcggctgcctctgcagtgcgtgctgtggggatgtctgctgaccgcagtgcacccagagccaccaacagcctgtagagagaagcagtacctgatcaattctcagtgctgtagcctgtgccagccaggccagaagctggtgtccgattgtaccgagttcaccgagacagagtgcctgccctgtggcgagtctgagtttctggatacctggaaccgggagacacactgccaccagcacaagtattgtgaccctaatctgggcctgagagtgcagcagaagggcacctccgagaccgacacaatctgcacatgtgaggagggatggcactgcaccagcgaggcatgcgagtcctgcgtgctgcacagatcctgttctccaggcttcggcgtgaagcagatcgccaccggcgtgtctgatacaatctgcgagccatgtcccgtgggcttctttagcaacgtgtcctctgcctttgagaagtgccacccctggaccagctgtgagacaaaggacctggtggtgcagcaggccggcaccaataagacagatgtggtgtgcggaccacaggacaggctgagggccctggtggtcatccccatcatcttcggcatcctgtttgccatcctgctggtgctggtgttcatcaggtccaagcggtctagactgctgcacagcgattatatgaacatgaccccacggagacctggcccaacacgcaagcactaccagccctatgcccctccaagggactttgccgcctaccgcagc

SEQ ID NO: 18 - protein, 269 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKRFLRIQRLFPPVPQIKDKLNDNHEVEDEIIWEEFTPEEGKGYREEVLTVKEIT

SEQ ID NO: 19 - DNA, 765 bp, synthetic construct
  source 1..765: mol_type = other DNA
atggtgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagagccctggtggtcatcccaatcatcttcggcatcctgtttgccatcctgctggtgctggtgttcatcaagcggttcctgagaatccagaggctgtttccccctgtgccacagatcaaggacaagctgaacgataatcacgaggtggaggacgagatcatctgggaggagttcaccccagaggagggc

SEQ ID NO: 20 - protein, 273 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRNLGSVYIYVLLIVGTLVCGIVLGFLFKRFLRIQRLFPPVPQIKDKLNDNHEVEDEIIWEEFTPEEGKGYREEVLTVKEIT

SEQ ID NO: 21 - DNA, 819 bp, synthetic construct
  source 1..819: mol_type = other DNA
atggtgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaaacctgggcagcgtgtacatctatgtgctgctgatcgtgggcaccctggtgtgcggaatcgtgctgggcttcctgtttaagcggttcctgagaatccagaggctgtttccccctgtgccacagatcaaggacaagctgaacgataatcacgaggtggaggacgagatcatctgggaggagttcaccccagaggagggcaagggctaccgggaggaggtgctgaccgtgaaggagatcaca

SEQ ID NO: 22 - protein, 652 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 23 - DNA, 1956 bp, synthetic construct
  source 1..1956: mol_type = other DNA
atggtgaggctgccactgcagtgcgtgctgtggggatgtctgctgaccgcagtgcacccagagccaccaacagcctgtcgcgagaagcagtacctgatcaattctcagtgctgtagcctgtgccagcccggccagaagctggtgtccgattgtaccgagttcaccgagacagagtgcctgccttgtggcgagtctgagtttctggatacctggaaccgggagacacactgccaccagcacaagtattgtgaccccaatctgggcctgagagtgcagcagaagggcacctccgagaccgacacaatctgcacatgtgaggagggatggcactgcaccagcgaggcatgcgagtcctgcgtgctgcaccgctcctgttctcccggcttcggcgtgaagcagatcgccaccggcgtgtctgatacaatctgcgagccctgtcctgtgggcttctttagcaacgtgtcctctgcctttgagaagtgccacccttggaccagctgtgagacaaaggacctggtggtgcagcaggccggcaccaataagacagatgtggtgtgcggacctcaggacaggctgagagccctggtggtcatcccaatcatcttcggcatcctgtttgccatcctgctggtgctggtgttcatccgcttctgcggcatctacggctatcggctgcggagaaagtgggaggagaagatcccaaacccctccaagtctcacctgtttcagaatggctctgccgagctgtggccccctggcagcatgtccgccttcacctctggcagcccaccacaccagggaccctggggctccagatttcctgagctggagggcgtgttcccagtgggatttggcgattctgaggtgagcccactgacaatcgaggaccccaagcacgtgtgcgatcctccatccggacctgacaccacaccagcagcctctgacctgccaaccgagcagccacctagccctcagccaggcccaccagcagcctcccacacaccagagaagcaggccagcagcttcgacttcaacggaccttacctgggacctccacactcccgctctctgcctgacatcctgggacagccagagccacctcaggagggaggctcccagaagtctccaccccctggcagcctggagtatctgtgcctgccagcaggaggacaggtgcagctggtgcccctggcccaggctatgggccccggccaggccgtggaggtggagaggcgcccttctcagggcgccgccggcagcccatccctggagagcggaggaggaccagcaccacccgccctgggccccagagtgggcggccaggaccagaaggattcccccgtggccatccctatgtctagcggcgacaccgaggaccccggcgtggccagcggctacgtgtcctctgccgatctggtgttcacaccaaactccggagccagctccgtgagcctggtgccttccctgggactgccatctgaccagacccccagcctgtgccccggcctggccagcggacctccaggcgcccccggccctgtgaagtctggctttgagggctatgtggagctgccacctatcgagggccggagcccaagatcccctaggaacaatcccgtgccaccagaggcaaagagccctgtgctgaatccaggcgagagaccagcagacgtgagccctacaagcccacagccagagggactgctggtgctgcagcaagtgggcgactactgcttcctgccaggcctgggccctggcccactgtccctgaggtctaagccctctagccccggccctggcccagagatcaagaacctggatcaggccttccaggtgaagaagcctccaggccaggccgtgccacaggtgcccgtgatccagctgtttaaggccctgaagcagcaggactatctgagcctgcccccttgggaagtgaataagcctggcgaggtgtgc

SEQ ID NO: 24 - protein, 647 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRVLALIVIFLTIAVLLALRFCGIYGYRLRRKWEEKIPNPSKSHLFQNGSAELWPPGSMSAFTSGSPPHQGPWGSRFPELEGVFPVGFGDSEVSPLTIEDPKHVCDPPSGPDTTPAASDLPTEQPPSPQPGPPAASHTPEKQASSFDFNGPYLGPPHSRSLPDILGQPEPPQEGGSQKSPPPGSLEYLCLPAGGQVQLVPLAQAMGPGQAVEVERRPSQGAAGSPSLESGGGPAPPALGPRVGGQDQKDSPVAIPMSSGDTEDPGVASGYVSSADLVFTPNSGASSVSLVPSLGLPSDQTPSLCPGLASGPPGAPGPVKSGFEGYVELPPIEGRSPRSPRNNPVPPEAKSPVLNPGERPADVSPTSPQPEGLLVLQQVGDYCFLPGLGPGPLSLRSKPSSPGPGPEIKNLDQAFQVKKPPGQAVPQVPVIQLFKALKQQDYLSLPPWEVNKPGEVC

SEQ ID NO: 25 - DNA, 1941 bp, synthetic construct
  source 1..1941: mol_type = other DNA
atggtgaggctgccactgcagtgcgtgctgtggggatgtctgctgaccgcagtgcacccagagccaccaacagcctgtcgcgagaagcagtacctgatcaattctcagtgctgtagcctgtgccagcccggccagaagctggtgtccgattgtaccgagttcaccgagacagagtgcctgccttgtggcgagtctgagtttctggatacctggaaccgggagacacactgccaccagcacaagtattgtgaccccaatctgggcctgagagtgcagcagaagggcacctccgagaccgacacaatctgcacatgtgaggagggatggcactgcaccagcgaggcatgcgagtcctgcgtgctgcaccgctcctgttctcccggcttcggcgtgaagcagatcgccaccggcgtgtctgatacaatctgcgagccctgtcctgtgggcttctttagcaacgtgtcctctgcctttgagaagtgccacccttggaccagctgtgagacaaaggacctggtggtgcagcaggccggcaccaataagacagatgtggtgtgcggacctcaggacaggctgagagtgctggccctgatcgtgatcttcctgaccatcgccgtgctgctggccctgcgcttctgcggcatctacggctatcggctgcggagaaagtgggaggagaagatcccaaacccctccaagtctcacctgtttcagaatggctctgccgagctgtggccccctggcagcatgtccgccttcacctctggcagcccaccacaccagggaccctggggctccagatttcctgagctggagggcgtgttcccagtgggatttggcgattctgaggtgagcccactgacaatcgaggaccccaagcacgtgtgcgatcctccatccggacctgacaccacaccagcagcctctgacctgccaaccgagcagccacctagccctcagccaggcccaccagcagcctcccacacaccagagaagcaggccagcagcttcgacttcaacggaccttacctgggacctccacactcccgctctctgcctgacatcctgggacagccagagccacctcaggagggaggctcccagaagtctccaccccctggcagcctggagtatctgtgcctgccagcaggaggacaggtgcagctggtgcccctggcccaggctatgggccccggccaggccgtggaggtggagaggcgcccttctcagggcgccgccggcagcccatccctggagagcggaggaggaccagcaccacccgccctgggccccagagtgggcggccaggaccagaaggattcccccgtggccatccctatgtctagcggcgacaccgaggaccccggcgtggccagcggctacgtgtcctctgccgatctggtgttcacaccaaactccggagccagctccgtgagcctggtgccttccctgggactgccatctgaccagacccccagcctgtgccccggcctggccagcggacctccaggcgcccccggccctgtgaagtctggctttgagggctatgtggagctgccacctatcgagggccggagcccaagatcccctaggaacaatcccgtgccaccagaggcaaagagccctgtgctgaatccaggcgagagaccagcagacgtgagccctacaagcccacagccagagggactgctggtgctgcagcaagtgggcgactactgcttcctgccaggcctgggccctggcccactgtccctgaggtctaagccctctagccccggccctggcccagagatcaagaacctggatcaggccttccaggtgaagaagcctccaggccaggccgtgccacaggtgcccgtgatccagctgtttaaggccctgaagcagcaggactatctgagcctgcccccttgggaagtgaataagcctggcgaggtgtgc

SEQ ID NO: 26 - protein, 500 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHL

SEQ ID NO: 27 - DNA, 1500 bp, synthetic construct
  source 1..1500: mol_type = other DNA
atggtgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagagccctggtggtcatcccaatcatcttcggcatcctgtttgccatcctgctggtgctggtgttcatcaactgccggaataccggcccttggctgaagaaggtgctgaagtgtaacacaccagacccctctaagttctttagccagctgagcagcgagcacggaggcgatgtgcagaagtggctgtctagcccttttccatcctctagcttcagcccaggcggcctggccccagagatctcccccctggaggtgctggagagggacaaggtgacccagctgctgctgcagcaggataaggtgcccgagcctgcctccctgtcctctaaccactccctgacctcttgctttacaaatcagggctatttctttttccacctgcctgacgccctggagatcgaggcctgtcaggtgtacttcacctatgatccctacagcgaggaggaccctgatgagggagtggcaggcgcccctacaggcagctccccacagccactgcagccactgtccggagaggacgatgcctactgcacctttccctctagggacgatctgctgctgttctctccaagcctgctgggaggaccaagcccaccttccacagccccaggcggctctggcgccggcgaggagcgcatgccacccagcctgcaggagcgggtgcccagagactgggacccccagccactgggacctccaacacccggcgtgcctgacctggtggattttcagccccctccagagctggtgctgagagaggcaggagaggaggtgcctgacgcaggaccacgggagggcgtgtcctttccctggagccggccccccggccagggagagttcagggccctgaacgcccgcctgcccctgaataccgacgcctatctgtccctgcaggagctgcagggacaggaccccacacacctg

SEQ ID NO: 28 - protein, 503 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHL

SEQ ID NO: 29 - DNA, 1509 bp, synthetic construct
  source 1..1509: mol_type = other DNA
atggtgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaatcccctggctgggacacctgctggtgggactgagcggagccttcggctttatcatcctggtgtacctgctgatcaactgccggaataccggcccttggctgaagaaggtgctgaagtgtaacacaccagacccctctaagttctttagccagctgagcagcgagcacggaggcgatgtgcagaagtggctgtctagcccttttccatcctctagcttcagcccaggcggcctggccccagagatctcccccctggaggtgctggagagggacaaggtgacccagctgctgctgcagcaggataaggtgcccgagcctgcctccctgtcctctaaccactccctgacctcttgctttacaaatcagggctatttctttttccacctgcctgacgccctggagatcgaggcctgtcaggtgtacttcacctatgatccctacagcgaggaggaccctgatgagggagtggcaggcgcccctacaggcagctccccacagccactgcagccactgtccggagaggacgatgcctactgcacctttccctctagggacgatctgctgctgttctctccaagcctgctgggaggaccaagcccaccttccacagccccaggcggctctggcgccggcgaggagcgcatgccacccagcctgcaggagcgggtgcccagagactgggacccccagccactgggacctccaacacccggcgtgcctgacctggtggattttcagccccctccagagctggtgctgagagaggcaggagaggaggtgcctgacgcaggaccacgggagggcgtgtcctttccctggagccggccccccggccagggagagttcagggccctgaacgcccgcctgcccctgaataccgacgcctatctgtccctgcaggagctgcagggacaggaccccacacacctg

SEQ ID NO: 30 - protein, 301 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 31 - DNA, 903 bp, synthetic construct
  source 1..903: mol_type = other DNA
atggtgaggctgccactgcagtgcgtgctgtggggatgtctgctgaccgcagtgcacccagagccaccaacagcctgtcgcgagaagcagtacctgatcaattctcagtgctgtagcctgtgccagcccggccagaagctggtgtccgattgtaccgagttcaccgagacagagtgcctgccttgtggcgagtctgagtttctggatacctggaaccgggagacacactgccaccagcacaagtattgtgaccccaatctgggcctgagagtgcagcagaagggcacctccgagaccgacacaatctgcacatgtgaggagggatggcactgcaccagcgaggcatgcgagtcctgcgtgctgcaccgctcctgttctcccggcttcggcgtgaagcagatcgccaccggcgtgtctgatacaatctgcgagccctgtcctgtgggcttctttagcaacgtgtcctctgcctttgagaagtgccacccttggaccagctgtgagacaaaggacctggtggtgcagcaggccggcaccaataagacagatgtggtgtgcggacctcaggacaggctgagagccctggtggtcatcccaatcatcttcggcatcctgtttgccatcctgctggtgctggtgttcatcgagcggaccatgcctagaatcccaacactgaagaacctggaggacctggtgaccgagtaccacggcaatttcagcgcctggtccggcgtgtctaagggcctggccgagagcctgcagcctgattattccgagcggctgtgcctggtgtccgagatcccacctaagggaggcgccctgggagagggccctggcgcctctccttgcaaccagcacagcccatactgggccccaccctgttataccctgaagcccgagaca

SEQ ID NO: 32 - protein, 300 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 33 - DNA, 900 bp, synthetic construct
  source 1..900: mol_type = other DNA
atggtgaggctgccactgcagtgcgtgctgtggggatgtctgctgaccgcagtgcacccagagccaccaacagcctgtcgcgagaagcagtacctgatcaattctcagtgctgtagcctgtgccagcccggccagaagctggtgtccgattgtaccgagttcaccgagacagagtgcctgccttgtggcgagtctgagtttctggatacctggaaccgggagacacactgccaccagcacaagtattgtgaccccaatctgggcctgagagtgcagcagaagggcacctccgagaccgacacaatctgcacatgtgaggagggatggcactgcaccagcgaggcatgcgagtcctgcgtgctgcaccgctcctgttctcccggcttcggcgtgaagcagatcgccaccggcgtgtctgatacaatctgcgagccctgtcctgtgggcttctttagcaacgtgtcctctgcctttgagaagtgccacccttggaccagctgtgagacaaaggacctggtggtgcagcaggccggcaccaataagacagatgtggtgtgcggacctcaggacaggctgagagtggtcatcagcgtgggcagcatgggcctgatcatctctctgctgtgcgtgtacttctggctggagcggaccatgcctagaatcccaacactgaagaacctggaggacctggtgaccgagtaccacggcaatttcagcgcctggtccggcgtgtctaagggcctggccgagagcctgcagcctgattattccgagcggctgtgcctggtgtccgagatcccacctaagggaggcgccctgggagagggccctggcgcctctccttgcaaccagcacagcccatactgggccccaccctgttataccctgaagcccgagaca

SEQ ID NO: 34 - protein, 625 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

SEQ ID NO: 35 - DNA, 1875 bp, synthetic construct
  source 1..1875: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagactgatcatcgccctgccagtggccgtgctgctgatcgtgggaggcctggtcatcatgctgtacgtgttccaccggaagagaaacaatagccggctgggaaacggcgtgctgtacgcctctgtgaatccagagtacttcagcgccgccgacgtgtacgtgcccgatgagtgggaggtggccagagagaagatcaccatgtccagagagctgggccagggctctttcggcatggtgtacgagggcgtggccaagggcgtggtgaaggatgagcccgagaccagagtggccatcaagacagtgaacgaggccgccagcatgagggagcgcatcgagttcctgaacgaggcctccgtgatgaaggagtttaattgccaccacgtggtgaggctgctgggagtggtgtcccagggacagcccaccctggtcatcatggagctgatgacacgcggcgacctgaagagctatctgcggtccctgagacctgagatggagaacaatccagtgctggcccccccttctctgagcaagatgatccagatggccggcgagatcgccgatggcatggcctacctgaacgccaataagttcgtgcaccgggacctggccgccagaaactgtatggtggccgaggacttcaccgtgaagatcggcgattttggcatgaccagggacatctatgagacagattactataggaagggaggcaagggactgctgcccgtgaggtggatgtcccctgagtctctgaaggacggcgtgttcaccacatattctgacgtgtggagctttggcgtggtgctgtgggagatcgccacactggccgagcagccataccagggcctgtccaatgagcaggtgctgcgctttgtgatggagggcggcctgctggacaagcccgataactgccctgatatgctgttcgagctgatgcggatgtgctggcagtacaatccaaagatgagaccctcttttctggagatcatcagctccatcaaggaggagatggagcctggcttccgggaggtgtccttttactattctgaggagaacaagctgccagagccagaggagctggacctggagccagagaatatggagagcgtgcccctggacccctccgcctctagctcctctctgcctctgccagacagacactctggccacaaggccgagaatggccccggccctggcgtgctggtgctgagggccagctttgacgagcgccagccttacgcccacatgaacggcggcaggaagaatgagcgcgccctgccactgccccagagcagcacctgc

SEQ ID NO: 36 - protein, 757 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIATGMVGALLLLLVVAGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 37 - DNA, 2274 bp, synthetic construct
  source 1..2274: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaatcgccaccggcatggtgggagcactgctgctgctgctggttgtagccctcggcattggactgttcatgagacggagacacatcgtgcgaaagagaacactgaggcggctgctgcaagagcgggaactggtcgagcctctgacacctagcggcgaggcccctaatcaggccctcctgagaatcctgaaggaaaccgagtttaagaagatcaaggtgctgggatctggcgcttttggtacagtgtataagggcctgtggatccccgagggcgagaaagtgaaaatccccgtggccatcaaagagctgagagaggccacatctcctaaggccaacaaggaaatcctcgatgaggcctacgtcatggccagcgttgacaacccgcacgtgtgcagactgctgggcatttgtctgactagcacagtgcagctgatcacacagctgatgcctttcggctgtctgctggactacgtgcgggaacacaaggacaacatcggcagccagtacctgcttaattggtgcgtgcaaatcgccaagggcatgaactacctggaggatagaagactggtgcacagagatctggctgctagaaacgtgctggtgaagacccctcagcacgtgaagatcacagacttcggcctggccaagctgctgggcgccgaggaaaaggagtatcacgccgaaggaggcaaggtgcctatcaagtggatggctctggaaagcatcctgcaccgaatctacacccaccagagcgacgtgtggtcctacggcgtgaccgtgtgggaactgatgaccttcggcagcaagccttacgacggcatccctgcctccgagatctcttctatcctggaaaaaggcgagcggctgcctcagcctcctatctgcaccatcgacgtgtacatgatcatggtcaaatgctggatgatcgatgccgattccagacctaagttcagagagctgattatcgagtttagcaagatggccagagaccctcagcggtacctggtgatccagggcgacgagagaatgcacctgcccagtccaaccgacagcaacttctaccgggccctgatggacgaagaggacatggatgatgtggtcgacgccgacgagtacctgatcccacagcagggatttttcagcagccctagcaccagcagaacccctctgctcagcagcctgagcgccacctccaacaatagcacagtggcctgcattgacagaaacggcctgcagtcctgccccatcaaagaggacagcttccttcagagatacagctctgatcccaccggcgccctgaccgaggattctatcgacgacaccttcctgccagttcctgagtacatcaaccagtctgtgccaaagcggcccgcaggcagcgtgcagaaccccgtgtaccataaccagcccctgaaccctgctcctagccgcgaccctcactaccaagatcctcacagcaccgccgtgggcaatcctgaatatctgaacaccgtgcagcctacctgtgtgaacagcacattcgacagccccgctcattgggcccagaagggcagccaccagatcagcctggacaatcctgattaccagcaggacttcttccccaaggaagctaagcccaacggcatcttcaaaggaagcaccgccgagaacgccgagtacctgagagtggccccacaatcttctgagttcatcggagcc

SEQ ID NO: 38 - protein, 566 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLAVFACLFLSTLLLVLNKCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLG

SEQ ID NO: 39 - DNA, 1698 bp, synthetic construct
  source 1..1698: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagactcgccgtgttcgcctgtctgttcctttctaccctgctgctcgtgctgaacaagtgcggcaggcggaacaaattcggcatcaacagacccgccgtgctggcacctgaagatggcctggccatgagcctgcactttatgacactgggcggatctagcctgtctcctacagaaggcaagggcagcggactgcagggccacatcatcgagaatcctcaatacttcagcgacgcctgtgtgcaccacattaagcggagagacatcgtgctgaaatgggaactgggcgaaggcgccttcggaaaggtgttcctggccgagtgccacaacctgctgcctgagcaggataagatgctggtggccgtgaaagccctgaaggaagctagcgagtccgccagacaggactttcagcgggaagccgaactgctgacaatgctgcagcaccagcacatcgtgcggttctttggtgtttgtaccgaaggcagacctctgctgatggtctttgagtacatgcggcatggcgatctgaatcggttcctgaggagccacggccctgatgccaagctgctggctggcggagaggacgtggctcccggccccctgggcctgggacaactcctggctgttgccagccaggtggccgctggcatggtgtacctggccggcctgcacttcgtgcatagagacctggccaccagaaactgcctggtgggccagggcctggtcgtgaagatcggcgacttcggcatgagcagagacatctacagcacagactactacagagtgggcggccgcaccatgctgccaatcagatggatgcctccagagagcatcctgtaccggaagttcaccaccgagtctgatgtgtggtccttcggcgtggtgctgtgggagatcttcacctacggcaagcagccttggtatcagctgagcaacaccgaggccatcgactgcatcacccagggcagagagctggaaagaccgcgggcctgcccccccgaggtgtacgccatcatgagaggctgctggcagagagagcctcagcagagacacagcatcaaggacgtgcacgccagattgcaagctctggcccaggcccctcctgtgtatctggacgtgctggga

SEQ ID NO: 40 - protein, 585 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLSVYAVVVIASVVGFCLLVMLFLLKLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 41 - DNA, 1755 bp, synthetic construct
  source 1..1755: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagactctccgtgtacgccgtagtcgtgatcgcctccgtggttggattttgcctgctggtgatgctgttcctgcttaagctggctcggcacagcaagttcggaatgaaaggccctgctagcgtgatttctaacgacgatgacagcgcctctcctctgcaccacatcagcaacggcagcaataccccttctagcagcgagggcggccctgatgctgtgattatcggtatgaccaagatccctgtgatcgaaaatcctcagtacttcggcatcacaaacagccaactgaagcccgacaccttcgtgcagcacatcaagagacacaatatcgtgctgaaacgggaactgggcgagggcgccttcggcaaagtgttcctggccgaatgctacaacctgtgccctgaacaggacaagatcctggtggccgtgaagaccctgaaggacgcctctgacaacgccagaaaggacttccaccgggaagccgagctgcttacaaacctgcagcacgagcacatcgtgaaattttacggcgtgtgcgtggaaggcgaccccctgatcatggtgttcgagtacatgaagcacggcgacctgaacaaattcctgagagcccacggccctgatgccgttctgatggccgagggcaaccctcccaccgagctgacccagagccagatgctgcatatcgcccagcagatcgccgccggcatggtgtacctggccagccagcatttcgtccaccgcgacctggccaccagaaactgcctggtgggagagaacctgctggtgaagatcggcgatttcggcatgagcagagatgtgtacagcaccgactactacagagtgggcggccacaccatgctgcctatcagatggatgcctccagaaagcatcatgtaccggaagtttacaaccgagtctgatgtgtggtccctgggagtggtcctgtgggagatcttcacatatggaaagcagccttggtaccagctgagcaacaatgaggtgattgagtgtatcacacagggcagagtgctgcaaagacccagaacctgcccccaagaggtgtacgagctgatgctgggatgttggcagcgggaaccccacatgaggaagaacatcaagggcatccacacactgctgcagaacctcgctaaggcttctccagtgtatctggacatcctgggc

SEQ ID NO: 42 - protein, 603 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRFGVSIAVGLAAFACVLLVVLFVMINKYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRLFNPSGNDFCIWCEVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 43 - DNA, 1809 bp, synthetic construct
  source 1..1809: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagattcggagtcagcatcgccgtgggcctggctgcttttgcctgcgtgctgctcgtggtcctgttcgtgatgatcaacaaatacggcagaaggtccaagttcggcatgaaaggcccagtggccgtgatcagcggcgaggaagatagcgcctctcctctgcatcacattaaccacggcatcacaacaccttccagcctggacgctggccctgacacagtggtgatcggcatgacaagaatccctgtgatcgagaaccctcagtacttccggcaaggccacaactgccacaagcctgatacatacgtgcagcatatcaagcggcgggacatcgtgctgaagagagagctgggagagggcgccttcggcaaagtgttcttggccgagtgctacaacctgagccctaccaaggataagatgctggtcgccgtgaaggccctgaaagaccccaccctggccgctagaaaggacttccagcgggaagccgagctgctgaccaacctgcagcacgagcacatcgtgaaattctacggagtgtgtggcgacggcgatcccctgatcatggtttttgagtacatgaagcacggcgacctgaacaagttcctgagagcccacggcccagacgccatgatcctggtggacggccagcctagacaggctaagggcgaactgggcctctcccaaatgctgcacatcgccagccagatcgccagcggaatggtgtatctggcctctcagcacttcgtgcacagagatctggcaaccagaaactgcctggtgggagccaacctgctggtgaagatcggggacttcggcatgagcagagatgtgtatagcaccgactactaccggctgtttaatcctagcggcaacgacttctgcatttggtgcgaggtgggaggccacacaatgctccccatccggtggatgcctcctgagagcatcatgtaccgcaagttcaccaccgaaagcgacgtatggtcttttggcgtgatcctgtgggaaatcttcacctacggcaagcagccctggttccagctgtctaataccgaggtgatcgagtgtatcacccaggggagagtgctggaaagacccagagtgtgccccaaggaagtgtacgacgtgatgctgggatgttggcagagagagccccagcagcggctgaatatcaaggaaatttacaagatcctgcacgccctgggcaaggccacccctatctacctggatatcctgggc

SEQ ID NO: 44 - protein, 567 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRVIISIMSSFAIFVLLTITTLYCCRRRKQWKNKKRESAAVTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEFLRSMSPHTVCSLSHSDLSMRAQVSSPGPPPLSCAEQLCIARQVAAGMAYLSERKFVHRDLATRNCLVGENMVVKIADFGLSRNIYSADYYKANENDAIPIRWMPPESIFYNRYTTESDVWAYGVVLWEIFSYGLQPYYGMAHEEVIYYVRDGNILSCPENCPVELYNLMRLCWSKLPADRPSFTSIHRILERMCERAEGTVSV

SEQ ID NO: 45 - DNA, 1701 bp, synthetic construct
  source 1..1701: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagagtcatcatcagcatcatgtcttcttttgccatcttcgtgctgctcaccatcacaacactgtactgctgcagacggagaaagcagtggaagaacaagaaaagagaatctgctgctgttacactgaccaccctgccaagcgagctgctgcttgatagactgcatcctaatcccatgtaccagagaatgcccctgctcctcaaccctaagctgctgagcctggaataccccagaaacaacatcgagtacgtgcgggacatcggcgagggcgccttcggcagagtgttccaggcccgggctcctggactgctgccttacgagcctttcacaatggtggccgtgaagatgctgaaggaagaggccagcgccgatatgcaggctgacttccaaagagaggccgccctgatggccgagttcgacaaccccaacattgtgaaactgttgggcgtgtgcgccgtgggaaaacctatgtgtctgctgttcgagtacatggcctacggcgatctgaatgagttcctgaggtccatgagcccccacaccgtgtgttctctgagccacagcgacctgtccatgagagcccaggtgtccagccctggccctccaccactgtcctgcgccgaacagctgtgcatcgctcgccaggtggccgctggcatggcttacctgtctgaacggaagttcgtgcacagggatctggccaccagaaattgtctggtgggcgaaaacatggtggtcaagatcgccgatttcggcctgagcagaaacatctacagcgccgactactacaaggccaacgagaacgacgccatccccatccggtggatgcctcctgagagcatcttttacaatagatacaccacagagagcgacgtgtgggcctatggagtggtgctgtgggagatctttagctacggcctgcagccttactacggaatggcccacgaggaagtgatttattatgtgagagacggcaacatcctgagctgtcctgagaactgccccgtggaactgtacaacctgatgcggctgtgctggagcaagctgcctgccgacagaccttctttcacaagcatccaccggatcctggaaagaatgtgcgagcgggccgagggcaccgtgagcgtg

SEQ ID NO: 46 - protein, 651 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 47 - DNA, 1953 bp, synthetic construct
  source 1..1953: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaggcctgatcgccggcgtggtatctatcagcacagccctgctgctcctcctgggcttcttcctgtggctgaaaaagcggaagcagatcaaggacctgggaagcgagctggtcagatatgacgccagagtgcacacccctcacctggatagactggtgtctgccagaagcgtctctcctaccacagagatggtgtccaacgagtccgtggactaccgggccacattcccagaagatcagtttcctaacagcagccagaacggctcttgtagacaggtgcaataccctctgaccgacatgagccctatcctgacaagcggcgacagcgatatcagcagccccctactgcagaacaccgttcatatcgacctgtctgccctgaaccccgaactggtgcaggccgtgcagcacgtggtgatcggtccaagctctcttattgtgcactttaacgaggtcatcggaagaggccacttcggctgcgtgtaccacggcaccctgctggacaacgacggcaagaagatccactgcgccgtgaaaagcctgaatagaatcacagatatcggcgaggtgtcccagttcctgaccgagggcatcatcatgaaagacttcagccaccctaatgtgctgagcctgctgggcatttgccttcggagcgagggcagccctctggtcgtgctgccttacatgaagcacggcgatctgagaaacttcatccggaatgaaacccacaatcctacagtgaaggatctgatcggatttggcctgcaggttgcaaagggcatgaaatacctggcttctaagaaattcgtgcatagagatctggccgctagaaactgcatgctggacgagaagtttaccgtgaaggtggccgatttcggactggcccgggacatgtacgacaaggaatactacagcgtgcacaacaagaccggcgctaagctgcccgtgaagtggatggccttggaatccctgcaaacacagaaattcaccactaagtccgatgtgtggtccttcggcgtgctgctgtgggagctgatgacccggggcgctcctccttatccagacgtgaacacattcgacatcaccgtgtacctgctgcagggcaggcggctgctgcagcctgagtactgccccgaccccctgtacgaggtgatgctgaagtgctggcaccccaaggccgagatgagacctagcttcagcgaactggtgagtagaatcagcgctatcttcagcaccttcatcggagaacactacgtgcacgtgaacgccacctacgtgaacgtgaagtgtgtggccccttaccccagcctgctgagcagcgaggataacgccgacgacgaggtggacaccagacccgccagcttctgggaaaccagc

SEQ ID NO: 48 - protein, 636 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 49 - DNA, 1908 bp, synthetic construct
  source 1..1908: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagatatgtgctgctcggcgccgtggtggccgccgcttgtgtgctgatcctggccctgttcctggttcatagacggaagaaggaaacccggtacggcgaggtgttcgagcccaccgtggaaagaggagaactggtggtcagataccgggtcagaaagagctacagcagaaggaccaccgaggccacactgaacagcctgggcatcagcgaggaacttaaagaaaagctgagagatgtgatggtggaccggcacaaggtggccctgggcaagaccctgggggaaggcgagtttggcgcagtgatggaaggccaactgaaccaggatgatagcatcctgaaggtggccgtcaagaccatgaagatcgctatctgcacccgcagcgagctcgaggacttcctgagcgaggccgtttgcatgaaagagttcgaccaccccaacgtgatgcggctgatcggcgtgtgcttccagggctctgaaagagagagcttccccgcccctgtggtgatcctgcctttcatgaagcacggcgatctgcactcctttctgctgtacagcagactgggcgaccagcctgtgtacctgcccacacagatgctggtgaagttcatggccgacatcgccagcggcatggaatacctgagcaccaagcggttcattcaccgggacctggccgccagaaactgcatgctgaatgagaacatgagcgtgtgcgtggccgattttggcctgtctaaaaagatctacaacggcgactactacagacagggcagaatcgccaagatgcctgtgaagtggatcgccatcgagtctctggctgatagagtgtacaccagcaagagcgacgtgtggtccttcggcgtgaccatgtgggagattgccacacggggccaaacaccatatcccggagtggaaaatagcgagatctacgactacctgagacagggaaatagactgaaacagcctgctgactgcctggacggcctgtacgccctgatgagccggtgctgggaactgaacccccaggacagaccttctttcaccgagctgagagaagatctggaaaacacactgaaagccctgccaccagcccaggagcctgacgagatcctgtatgtgaacatggacgagggcggaggctacccggaacctccaggagccgccggcggcgccgaccctcctacccagcctgatcctaaggactcctgctcttgtctgacagccgccgaggtgcaccctgccggaagatacgtgctgtgtccttccacaacccctagccctgctcagcccgccgatcggggcagccccgctgctcctggccaggaggacggcgcc

SEQ ID NO: 50 - protein, 569 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIAILGSAGMTCLTVLLAFLIILQLKRANVQRRMAQAFQNVREEPAVQFNSGTLALNRKVKNNPDPTIYPVLDWNDIKFQDVIGEGNFGQVLKARIKKDGLRMDAAIKRMKEYASKDDHRDFAGELEVLCKLGHHPNIINLLGACEHRGYLYLAIEYAPHGNLLDFLRKSRVLETDPAFAIANSTASTLSSQQLLHFAADVARGMDYLSQKQFIHRDLAARNILVGENYVAKIADFGLSRGQEVYVKKTMGRLPVRWMAIESLNYSVYTTNSDVWSYGVLLWEIVSLGGTPYCGMTCAELYEKLPQGYRLEKPLNCDDEVYDLMRQCWREKPYERPSFAQILVSLNRMLEERKTYVNTTLYEKFTYAGIDCSAEEAA

SEQ ID NO: 51 - DNA, 1707 bp, synthetic construct
  source 1..1707: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaatcgccatcctgggcagcgctggcatgacatgcctgaccgtgctcctggctttcctgatcatcctgcagctgaagagagccaatgtgcagagaagaatggcccaggcctttcagaacgtgcgggaagagcctgccgtgcagtttaacagcggcacactggccctgaacagaaaggtgaaaaacaaccctgatcccaccatctaccctgtgctggattggaacgacatcaagttccaggacgttatcggcgaaggcaacttcggccaggtgctgaaggccagaatcaagaaggacggcctgagaatggatgccgctattaagcggatgaaggaatacgcctctaaggatgaccacagagacttcgccggcgagctggaagtgctttgtaaactgggccaccatcctaatatcatcaacctgctcggcgcttgtgaacacagaggctatctgtacctggctatcgagtacgcccctcacggaaatctgctggacttcctgcggaaaagcagagtgcttgagacagaccccgccttcgcaatcgccaacagcaccgcctctaccctgtccagccaacagctgctgcacttcgctgccgacgtggctagaggcatggactacctgagccagaaacagttcatccaccgggatctggccgcccgcaacatcctggtcggagaaaactacgtcgccaaaatcgccgatttcggactgagccggggacaggaggtctacgtgaagaagacaatgggcaggctgccagtgcggtggatggccattgagagcctgaactactccgtgtatacaacaaacagcgatgtgtggagctacggcgtgctgctgtgggagatcgtgtctctgggcgggaccccttactgcggcatgacctgcgccgagctgtatgagaagctgccccagggctacagactggaaaagcccctgaactgcgacgacgaggtgtacgacctgatgcggcagtgctggagagagaagccttacgagcgtccatcttttgcccaaatcctggtgtccctgaatagaatgctggaagaaagaaagacctacgtgaacaccacactgtacgagaagttcacctacgccggcatcgactgcagcgccgaggaggccgcc

SEQ ID NO: 52 - protein, 622 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIVAVIFGLLLGAALLLGILVFRSRRAQRQRQQRQRDRATDVDREDKLWLKPYVDLQAYEDPAQGALDFTRELDPAWLMVDTVIGEGEFGEVYRGTLRLPSQDCKTVAIKTLKDTSPGGQWWNFLREATIMGQFSHPHILHLEGVVTKRKPIMIITEFMENGALDAFLREREDQLVPGQLVAMLQGIASGMNYLSNHNYVHRDLAARNILVNQNLCCKVSDFGLTRLLDDFDGTYETQGGKIPIRWTAPEAIAHRIFTTASDVWSFGIVMWEVLSFGDKPYGEMSNQEVMKSIEDGYRLPPPVDCPAPLYELMKNCWAYDRARRPHFQKLQAHLEQLLANPHSLRTIANFDPRMTLRLPSLSGSDGIPYRTVSEWLESIRMKRYILHFHSAGLDTMECVLELTAEDLTQMGITLPGHQKRILCSIQGFKD

SEQ ID NO: 53 - DNA, 1866 bp, synthetic construct
  source 1..1866: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaattgtggccgtgatctttggactgctgctgggagctgctctgctgctgggcatcctggtgttcagatctagaagagcccagaggcagcggcagcagaggcagcgggacagagcaaccgacgtggaccgggaagataagctgtggctgaaaccttacgtggacctgcaagcttatgaggatccagctcagggcgccctggatttcaccagagaactggatcccgcctggctgatggtggacacagttatcggcgagggagagttcggcgaggtgtacagaggcaccctgcggctgcccagccaggactgcaagacagtggccatcaagaccctgaaggacaccagccctggcggacagtggtggaatttcctgcgggaagccaccatcatgggccaatttagccacccccacatcctgcacctggaaggcgtggtgaccaagagaaagcctatcatgatcatcaccgagttcatggaaaacggcgccctggacgccttcctgagagagagagaggaccagctggtgccaggacagctggttgctatgctgcaaggtatcgcctccggcatgaactacctgagcaatcacaactacgtgcacagagatctggccgccagaaacatcctggtcaaccagaacctgtgctgtaaagtgagcgacttcggactgaccagattgctcgacgacttcgatggcacatacgaaacccagggcggcaagatccccatccggtggacagcccctgaggccatcgcccaccggatctttacaaccgccagcgacgtgtggtccttcggcatcgtgatgtgggaggtgctgtctttcggcgataagccctacggcgagatgagcaaccaggaggtcatgaagagcatcgaggatggctaccggcttcctccacctgtggattgccctgctcctctgtacgagctgatgaaaaactgctgggcctacgacagagccagacggcctcactttcagaaactgcaggcccatctggaacagctgctggccaatcctcattctctgcgtaccatcgccaacttcgaccccagaatgaccctgagactgcctagcctgagcggctctgacggcatcccttatagaacagtgtccgagtggttggaaagcatcagaatgaagcggtacatcctgcacttccacagcgccggcctggatacaatggagtgcgtgctggagctgacagccgaggacctgacacaaatgggcattaccctgcctggccaccagaagcggatcctgtgtagcatccagggtttcaaggac

SEQ ID NO: 54 - protein, 635 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRVVMIAISAAVAIILLTVVIYVLIGRFCGYKSKHGADEKRLHFGNGHLKLPGLRTYVDPHTYEDPTQAVHEFAKELDATNISIDKVVGAGEFGEVCSGRLKLPSKKEISVAIKTLKVGYTEKQRRDFLGEASIMGQFDHPNIIRLEGVVTKSKPVMIVTEYMENGSLDSFLRKHDAQFTVIQLVGMLRGIASGMKYLSDMGYVHRDLAARNILINSNLVCKVSDFGLSRVLEDDPEAAYTTRGGKIPIRWTSPEAIAYRKFTSASDVWSYGIVLWEVMSYGERPYWEMSNQDVIKAVDEGYRLPPPMDCPAALYQLMLDCWQKDRNNRPKFEQIVSILDKLIRNPGSLKIITSAAARPSNLLLDQSNVDITTFRTTGDWLNGVWTAHCKEIFTGVEYSSCDTIAKISTDDMKKVGVTVVGPQKKIISSIKALETQSKNGPVPV

SEQ ID NO: 55 - DNA, 1905 bp, synthetic construct
  source 1..1905: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagagtggtgatgatcgccatctccgccgccgtggccatcattctgctgaccgtggttatctacgtgcttatcggcagattctgcggctataagagcaagcacggcgccgacgagaagcggctgcacttcggcaacggccacctgaagctgcccggcctgaggacctacgtggatccccacacctacgaagaccctacacaggccgtgcacgagttcgccaaggaactggacgccacaaacatcagcatcgacaaggtggtgggagctggcgagtttggcgaagtgtgcagcggaagactgaagctgcctagcaagaaggaaatcagcgtggccattaagaccctgaaggtgggatatacagagaagcaacgtagagattttctgggcgaggcctccatcatgggacagttcgaccaccctaacatcatccggctggaaggcgtcgtgaccaagtctaagcccgtgatgatcgtgaccgagtacatggaaaacggcagcctggacagcttcctgagaaaacatgacgcccagttcaccgtgatccagctggtgggaatgctgcggggaatcgcctctggcatgaaatacctgtctgacatgggctacgtgcacagagacctggcggctagaaacatcctgattaacagcaacctggtctgcaaggtgtccgacttcggcctgagcagagtgctggaagatgatcctgaagctgcttacaccaccagaggcggcaagatccccatccggtggacctcccctgaggccatcgcctacagaaagttcaccagcgcttctgatgtgtggtcctacggaatcgtgctgtgggaggtgatgtcctacggcgagcggccttactgggagatgagcaaccaggacgtgatcaaggccgttgacgagggctaccggctgcctccacctatggactgtcctgccgccctgtaccagctgatgctggactgctggcagaaagacagaaacaaccgccccaagttcgagcagatcgtcagcatcctcgataagctgatcagaaatcctggcagcctgaaaatcatcaccagcgccgccgccagaccctctaatctgctgctggatcagagcaatgtggacatcacaacttttagaaccacaggagattggctgaacggcgtttggacagcccactgtaaagagatcttcacaggcgtggaatacagctcatgcgacaccatcgctaagatcagcaccgatgacatgaaaaaggtgggcgtgacagtggtgggcccacagaagaaaattatcagcagcatcaaggccctcgagacacaatctaagaacggccctgtgcctgtg

SEQ ID NO: 56 - protein, 636 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIIIAVVAVAGTIILVFMVFGFIIGRRHCGYSKADQEGDEELYFHFKFPGTKTYIDPETYEDPNRAVHQFAKELDASCIKIERVIGAGEFGEVCSGRLKLPGKRDVAVAIKTLKVGYTEKQRRDFLCEASIMGQFDHPNVVHLEGVVTRGKPVMIVIEFMENGALDAFLRKHDGQFTVIQLVGMLRGIAAGMRYLADMGYVHRDLAARNILVNSNLVCKVSDFGLSRVIEDDPEAVYTTTGGKIPVRWTAPEAIQYRKFTSASDVWSYGIVMWEVMSYGERPYWDMSNQDVIKAIEEGYRLPAPMDCPAGLHQLMLDCWQKERAERPKFEQIVGILDKMIRNPNSLKTPLGTCSRPISPLLDQNTPDFTTFCSVGEWLQAIKMERYKDNFTAAGYNSLESVARMTIEDVMSLGITLVGHQKKIMSSIQTMRAQMLHLHGTGIQV

SEQ ID NO: 57 - DNA, 1908 bp, synthetic construct
  source 1..1908: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaattatcatcgccgtggtggccgtggccggcacaatcatcctggttttcatggtgttcggcttcatcattggcagacggcactgcggctacagcaaggccgatcaggagggagatgaggaactgtacttccacttcaagtttccaggcaccaagacctacatcgaccctgagacatacgaggaccccaatagagccgtgcaccagtttgccaaggaactggatgccagctgcatcaagatcgagcgggtgatcggcgctggagagttcggcgaggtgtgtagcggcaggctgaagctgcctggcaaaagagatgtggctgtggccattaagaccctgaaagtgggatatacagagaagcagagaagagactttctgtgcgaggcatctatcatgggccagttcgaccaccctaatgtggtgcacctggaaggcgtggtcaccagaggcaagcctgtgatgatcgtaattgagttcatggaaaacggcgccctggacgccttcctgagaaagcacgacggccagttcaccgtgatccagctggtcgggatgctgagaggaatcgccgctggcatgcggtacctggccgacatgggctacgtgcacagagatctggctgctagaaacatcctggtgaactctaacttggtgtgcaaggtgtccgactttggactgagcagagtgatcgaggacgaccctgaggccgtgtacaccaccaccggtggaaaaatccccgtcaggtggaccgcccctgaagctatccagtacagaaagttcacgagcgcctctgacgtttggtcctatggcatcgtgatgtgggaggtgatgtcctacggcgagcggccctactgggacatgagcaaccaggatgtgatcaaggccatcgaggaaggctaccggctgcccgctccaatggactgtcctgccggactgcaccaactgatgctggattgctggcagaaggaacgggccgagagacccaagttcgagcagatcgtgggcatcctggacaagatgatccggaaccctaatagcctgaagacacctctgggcacctgtagccggccaatcagccctctgctggaccagaacacccctgacttcaccacattctgcagcgtgggcgaatggctccaggccatcaaaatggaaagatacaaggacaacttcacagccgccggctacaacagcctggaaagcgtggcccgcatgaccatcgaggatgtgatgagcctgggcatcaccctggtgggccaccagaaaaagatcatgtctagcatccaaacaatgagagcccagatgcttcatctgcacggcaccggcatccaagtg

SEQ ID NO: 58 - protein, 656 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIVWICLTLITGLVVLLLLLICKKRHCGYSKAFQDSDEEKMHYQNGQAPPPVFLPLHHPPGKLPEPQFYAEPHTYEEPGRAGRSFTREIEASRIHIEKIIGSGDSGEVCYGRLRVPGQRDVPVAIKALKAGYTERQRRDFLSEASIMGQFDHPNIIRLEGVVTRGRLAMIVTEYMENGSLDTFLRTHDGQFTIMQLVGMLRGVGAGMRYLSDLGYVHRDLAARNVLVDSNLVCKVSDFGLSRVLEDDPDAAYTTTGGKIPIRWTAPEAIAFRTFSSASDVWSFGVVMWEVLAYGERPYWNMTNRDVISSVEEGYRLPAPMGCPHALHQLMLDCWHKDRAQRPRFSQIVSVLDALIRSPESLRATATVSRCPPPAFVRSCFDLRGGSGGGGGLTVGDWLDSIRMGRYRDHFAAGGYSSLGMVLRMNAQDVRALGITLMGHQKKILGSIQTMRAQLTSTQGPRRHL

SEQ ID NO: 59 - DNA, 1968 bp, synthetic construct
  source 1..1968: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaatcgtgtggatctgtctgaccctgatcacaggcctggtggtgctgctgctgctgcttatttgcaagaagcggcactgcggctacagcaaggcctttcaagactctgacgaggaaaagatgcactaccagaacggccaggcccctccccctgttttcctgccactgcaccaccctcctgggaaactgcccgagccccagttctacgccgaacctcacacctacgaggagcccggcagagccggcagaagcttcacccgggaaatcgaggccagcagaatccacatcgagaagatcatcggctctggtgattctggcgaggtgtgctacggccggctgcgggtgccaggccaaagagacgtgcctgtggccatcaaggccctgaaggccggatataccgagcggcagagacgggacttcctgagcgaggccagcatcatgggccagtttgaccaccccaatatcatcagactggaaggcgtggtgaccagaggaagactggccatgatcgtgacagagtacatggaaaacggcagcctggacaccttcctgcggacccacgacggccagttcacaatcatgcagctggtgggaatgctgagaggcgttggagccggcatgcggtacctgagcgatctgggctacgtgcacagagatctggctgctagaaacgtgctggtggacagcaacctggtctgtaaagtgtccgacttcggcctgtccagagtgctcgaggacgaccccgacgccgcttacaccacaacaggcggcaagatccccatcagatggaccgcccctgaggctatcgccttcagaacctttagcagcgccagcgatgtgtggtccttcggagtggtcatgtgggaggttctggcctacggcgaacgaccttactggaatatgaccaacagagacgtgatcagctctgtggaagaaggctatagactgcctgctcctatgggctgtcctcacgccctccatcagctgatgctggactgctggcacaaggacagagcccagcggcctagattcagccagatcgtgagtgtgttggatgcactgatccggagccctgagagcctgagagctacagccacagtgtcccggtgccctccacctgccttcgtcagaagctgcttcgatctgagaggaggcagcggcggcggcggcggactgacagtgggcgactggctggattctatcagaatgggccgctacagagatcacttcgccgccggaggctacagcagcctggggatggtgctgaggatgaacgcccaagatgtgcgggccctgggcatcaccctgatgggacaccagaagaaaattctgggctctatccagaccatgcgggcccagctgaccagcacccagggccctagaagacatctg

SEQ ID NO: 60 - protein, 637 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLIAGSAAAGVVFVVSLVAISIVCSRKRAYSKEAVYSDKLQHYSTGRGSPGMKIYIDPFTYEDPNEAVREFAKEIDVSFVKIEEVIGAGEFGEVYKGRLKLPGKREIYVAIKTLKAGYSEKQRRDFLSEASIMGQFDHPNIIRLEGVVTKSRPVMIITEFMENGALDSFLRQNDGQFTVIQLVGMLRGIAAGMKYLAEMNYVHRDLAARNILVNSNLVCKVSDFGLSRYLQDDTSDPTYTSSLGGKIPVRWTAPEAIAYRKFTSASDVWSYGIVMWEVMSFGERPYWDMSNQDVINAIEQDYRLPPPMDCPAALHQLMLDCWQKDRNSRPRFAEIVNTLDKMIRNPASLKTVATITAVPSQPLLDRSIPDFTAFTTVDDWLSAIKMVQYRDSFLTAGFTSLQLVTQMTSEDLLRIGITLAGHQKKILNSIHSMRVQISQSPTAMA

SEQ ID NO: 61 - DNA, 1911 bp, synthetic construct
  source 1..1911: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagactgatcgccggaagcgccgccgctggagtggtctttgtggtgtccctggtggccatcagcatcgtgtgcagcagaaagcgggcctatagcaaggaggccgtgtacagcgacaagcttcaacactacagcaccggcagaggctcccctggcatgaagatctacatcgaccctttcacctacgaggatcctaacgaggccgtgcgggagttcgccaaggaaatcgacgtgtccttcgtgaagatcgaagaagtgattggcgcaggcgagttcggcgaggtttataagggcagactgaaactgccaggcaaaagagagatctacgtggccatcaagaccctgaaggccggctactccgaaaagcagcggagagacttcctgagtgaggcctctatcatgggccaatttgaccaccccaacatcattagactggaaggcgtggtgaccaaaagcagacccgtgatgatcatcaccgagttcatggaaaacggcgccctggatagcttcctgcgacagaacgacggccagtttacagtgatccagctggtgggcatgctgcggggcatcgctgccggaatgaagtacctggctgaaatgaactacgtgcacagagatctggccgccagaaacatcctggtgaatagcaatctggtgtgcaaggtgagcgattttggactgagccggtacctgcaggatgacacctctgatcccacctacaccagcagcctgggcggcaagatccccgtcagatggaccgcccctgaggctattgcctacaggaagttcaccagcgcctctgacgtctggtcctacggcatcgtgatgtgggaggtgatgagcttcggagagcggccttactgggacatgagcaaccaggacgtgatcaacgccatcgagcaggactacagactccctccacctatggactgccctgccgctctgcaccagctgatgctggactgttggcagaaggacagaaacagtcgcccccggttcgccgagatcgtgaacaccctggacaagatgatcagaaatcctgcttctctgaaaaccgttgctacaatcaccgccgtgcccagccagcctctgctggatagaagcatccctgatttcaccgccttcacaacagtggacgactggctgtctgccatcaagatggtgcagtaccgggacagcttcctgaccgccggcttcacaagcctgcaactggtcacacagatgacctccgaagatctgctgagaatcgggatcacactggccggccaccagaaaaagatcctgaacagcatccattctatgagagtgcagatctctcagagccctacagctatggcc

SEQ ID NO: 62 - protein, 705 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIIGSSAAGLVFLIAVVVIAIVCNRRGFERADSEYTDKLQHYTSGHMTPGMKIYIDPFTYEDPNEAVREFAKEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPNSLKAMAPLSSGINLPLLDRTIPDYTSFNTVDEWLEAIKMGQYKESFANAGFTSFDVVSQMMMEDILRVGVTLAGHQKKILNSIQVMRAQMNQIQSVEGQPLARRPRATGRTKRCQPRDVTKKTCNSNDGKKKGMGKKKTDPGRGREIQGIFFKEDSHKESNDCSCGG

SEQ ID NO: 63 - DNA, 2115 bp, synthetic construct
  source 1..2115: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaatcatcggcagcagcgcagccggcctggtgttcctgatcgccgtggtggtgatcgccatcgtgtgcaacagaaggggcttcgagcgggccgacagcgagtacaccgataagctgcaacactacacgtccggccacatgaccccaggcatgaagatctacatcgaccccttcacctacgaggaccctaacgaggctgtcagagagttcgccaaggaaatcgacatcagctgcgtgaagatcgagcaagtgattggagctggcgagttcggcgaagtgtgtagcggccacctgaagctgcccggcaaaagagagatttttgtggccatcaagacactgaagtccggttatacagaaaagcagagacgggacttcctgagcgaggccagcatcatgggccagtttgatcaccccaatgtgatccacctcgagggtgttgtgactaagagcacccctgtgatgatcatcaccgagttcatggaaaacggctctctggatagcttccttagacagaacgacggccaatttacagtgatccagctggtgggcatgctgaggggcatcgccgctggaatgaagtacctggctgatatgaactacgtgcacagagatctggccgctagaaacatcctggtgaacagcaatctggtctgcaaggtgtccgatttcggcctgagcagattcctggaggacgacacctctgatcctacctacacatctgccctgggcggaaagatccctatccggtggaccgcccctgaggccattcagtaccggaagttcacatccgcctctgacgtgtggagctacggcatcgtgatgtgggaggtgatgagctacggagagagaccctactgggatatgacaaaccaggacgtgatcaacgccatcgagcaggactacagactgcctccacctatggactgccccagcgccctgcaccagctgatgctggactgctggcagaaggacagaaaccaccggcccaagttcgggcagatcgtcaacaccctggacaagatgatcagaaatcctaacagcctgaaagctatggcccctctgtctagcggcatcaacctgccactgctggaccgcaccatccctgattacaccagctttaacaccgtggacgagtggctggaagccatcaagatgggccagtataaggaatctttcgccaacgccggatttaccagcttcgacgtggttagccagatgatgatggaagatatcctgcgggtgggagtgaccctggctggacatcagaaaaaaatcctgaactccattcaggtgatgagagcccagatgaaccagatccagtctgtggaaggccagcctctagccagacggcctagagccaccggccggaccaagagatgccagcccagagacgtgacaaagaagacctgtaatagcaatgatggcaagaagaaaggcatgggaaagaaaaaaacagaccctggccggggcagagagatccaaggcatcttcttcaaggaagatagccacaaggagagcaacgactgtagctgcggcggc

SEQ ID NO: 64 - protein, 632 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIVGSATAGLVFVVAVVVIAIVCLRKQRHGSDSEYTEKLQQYIAPGMKVYIDPFTYEDPNEAVREFAKEIDVSCVKIEEVIGAGEFGEVCRGRLKQPGRREVFVAIKTLKVGYTERQRRDFLSEASIMGQFDHPNIIRLEGVVTKSRPVMILTEFMENCALDSFLRLNDGQFTVIQLVGMLRGIAAGMKYLSEMNYVHRDLAARNILVNSNLVCKVSDFGLSRFLEDDPSDPTYTSSLGGKIPIRWTAPEAIAYRKFTSASDVWSYGIVMWEVMSYGERPYWDMSNQDVINAVEQDYRLPPPMDCPTALHQLMLDCWVRDRNLRPKFSQIVNTLDKLIRNAASLKVIASAQSGMSQPLLDRTVPDYTTFTTVGDWLDAIKMGRYKESFVSAGFASFDLVAQMTAEDLLRIGVTLAGHQKKILSSIQDMRLQMNQTLPVQV

SEQ ID NO: 65 - DNA, 1896 bp, synthetic construct
  source 1..1896: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaattgtggggagcgctacagctggactggtgttcgtggtggccgtggtggtgatcgctatcgtgtgcctgcggaagcagcggcacggcagcgatagcgagtacaccgagaagctgcagcagtacatcgcccctggaatgaaagtgtacatcgaccccttcacctacgaggatcctaacgaggccgttagagagttcgccaaggaaatcgacgtgtcctgcgtgaagatcgaggaagtgatcggcgccggcgaatttggcgaggtttgtagaggcagactgaaacagcctggcaggagggaggtgttcgtggccatcaagactctgaaggtcggctacaccgaaagacagagacgggacttcctgagcgaggcctccatcatgggccagtttgaccaccctaatatcatcagactggaaggcgtggtcaccaagagccggcccgtgatgatcctgaccgagttcatggaaaactgcgccctggacagctttctgcggctcaacgacggccagttcacagtgatccagctcgtgggaatgctgcgcggaatcgccgccggcatgaaatacctgagcgagatgaactacgtgcatagagatctggctgccagaaacatcctggttaacagcaacctggtgtgtaaagtgtccgatttcggcctgagcagattcctggaagatgacccttctgatcctacctacaccagcagcctcggcggcaagatccccatccggtggaccgcccctgaggccatcgcttatagaaagttcacaagcgccagcgacgtgtggtcctacggcatcgtgatgtgggaggtgatgtcttatggcgagagaccctactgggacatgagcaatcaagatgtgatcaacgccgtcgagcaggactacagacttccacctccaatggactgccccaccgccctgcaccagctgatgctggattgctgggtgagagacagaaatctgcggcctaagttcagccaaatcgtgaacaccctggataagctgattagaaacgccgcatctctgaaggtgatcgcctctgctcaaagcggcatgagccagcccctgctggaccggaccgtgcctgactacacaacattcaccacagtgggcgactggctggacgccatcaagatgggcagatacaaggaaagcttcgtgtctgccggatttgcctccttcgacctggtcgcccagatgacagccgaggacctgctgagaatcggagtgaccctggccggccaccagaaaaagatcctgtctagcatccaggacatgagactgcagatgaaccagaccctgcctgtgcaggtg

SEQ ID NO: 66 - protein, 620 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLVIGSILGALAFLLLAAITVLAVVFQRKRRGTGYTEQLQQYSSPGLGVKYYIDPSTYEDPCQAIRELAREVDPAYIKIEEVIGTGSFGEVRQGRLQPRGRREQTVAIQALWAGGAESLQMTFLGRAAVLGQFQHPNILRLEGVVTKSRPLMVLTEFMELGPLDSFLRQREGQFSSLQLVAMQRGVAAAMQYLSSFAFVHRSLSAHSVLVNSHLVCKVARLGHSPQGPSCLLRWAAPEVIAHGKHTTSSDVWSFGILMWEVMSYGERPYWDMSEQEVLNAIEQEFRLPPPPGCPPGLHLLMLDTWQKDRARRPHFDQLVAAFDKMIRKPDTLQAGGDPGERPSQALLTPVALDFPCLDSPQAWLSAIGLECYQDNFSKFGLCTFSDVAQLSLEDLPALGITLAGHQKKLLHHIQLLQQHLRQQGSVEV

SEQ ID NO: 67 - DNA, 1860 bp, synthetic construct
  source 1..1860: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagactggtgatcggctctatcctgggcgccctggccttcctgctcctggcggccatcaccgtgctggctgttgtgttccaacgaaaaagacggggcaccggctacaccgagcagctccagcagtacagcagccctggcctgggcgttaagtactacatcgaccccagcacctacgaggacccctgccaggccatccgggagctggcccgggaagtggaccctgcctatatcaagatcgaggaagtgatcggcacaggcagcttcggcgaggtccggcagggcagactgcagcctcggggccgcagagagcagaccgtggctatccaggccctgtgggccggcggagccgagtctctgcagatgaccttcctgggcagagccgccgtgctgggccagtttcagcatcctaacatcctgcggctggaaggagtggtgaccaagtccagacccctgatggtgctgaccgagttcatggagctgggacctctggacagctttctgagacagagagaaggccaattctccagtctgcagctggtggccatgcagcggggcgtggctgccgctatgcagtacctgagcagcttcgccttcgtgcacagaagcctgtctgcccacagcgtcctggtgaacagccacctggtgtgcaaggtggccaggctgggacattctccacagggccctagctgcctgctgagatgggctgcccctgaagtgatcgcccacggcaagcacaccacaagctctgatgtgtggtccttcggcatcctgatgtgggaagtgatgagctacggcgaaagaccatactgggacatgagcgagcaggaggtcctgaacgccatcgagcaggagtttagactgccccccccccccggttgtcctccaggcctgcacctgctgatgctggacacatggcagaaggacagagccagaagacctcacttcgaccagctggtggccgcttttgataagatgatcagaaaacctgatacactgcaggccggaggagatcctggagagcggcctagccaggccctgctcacccctgtggccctggacttcccttgtctggatagcccccaagcctggctgtccgccattggtctcgagtgctaccaggacaatttcagcaagttcggcctgtgcaccttcagcgacgtggcccagctgagcctggaagatctgcctgctctgggcatcacactggctggccaccagaaaaagctgctgcaccacattcagctgctgcagcagcaccttagacaacaaggctctgtggaagtg

SEQ ID NO: 68 - protein, 689 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRILIGCLVAIILLLLLIIALMLWRLHWRRLLSKAERRVLEEELTVHLSVPGDTILINNRPGPREPPPYQEPRPRGNPPHSAPCVPNGSALLLSNPAYRLLLATYARPPRGPGPPTPAWAKPTNTQAYSGDYMEPEKPGAPLLPPPPQNSVPHYAEADIVTLQGVTGGNTYAVPALPPGAVGDGPPRVDFPRSRLRFKEKLGEGQFGEVHLCEVDSPQDLVSLDFPLNVRKGHPLLVAVKILRPDATKNARNDFLKEVKIMSRLKDPNIIRLLGVCVQDDPLCMITDYMENGDLNQFLSAHQLEDKAAEGAPGDGQAAQGPTISYPMLLHVAAQIASGMRYLATLNFVHRDLATRNCLVGENFTIKIADFGMSRNLYAGDYYRVQGRAVLPIRWMAWECILMGKFTTASDVWAFGVTLWEVLMLCRAQPFGQLTDEQVIENAGEFFRDQGRQVYLSRPPACPQGLYELMLRCWSRESEQRPPFSQLHRFLAEDALNTV

SEQ ID NO: 69 - DNA, 2067 bp, synthetic construct
  source 1..2067: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaattctgatcggctgtctggtggccatcatcctgctgctgctgctgatcatcgccctgatgctgtggcggcttcactggagaagactgctgtctaaggccgagcggcgggtgctggaagaggaactgacagtgcacctcagcgtgcctggcgacaccatcctgattaacaacagaccaggccctagagagccacccccctaccaggagcctagaccccggggcaatcctcctcacagcgccccttgcgtgccaaatggcagcgccctgctgctgagcaaccccgcttatagactgctgctggccacatacgccagacctcctagaggccccggtcctcctacccctgcctgggccaagcctacaaacacccaggcctacagcggcgactacatggaacctgagaaacctggcgcccctctgctgcctcctccaccacagaacagcgtgccccactacgcagaagccgacatcgtgaccttgcagggcgtgaccggcggcaacacctacgctgttcctgccctcccccctggcgccgtgggcgatggaccacctcgggtggattttcctcggagccggctgaggtttaaggaaaagctgggcgagggacagtttggcgaagtgcacctgtgcgaggtggacagccctcaggacctggtgtccctggacttccccctgaacgtgcggaagggccaccccctgctggtggcagtgaaaatcctgagacccgacgccaccaagaacgccagaaacgacttcctgaaagaggtgaagatcatgagcagactgaaggaccccaacatcatcagactgttgggcgtgtgcgtgcaggacgaccctctgtgtatgatcaccgattacatggaaaatggcgacctgaaccagttcctgtccgcccaccagctcgaggacaaggccgctgagggcgctcctggagatggccaagctgctcaaggacctacaatcagctaccccatgctgctccacgtggccgcccagatcgcctctggcatgagatacctggccacactgaatttcgtgcatagagatctggccaccagaaactgcctggttggcgaaaacttcaccatcaagatcgccgattttggaatgtctcgcaatctgtatgccggcgattactacagagtgcaggggagagccgtgctgcccatccggtggatggcctgggagtgcatcctgatgggcaagttcacaacagccagcgacgtgtgggctttcggagttaccctgtgggaagtcctgatgctgtgcagagcccaacctttcggccagctgaccgacgagcaggtgatcgagaacgctggcgagttcttcagagaccagggccgacaggtgtacctgtctagaccccctgcttgtcctcagggactgtacgagctgatgctgcggtgctggtcccgggagagcgagcagagacctccattcagccagctgcacagattcctggctgaagatgccctgaacaccgtc

SEQ ID NO: 70 - protein, 681 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRTSFILTIIVGIFLVVTIPLTFVWHRRLKNQKSAKEGVTVLINEDKELAELRGLAAGVGLANACYAIHTLPTQEEIENLPAFPREKLTLRLLLGSGAFGEVYEGTAVDILGVGSGEIKVAVKTLKKGSTDQEKIEFLKEAHLMSKFNHPNILKQLGVCLLNEPQYIILELMEGGDLLTYLRKARMATFYGPLLTLVDLVDLCVDISKGCVYLERMHFIHRDLAARNCLVSVKDYTSPRIVKIGDFGLARDIYKNDYYRKRGEGLLPVRWMAPESLMDGIFTTQSDVWSFGILIWEILTLGHQPYPAHSNLDVLNYVQTGGRLEPPRNCPDDLWNLMTQCWAQEPDQRPTFHRIQDQLQLFRNFFLNSIYKSRDEANNSGVINESFEGEDGDVICLNSDDIMPVALMETKNREGLNYMVLATECGQGEEKSEGPLGSQESESCGLRKEEKEPHADKDFCQEKQVAYCPSGKPEGLNYACLTHSGYGDGSD

SEQ ID NO: 71 - DNA, 2043 bp, synthetic construct
  source 1..2043: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaacaagctttatcctgaccatcatcgtgggcatctttctggtcgtcaccatccccctgaccttcgtgtggcaccggagactgaaaaaccagaagagcgccaaggaaggcgtcaccgtgctcatcaacgaggataaggagctggctgaactgcggggcctcgctgctggcgtgggattggctaatgcctgctacgccatccacaccctgccaactcaagaagagatcgagaacctgcctgccttccctagggaaaagctgaccctgagactgctgctcggaagcggcgcctttggcgaggtgtacgagggcaccgccgtggacatcctgggcgtgggcagcggagagattaaggtggccgtgaagaccctgaagaagggctcaacagatcaggagaagatcgagttcctgaaggaagcacatctgatgagcaagttcaaccaccctaacattctgaagcagctgggcgtgtgcctgctgaacgagcctcagtacatcatcctggaactgatggaaggcggagatcttctgacatacctgagaaaggccagaatggccacattctacggccctctgctgacactggttgacctggtggacctgtgcgtggacatcagcaagggctgtgtgtatctggaacggatgcacttcatccacagagacctggccgctagaaattgcctggtgtccgtgaaagattacaccagccctcggatcgtgaagatcggcgatttcggcctggcccgggacatctacaagaacgactactaccgcaaaagaggcgaaggactgctgccagtgcggtggatggcccctgagtctctgatggacggaatcttcaccacccagagcgacgtgtggtccttcggcattctgatctgggaaatcctgacactgggccaccagccctaccccgctcacagcaacctggacgtgctgaactacgtgcagacaggcggaagactggaaccccccagaaactgtcctgatgatctgtggaacctgatgacccagtgctgggcccaggagcctgaccaaagacctacatttcacagaatccaggaccagctgcaactgttcagaaacttcttcctgaacagcatctataagtctcgggacgaggccaacaacagcggcgtgatcaacgagagcttcgagggcgaggacggagatgtgatctgcctgaatagcgacgacatcatgcctgttgccctgatggaaaccaagaacagagagggcctgaactacatggtgctggccaccgagtgcggccagggcgaggaaaaaagcgagggccctctgggatctcaggagtccgagagctgcggcctgagaaaggaggaaaaggaaccccacgccgacaaggacttctgccaggagaaacaggtggcctactgtcctagcggtaagcccgagggcctgaattacgcctgtctgacccactctggctacggcgacggctctgac

SEQ ID NO: 72 - protein, 775 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 73 - DNA, 2325 bp, synthetic construct
  source 1..2325: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagagtcgtgaccagcgcccttgtggctgctctggtgctggccttcagcggcatcatgatcgtgtaccgcagaaagcaccaggagctacaggccatgcagatggaactgcagagccctgaatataagctgagcaagctgcggaccagcaccatcatgaccgactacaaccctaattactgcttcgccggaaagacctccagcatcagcgacctgaaggaagtgcctcggaaaaatatcaccctgatccggggcctgggccacggcgcttttggagaagtgtacgagggccaggtgtccggcatgcctaacgaccccagccccctgcaagtggccgtgaagactctgcctgaggtttgtagcgagcaggatgagctggacttcctgatggaagccctgatcatctctaagttcaaccaccagaacatcgtgcggtgcatcggcgtgtcactgcagtctctgcctagattcatcctgctggagctgatggccggcggcgacctgaagtcttttctgagagaaaccagacctagacctagccagccttccagcctggccatgctggatctgctgcacgtggcccgggacatcgcatgcggctgccagtacctggaagaaaaccactttatccaccgggatatcgccgctaggaactgcctgctcacctgtcctggccctggcagagtggccaagatcggtgatttcggcatggccagagacatctaccgggcctcttactacagaaagggcggctgtgccatgctgcctgtgaagtggatgccccccgaggccttcatggaaggtattttcacaagcaaaacagacacatggtccttcggcgtgctgctgtgggagatcttcagcctgggctacatgccttacccctctaagagcaaccaggaggtcctggagttcgtgacctccggaggcagaatggaccctccaaagaactgccctggacctgtgtacagaatcatgacccagtgctggcagcaccagcctgaggacagaccgaatttcgccatcattctggaaagaatcgagtactgtacccaggaccccgacgtgatcaacaccgccctgcctatcgagtacggacctttagtcgaggaagaggaaaaggtgcccgtgcggcccaaggaccccgagggcgttccacctctgctcgtcagccaacaagctaaaagagaggaggagagaagccctgccgctcctccacctctgcccacaaccagcagcggcaaggccgccaagaaacccaccgccgccgagattagcgtgcgggtgcccagaggccccgccgtggaaggaggacatgtgaacatggctttttctcagagcaaccctccatctgagctgcataaggtgcacggctctagaaacaagcctaccagcctgtggaaccctacatacggcagctggttcaccgagaagcctaccaagaagaacaaccccatcgctaagaaggaacctcacgatagaggcaatctgggactggagggcagctgcacagtgccccccaatgtggctacaggccgcctgccaggcgccagcctgctgctggagcccagctccctgaccgccaacatgaaagaggtgcctctgttcagactgagacacttcccttgcggcaatgtgaactacggatatcagcagcaaggcctgccactggaagccgccacagcccctggcgccggccactacgaggatacaatcctgaaaagcaagaacagcatgaaccagcctggcccc

SEQ ID NO: 74 - protein, 633 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLVLMVAVVATSTLSLLMVCGVLILVKQKKWQGLQEMRLPSPELELSKLRTSAIRTAPNPYYCQVGLGPAQSWPLPPGVTEVSPANVTLLRALGHGAFGEVYEGLVIGLPGDSSPLQVAIKTLPELCSPQDELDFLMEALIISKFRHQNIVRCVGLSLRATPRLILLELMSGGDMKSFLRHSRPHLGQPSPLVMRDLLQLAQDIAQGCHYLEENHFIHRDIAARNCLLSCAGPSRVAKIGDFGMARDIYRASYYRRGDRALLPVKWMPPEAFLEGIFTSKTDSWSFGVLLWEIFSLGYMPYPGRTNQEVLDFVVGGGRMDPPRGCPGPVYRIMTQCWQHEPELRPSFASILERLQYCTQDPDVLNSLLPMELGPTPEEEGTSGLGNRSLECLRPPQPQELSPEKLKSWGGSPLGPWLSSGLKPLKSRGLQPQNLWNPTYRS

SEQ ID NO: 75 - DNA, 1899 bp, synthetic construct
  source 1..1899: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagactggtgctgatggtcgctgttgtggccacaagcaccctgagcctgctgatggtgtgcggcgtgctgatccttgtcaaacagaaaaagtggcagggcctgcaggaaatgcggctgccgtctcctgagctggaactgagcaagctgagaacatccgccattagaaccgcccctaacccttactactgccaggtgggcctgggccccgctcagagctggcctctgcctcctggcgtgaccgaggtgtcgcccgccaacgtgaccctgctgagggccctgggccacggcgcctttggcgaggtgtacgagggccttgtgatcggcctgcctggcgatagctccccactgcaagtggcaatcaagacactgcccgagttgtgcagccctcaagacgagctggatttcctgatggaagccctgatcatcagcaagttccggcaccagaacatcgtgcggtgcgtgggactcagcctgagagctacacctagactgatcctgctggaactgatgagcggaggagatatgaagtcttttctgcgccacagccggccacatctgggccagcctagccctctggtgatgagagacctgctgcagctggcccaggatatcgcccagggatgtcactacctggaggaaaaccacttcatccaccgggacatcgccgctagaaactgcctgctgagctgcgccggcccatctagagtggccaagattggcgacttcggcatggccagagatatctaccgggcttcatattaccgtagaggcgacagagccctgctgcctgtgaagtggatgccccctgaggccttcctggaaggcatcttcacctccaagaccgacagctggtccttcggagtgctcctgtgggagatcttcagcctgggctacatgccttatcctggaagaaccaaccaggaggtcctggacttcgtggtgggcggaggcagaatggacccccccagaggctgtcctggccccgtgtaccggatcatgacccagtgctggcaacacgagcctgagctgagacccagctttgccagcatcctggaaagactgcagtactgtacccaggaccccgacgtgctgaacagcctgctgcctatggaacttggccccacccctgaagaagagggtacaagcggcctcggcaatagatctctggagtgcctgcggcctccacagcctcaggagctcagccccgagaagctgaagagctggggcggatctccattaggtccttggctgagttctggcctgaaacctctgaagtcccggggcctgcagcctcaaaatctgtggaaccccacatacagaagc

SEQ ID NO: 76 - protein, 562 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRYLYVLSGIGGLLLLLLIFIVLYKVGFFKRNLKEKMEAGRGVPNGIPAEDSEQLASGQEAGDPGCLKPLHEKDSESGGGKDRAKRSGSGATNFSLLKQAGDVEENPGPMVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIAAIVGGTVAGIVLIGILLLVIWKALIHLSDLREYRRFEKEKLKSQWNNDNPLFKSATTTVMNPKFAES

SEQ ID NO: 77 - DNA, 1686 bp, synthetic construct
  source 1..1686: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagatatctgtacgtgctgtccggcatcggcggcctgctgctgctgctcctgatcttcatcgtgctgtacaaagtggggtttttcaagcggaacctcaaggaaaagatggaagctggcagaggcgtccccaacggcatccctgccgaagatagcgagcagctggccagcggacaggaggccggagatcctggctgtcttaagcctctgcacgagaaggacagcgagagcggcggcggaaaggatagagccaagagaagcggctctggagccacaaatttcagcctgctgaaacaagccggcgacgtggaagagaaccccggccctatggtgcgcctgcctctgcagtgcgtcctgtggggctgcttactgaccgccgtgcaccctgagccacctacagcctgtagagaaaaacagtacctgatcaacagccaatgttgcagcctgtgccagcctggccagaagctggtttctgattgcaccgagttcaccgagacagagtgccttccatgcggagaaagcgagttcctggacacctggaatagagagacccactgtcaccagcacaagtactgcgaccctaacctgggcctgagggtgcagcagaaaggcacatccgagacagataccatctgcacctgtgaagagggctggcactgcacatctgaagcctgcgagtcttgtgtgctgcaccggagctgcagccctggcttcggcgtgaagcagatcgctaccggcgtgagcgacaccatctgcgagccctgtcctgtgggcttcttttctaatgtgagctctgccttcgaaaagtgccatccctggaccagctgcgagaccaaggacctggtggtccagcaagctggaaccaacaagaccgacgtggtgtgcggcccccaggacagactgagaatcgccgctatcgtgggcggcaccgtggccggcatcgtgctgattggaatcctgctgctggtgatctggaaggccctgatccacctgtccgacctgcgggaataccggagattcgagaaggaaaaactgaagtcccagtggaacaacgacaaccccctgtttaagagcgccactaccacagtgatgaaccctaagttcgccgagagc

SEQ ID NO: 78 - protein, 239 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRVSFCLVMVLLFAVDTGLYFSVKTNIRSSTRDWKDHKFKWRKDPQDK

SEQ ID NO: 79 - DNA, 717 bp, synthetic construct
  source 1..717: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagagtgtccttctgcctggtgatggtcctgctgtttgccgtggacaccggcctgtacttcagcgtgaagacaaacatcagatctagcaccagagattggaaggaccacaagttcaagtggcggaaagatcctcaggacaag

SEQ ID NO: 80 - protein, 265 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRPFFFCCFIAVAMGIRFIIMVTMGWIRGRRSRHSWEMSEFHNYNLDLKKSDFSTRWQKQRCPVVKSKCRENAS

SEQ ID NO: 81 - DNA, 795 bp, synthetic construct
  source 1..795: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagacccttctttttctgctgttttatcgccgtggctatgggcatccggttcatcatcatggtcacaatgggctggatcagaggcagacggagcagacacagctgggaaatgagcgagttccacaactacaacctggacctgaagaagtccgatttcagcacaagatggcagaaacagcggtgccctgtggtgaagagcaagtgcagagagaatgcttct

SEQ ID NO: 82 - protein, 286 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLTAEVLGIICIVLMATVLKTIVLMNKQRGTFSEVSLAQDPKRQQRKPKGNKSSISGTEQEIFQVELNLQNPSLNHQGIDKIYDCQGLLPPPEK

SEQ ID NO: 83 - DNA, 858 bp, synthetic construct
  source 1..858: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagactcacagctgaagtgctgggcatcatctgcatcgtcctgatggccacagtgctgaagaccatcgtgctgatgaacaagcagcggggcaccttcagcgaggtgtccctggcccaggaccccaaaagacagcagagaaagcctaagggcaacaaaagctctatcagcggcaccgagcaggagatcttccaagtggaactgaatctgcagaaccccagcctgaaccaccagggaattgataagatctacgactgtcagggcctgctgcctccacctgagaag

SEQ ID NO: 84 - protein, 239 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRMGLAFLVLVALVWFLVEDWLSRKRTRERASRASTWEGRRRLNTQTL

SEQ ID NO: 85 - DNA, 717 bp, synthetic construct
  source 1..717: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaatgggcctggccttcctcgtgctggtggccctggtgtggttcctggtcgaggactggctgagcagaaagcggacccgggaaagagcttctagagccagcacctgggagggcagacggagactgaacacccagacactg

SEQ ID NO: 86 - protein, 259 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRAGTVLLLRAGFYAVSFLSVAVGSTVYYQGKCLTWKGPRRQLPAVVPAPLPPPCGSSAHLLPPVPGG

SEQ ID NO: 87 - DNA, 777 bp, synthetic construct
  source 1..777: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagagctggcaccgtgctgctcctgagagccggcttctacgccgtgtccttcctgagcgtggccgtgggcagcaccgtctactaccagggcaagtgcctgacatggaagggccccagacggcagctgcccgctgtggtgcctgcccctctgcctcctccatgtggctctagcgcccacctgctgcctcctgtgcccggagga

SEQ ID NO: 88 - protein, 277 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLVPVFCGLLVAKSLVLSALLVWWGDIWWKTMMELRSLDTQKATCHLQQVTDLPWTSVSSPVEREILYHTVARTKISDDDDEHTL

SEQ ID NO: 89 - DNA, 831 bp, synthetic construct
  source 1..831: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagactcgtccccgtgttctgcggcctgctggtggccaagtctctggtgctgagcgccctgctggtttggtggggcgacatctggtggaagaccatgatggaactgcggagcctggacacccagaaagccacatgtcacctgcagcaggtgaccgacctgccttggaccagcgtgtccagccctgtggaaagagagatcctgtaccacaccgtggctagaacaaagatcagcgacgatgatgacgagcacacactg

SEQ ID NO: 90 - protein, 252 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRILLGISGTVNGILTLTLISLIMQDEERYMTLNVQSKKRSSAQTSQLTFKDYSVTLHWYK

SEQ ID NO: 91 - DNA, 756 bp, synthetic construct
  source 1..756: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaatcctgctgggcatcagcggaaccgtgaacggcatcctgacactcacactgatctccctgatcatgcaagatgaggaacggtacatgaccctgaacgtgcagagcaagaaaagatctagcgcccagaccagccagctgaccttcaaggactacagcgtgaccctgcactggtacaag

SEQ ID NO: 92 - protein, 252 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRVLIGTSVVKIPFTILLFFLLHRWCSDKKNAAVMDQEPAGNRTVNSEDSDEQDHQEVSYA

SEQ ID NO: 93 - DNA, 756 bp, synthetic construct
  source 1..756: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagagtcctgatcggcacaagcgtggtgaagatccccttcaccatcctgctgtttttcctgctgcaccggtggtgctctgataagaaaaacgccgctgtgatggaccaggagcctgccggcaatagaaccgtgaacagcgaagatagcgacgagcaggaccaccaggaagtgtcctacgcc

SEQ ID NO: 94 - protein, 252 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRFFLIMFLTIIVCGMVAALSAIMHDSNNVEKDITPSELPANPGCLHSKEHSIKATLIWRL

SEQ ID NO: 95 - DNA, 756 bp, synthetic construct
  source 1..756: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagatttttcctgatcatgttcctgaccatcatcgtgtgcggcatggtggccgccctgagcgccatcatgcacgatagcaacaacgtggaaaaggacatcacccctagcgagctgcctgctaatcccggctgtctgcactccaaagagcactctatcaaggccacactgatttggagactg

SEQ ID NO: 96 - protein, 235 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRILIGTSVVKIPFTILLFFLLHRWCSNKKKCCCNGPRACREQK

SEQ ID NO: 97 - DNA, 705 bp, synthetic construct
  source 1..705: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaatcctgatcggcacatctgtggtgaagatccccttcaccatcctgctgtttttcctgctgcaccggtggtgcagcaacaagaaaaagtgctgttgcaacggccctagagcctgcagagagcagaag

SEQ ID NO: 98 - protein, 261 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRFWVLVVVGGVLACYSLLVTVAFIIFWVRSKRSRLLHSDYMFMTPRRPGPTRKHYQPYAPPRDFAAYRS

SEQ ID NO: 99 - DNA, 783 bp, synthetic construct
  source 1..783: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagattctgggtgctggtggtggtgggaggcgtgctggcctgctactctctgctggtgaccgtggccttcatcatcttttgggtgaggagcaagaggtcccgcctgctgcacagcgactatatgttcatgacccctcggagaccaggccccacaagaaagcactaccagccctatgccccccctcgggattttgccgcctacagaagc

SEQ ID NO: 100 - protein, 262 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRIISFFLALTSTALLFLLFFLTLRFSVVKRGRKKLLYIFKQPFMRPVQTTQEEDGCSCRFPEEEEGGCEL

SEQ ID NO: 101 - DNA, 786 bp, synthetic construct
  source 1..786: mol_type = other DNA
atggcgaggctgccactgcagtgcgtgctgtggggatgtctgctgaccgcagtgcacccagagccccctacagcctgccgcgagaagcagtacctgatcaacagccagtgctgttccctgtgccagcctggccagaagctggtgagcgactgtaccgagttcaccgagacagagtgcctgccatgtggcgagtccgagtttctggacacctggaaccgggagacacactgccaccagcacaagtattgtgatcccaatctgggcctgagagtgcagcagaagggcacctctgagaccgatacaatctgcacatgtgaggagggctggcactgcacctctgaggcatgcgagagctgcgtgctgcaccggtcttgtagccccggcttcggcgtgaagcagatcgccaccggcgtgtccgacacaatctgcgagccatgtcccgtgggcttcttttctaacgtgagctccgcctttgagaagtgccacccttggacctcctgtgagacaaaggatctggtggtgcagcaggccggcaccaataagacagacgtggtgtgcggacctcaggataggctgagaatcatctccttcttcctggccctgacatctaccgccctgctgttcctgctgttttttctcacactgagattcagcgtggtgaagcggggcagaaagaaactgctgtacatcttcaagcagcctttcatgcggcctgtgcagaccacccaggaggaggacggctgcagctgcagattccccgaggaagaggaaggcggatgtgaactg

SEQ ID NO: 102 - protein, 401 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 103 - DNA, 1203 bp, synthetic construct
  source 1..1203: mol_type = other DNA
atggtgaggctgccactgcagtgcgtgctgtggggatgtctgctgaccgcagtgcacccagagccaccaacagcctgtcgcgagaagcagtacctgatcaattctcagtgctgtagcctgtgccagcccggccagaagctggtgtccgattgtaccgagttcaccgagacagagtgcctgccttgtggcgagtctgagtttctggatacctggaaccgggagacacactgccaccagcacaagtattgtgaccccaatctgggcctgagagtgcagcagaagggcacctccgagaccgacacaatctgcacatgtgaggagggatggcactgcaccagcgaggcatgcgagtcctgcgtgctgcaccgctcctgttctcccggcttcggcgtgaagcagatcgccaccggcgtgtctgatacaatctgcgagccctgtcctgtgggcttctttagcaacgtgtcctctgcctttgagaagtgccacccttggaccagctgtgagacaaaggacctggtggtgcagcaggccggcaccaataagacagatgtggtgtgcggacctcaggacaggctgagaaccatcatcggcgtgagcgtgctgtccgtgctggtggtgtctgtggtggccgtgctggtgtacaagttctactttcacctgatgctgctggccggctgcatcaagtatggcaggggcgagaacatctacgacgccttcgtgatctatagctcccaggacgaggattgggtgcgcaacgagctggtgaagaatctggaggagggcgtgccccctttccagctgtgcctgcactacagggacttcatccccggcgtggcaatcgccgccaatatcatccacgagggctttcacaagtccaggaaagtgatcgtggtggtgtcccagcacttcatccagtctcgctggtgtatctttgagtatgagatcgcccagacctggcagttcctgtctagccgggccggcatcatctttatcgtgctgcagaaggtggagaagaccctgctgagacagcaggtggagctgtacaggctgctgagccgcaacacatatctggagtgggaggactccgtgctgggccggcacatcttctggcggagactgagaaaggccctgctggatggcaagtcttggaacccagagggcaccgtgggcacaggatgtaattggcaggaggccacaagcatc

SEQ ID NO: 104 - protein, 239 aa, synthetic construct
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRLYVAFLASGIMQTILFVTLLYMFIKMSIPLGTAASRPSTQNYTEIM

SEQ ID NO: 105 - DNA, 717 bp, synthetic construct
  source 1..717: mol_type = other DNA
atggtgaggctgccactgcagtgcgtgctgtggggatgtctgctgaccgcagtgcacccagagccaccaacagcctgtcgcgagaagcagtacctgatcaattctcagtgctgtagcctgtgccagcccggccagaagctggtgtccgattgtaccgagttcaccgagacagagtgcctgccttgtggcgagtctgagtttctggatacctggaaccgggagacacactgccaccagcacaagtattgtgaccccaatctgggcctgagagtgcagcagaagggcacctccgagaccgacacaatctgcacatgtgaggagggatggcactgcaccagcgaggcatgcgagtcctgcgtgctgcaccgctcctgttctcccggcttcggcgtgaagcagatcgccaccggcgtgtctgatacaatctgcgagccctgtcctgtgggcttctttagcaacgtgtcctctgcctttgagaagtgccacccttggaccagctgtgagacaaaggacctggtggtgcagcaggccggcaccaataagacagatgtggtgtgcggacctcaggacaggctgagactgtacgtggccttcctggccagcggcatcatgcagaccatcctgttcgtgacactgctgtatatgtttatcaagatgtctatcccactgggcaccgcagccagccggccttccacccagaactacacagagatcatg